(19) United States
(12) Patent Application Publication
(10) Pub. No.: US 2010/0093536 A1
(43) Pub. Date: Apr. 15, 2010

(54) CONTROL OF GRASSY WEEDS WITH VINYLGLYCINES AND VINYLGLYCINE-PRODUCING ORGANISMS

(75) Inventors: DONALD J. ARMSTRONG, CORVALLIS, OR (US); MARK D. AZEVEDO, ALBANY, OR (US); KERRY L. MCPHAIL, CORVALLIS, OR (US); MICHAEL D. JACKSON, SALEM, OR (US); DALLICE I. MILLS, CORVALLIS, OR (US); GARY BANOWETZ, CORVALLIS, OR (US); MURALIDHARA THIMMAIAH, CORVALLIS, OR (US); ANNE B. HALGREN, CORVALLIS, OR (US)

Correspondence Address: KLARQUIST SPARKMAN, LLP, 121 SW SALMON STREET, SUITE 1600, PORTLAND, OR, 97204, US

(73) Assignees: THE STATE OF OREGON ACTING BY AND THROUGH THE STATE BOARD OF HIGHER; EDUCATION ON BEHALF OF OREGON STATE UNIVERSITY; THE GOVERNMENT OF THE UNITED STATES OF AMERICA AS REPRESENTED BY THE; SECRETARY OF THE DEPARTMENT OF AGRICULTURE

(21) Appl. No.: 12/567,590

(22) Filed: Sep. 25, 2009

Related U.S. Application Data
(60) Provisional application No. 61/101,500, filed on Sep. 30, 2008.

(51) Int. Cl. A01N 25/32; C07C 229/06; A01N 63/00; C12P 13/02

(52) U.S. Cl. 504/110; 562/450; 504/117; 435/129

(57) ABSTRACT

The present disclosure concerns specific herbicide molecules, herbicidal compositions comprising the molecules and the bacterial strains that produce certain of these molecules, and methods of using these molecules and bacterial strains to control grassy weeds and other sensitive weed species.

### CROSS REFERENCE TO RELATED APPLICATION

Benefit is claimed to U.S. Provisional Application No. 61/101,500, filed Sep. 30, 2008, which is incorporated herein by reference in its entirety.

### GOVERNMENT RIGHTS

The claimed invention was developed, at least in part, with United States government support under USDA CSREES Grant No. 00-34321-9798. The United States government has certain rights in the invention.

### FIELD

The present disclosure concerns specific herbicidal vinylglycine molecules and molecules of related structure, compositions including the molecules and the bacterial strains that produce certain of these molecules, and methods for using these molecules and bacterial strains to control grassy weeds and other sensitive weed species.

### BACKGROUND

Annual bluegrass (Poa annua), roughstalk bluegrass (Poa trivialis) and downy brome (Bromus tectorum) are serious weed problems in grass-seed production fields of the Pacific Northwest and other regions. Annual costs to remove P. annua from perennial ryegrass seed to comply with federal and state seed certification standards can exceed $30,000,000 for the Willamette Valley alone (Burr, Section 18, “Emergency Exemptions Requests for Use During the 1997-98 Field Season in Oregon Grass Seed Crops,” Oregon Seed Council, Ag. Research, Inc. 1998). Grass seed producers in Central and Eastern Oregon, as well as regions of Washington and Idaho, face similar difficulties. Increased weed seed contamination in Oregon and Washington also result from efforts to improve air quality by state-mandated reductions of open field burning.

Concurrently, controls on chemical herbicides registered for use against P. annua and other weedy grasses have become more stringent. Increased dependence on a limited number of chemicals has been accompanied by the emergence of herbicide-resistant biotypes of P. annua and B. tectorum (Gamroth, “Resistance of annual bluegrass (Poa annua L.) to diuron and ethofumesate,” M. S. Thesis, Oregon State University, Corvallis, 1997; Mueller-Warrant, “Herbicide Resistant Weeds, Prevention and Control,” 5th Grass Seed Cropping Systems for Sustainable Agriculture Review, 1998). P. annua biotypes that are resistant to herbicides also have been observed in other parts of the nation (Kelly and Coats, Proceedings, Southern Weed Science Society, 51:90, 1998a; Kelly and Coats, Proceedings, Southern Weed Science Society, 51:71, 1998b).

During the past decade, the number of registered chemical herbicides has decreased because of their adverse effects on the environment. Because of the relatively broad specificity of these compounds and their ability to persist in the environment, they may impose significant environmental risks. Recognition by the general public and environmental agencies of the need to develop environmentally friendly control strategies has placed the continued use of some chemicals in jeopardy. Moreover, annual bluegrass biotypes that are resistant to synthetic chemical herbicides have reduced the efficacy of these chemicals for control of weeds.

An alternative to herbicides is the use of deleterious rhizobacteria to suppress or reduce growth of weed populations (Boyetchko, HortScience, 32(2):201-205, 1997). For example, pathovars of Xanthomonas campestris pv. poannua are systemic pathogens of P. annua that have been used for biocontrol of annual bluegrass. However, strategies employing a live organism as the biocontrol agent have usually targeted established weed seedlings, and this approach has proven problematic in practice.

In view of these considerations, there is a need for effective alternatives for the control of grassy weeds.

### SUMMARY OF THE DISCLOSURE

Described herein is a specific bacterial bioherbicide produced, for example, by Pseudomonas fluorescens, Pseudomonas mucidolens/synxantha, and Enterobacter kobei, chemical compounds structurally related to this bioherbicide, and methods of using this bioherbicide or the structurally related chemical compounds described herein, or the bacteria that produce either the specific bioherbicide or structurally related compounds, to control germination of grassy weeds and other sensitive weed species.

One embodiment of the disclosure is bacterial strain, Pseudomonas fluorescens Biotype C WH6, wherein the bacterial strain inhibits or arrests grassy weed germination. Other embodiments of the disclosure are the bacterial strains Pseudomonas fluorescens isolates AD31, AH4, E34, WH19, AII10, BT1, E24, TR33, TR44, TR46, A3422A, ALW38, G2Y, GTR12, GTR24, GTR40, HB14, HB26, HB32, ST22, and W36; Pseudomonas mucidolens/synxantha isolates A342 and TDH40, and Enterobacter kobei isolate A3203. These bacterial strains also inhibit or arrest grassy weed germination. More generally, as taught herein any bacterium having certain characteristic biosynthetic gene sequences (for instance, the formyl-transferase described in Example 5) is also capable of producing one or more of the herbicidal compounds described herein.

Also disclosed herein is a low molecular weight natural product termed a Germination-Arrest Factor (GAF), identified as 2-amino-4-formylaminooxy-but-3-enoic acid (also named 4-formylaminooxyvinylglycine). GAF is produced by any bacterium having certain characteristic biosynthetic gene sequences (characterized by for instance, the formyl-transferase described in Example 5), including for instance Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, and Enterobacter kobei A3203. GAF inhibits or arrests grassy weed germination.

Also disclosed herein is a method of inhibiting or arresting weed germination. The method includes applying a bacterium that produces GAF, such as Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, and Enterobacter kobei A3203, or mixtures thereof, to a growth medium in which it would be desirable to inhibit or arrest weed germination, thereby inhibiting or arresting weed germination.

Still further embodiments of the disclosure are methods of inhibiting or arresting weed germination that include applying the isolated natural product GAF, or chemical compounds that are structurally related to GAF, to a growth medium in which it would be desirable to inhibit or arrest weed germination, thereby inhibiting or arresting weed germination.

A further embodiment of the disclosure is a method of using the isolated natural product GAF, or chemical compounds that are structurally related to GAF, as a seed-cleaning adjuvant in seed-cleaning processes as a supplement or alternative to physical removal of target weed seeds.

Further embodiments are compositions for inhibiting or arresting the germination of weeds. The compositions of this disclosure include the isolated natural product GAF, or chemical compounds that are structurally related to GAF, and a timed-release coating that is coating GAF or compounds that are structurally related to GAF.

Still another embodiment of the disclosure is a method of inhibiting or arresting weed germination in a grass patch. The method includes broadcasting the isolated natural product GAF, or chemical compounds that are structurally related to GAF, at least once a year across a grass field in which inhibiting or arresting weed germination is desirable, thereby inhibiting or arresting weed germination.

Yet another embodiment of the disclosure is a method of producing GAF. The method includes culturing any bacterium having certain characteristic biosynthetic gene sequences (characterized by for instance, the formyl-transferase described in Example 5), such as for instance any of Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, and Enterobacter kobei A3203, in a suitable culture medium, collecting the culture medium, and separating components in the culture medium to produce purified GAF.

Also disclosed is a kit for inhibiting or arresting weed growth. The kit includes isolated GAF (or chemical compounds that are structurally related to GAF or formulations of bacteria that produce GAF), a container, and, optionally, instructions for using the kit.

Still other embodiments are bacterial strains having the GAF-producing characteristics of P. fluorescens isolates WH6 (NRRL# B-30485), AD31 (NRRL# B-30483), AH4 (NRRL#B-30482), E34 (NRRL# B-30481), WH19 (NRRL# B-30484), AH10 (NRRL# B-50232), BT1 (NRRL#B-50230), E24 (NRRL# B-50229), TR33 (NRRL# B-50220), TR44 (NRRL# B-50219), TR46 (NRRL# B-50218), A3422A (NRRL# B-50234), ALW38 (NRRL# B-50231), G2Y (NRRL# B-50228), GTR12 (NRRL# B-50227), GTR24 (NRRL# B-50226), GTR40 (NRRL# B-50225), HB14 (NRRL# B-50224), HB26 (NRRL# B-50223), HB32 (NRRL# B-50222), ST22 (NRRL# B-50221) and W36 (NRRL# B-50217); Pseudomonas mucidolens/synxantha A342 (NRRL# B-50236) and TDH40 (NRRL# B-50235); or Enterobacter kobei A3203 (NRRL# B-50233), when these bacterial strains are used to produce GAF or used directly as biocontrol agents to control weed germination.

Other embodiments of the disclosure are isolated bacteria that produce vinylglycine molecules other than GAF, when these bacterial strains are used as biocontrol agents to control weed germination.

Still another embodiment of the disclosure is a method of inhibiting weed germination by the use of formulations that contain compounds that are structurally unrelated to GAF but share with GAF and other vinylglycines the ability to inhibit pyridoxal-phosphate dependent enzyme reactions.

### BRIEF DESCRIPTION OF THE FIGURES

FIG. 1 includes a comparison of the ES+ Mass Spectra of TLC-Purified GAF (panel A) and NaH₂PO₄ (panel B).

FIG. 2 is a comparison of elution profiles of iron and copper binding activity as measured with the corresponding ChromeAzurol S reagent. A sample (25 mL) of TLC-purified GAF (4× concentration) was evaporated to dryness in vacuo (≦45° C.), and the solids were dissolved in 2.5 mL deionized water (to give a 40× solution). A 1.4 mL aliquot of this solution was applied to the Sephadex® G10 column, which was eluted with deionized water. Fractions were assayed for iron and copper binding using the corresponding Chrome Azurol S reagent and then pooled (A-D) for TLC analysis.

FIG. 3 shows the results of TLC analysis of the pooled fractions A-D from the Sephadex® G10 Column fractionation shown in FIG. 2. For TLC analysis, each pooled fraction was taken to dryness in vacuo (≦45° C.) and redissolved in 0.6 mL of 76% ethanol. Aliquots (200 μL each) of these solutions were applied to analytical micro-crystalline cellulose TLC plates and chromatographed as described in the text. The developed plates were stained with ninhydrin. The ninhydrin-positive band appearing in C, and more weakly in B, corresponds to GAF.

FIG. 4 illustrates the purification of GAF for mass spectrometric analysis samples using the first of three consecutive Sephadex® G15 columns. TLC-purified GAF from WH6 culture filtrate (46 mL, 4× concentration relative to culture filtrate) was evaporated in vacuo (≦45° C.) and redissolved in 3.06 mL deionized water to give a 60× solution. A 2.4 mL aliquot of this solution was applied to a Sephadex® G15 column and eluted with deionized water. Fractions were collected and assayed for metal binding.

FIG. 5 illustrates the second of three consecutive purifications of GAF via chromatography. In this purification the collected fractions from Sephadex® G15 Column 1 were evaporated to dryness in vacuo (≦45° C.) and redissolved in 2.0 mL deionized water. This sample was rechromatogaphed on a Sephadex® G15 column as indicated.

FIG. 6 illustrates the third of three consecutive purifications of GAF via chromatography. In this purification the collected fractions from Sephadex® G15 Column 2 were evaporated to dryness in vacuo (≦45° C.) and redissolved in 2.0 mL deionized water. This sample was rechromatogaphed on a Sephadex® G15 column as indicated. TLC analysis of the isolated eluate revealed a single ninhydrin-positive band as shown in FIG. 7.

FIG. 7 shows the results of TLC analysis of the purified GAF sample from the Sephadex® G15 fractionation shown in FIG. 6. A 50 μL aliquot of the GAF sample (1.2 mL in 60% ethanol) prepared for mass spectrometric analysis by pooling and concentrating the indicated fractions from Sephadex® G15 Column 3 shown in FIG. 6 was analyzed by analytical TLC chromatography and ninhydrin staining according to the protocol described in the text.

FIG. 8 illustrates a representative chromatographic purification of a GAF sample for Nuclear Magnetic Resonance (NMR) analysis, showing the first of three successive Sephadex® G15 columns. TLC-purified GAF from WH6 culture filtrate (150 mL, 4× concentration relative to culture filtrate) was evaporated in vacuo (≦45° C.) and redissolved in 6.0 mL deionized water to give a 100× solution. The 6.0 mL sample was applied to a Sephadex® G15 column and eluted with deionized water. Fractions were collected as indicated and were assayed for metal binding.

FIG. 9 illustrates the second of three consecutive purifications of GAF via chromatography on Sephadex® G15 to prepare a sample for NMR spectroscopic analysis. The indicated pooled fractions from Sephadex® G15 Column 1 (FIG. 6) were evaporated to dryness in vacuo (≦45° C.) and redissolved in 5.0 mL deionized water. This sample was rechromatogaphed on a Sephadex® G15 column.

FIG. 10 illustrates the third of three consecutive purifications of GAF via chromatography to prepare a sample for NMR spectroscopic analysis. The indicated pooled fractions from Sephadex® G15 Column 2 (FIG. 7) were evaporated to dryness in vacuo (≦45° C.) and redissolved in 5.0 mL deionized water. This sample was rechromatogaphed on a Sephadex® G15 column.

FIG. 11 shows the results of TLC analysis of GAF samples purified for NMR analysis. TLC analyses of each of the two NMR samples generated by the Sephadex® G15 fractionations as shown in FIGS. 8-10. Aliquots (200 μL) were taken from each of the two 10 mL 76% ethanol sample solutions and chromatographed on separate TLC plates in the standard analytical TLC system used for GAF analysis as described in the text. The developed plates were stained with ninhydrin. The ninhydrin-positive bands on the plates correspond to GAF.

FIG. 12 is a ¹³C NMR spectrum of GAF in D₂O at 75 MHz.

FIG. 13 is a ¹H NMR spectrum of GAF in D₂O at 300 MHz.

FIG. 14 is a ¹H—¹H gCOSY spectrum of GAF in D₂O at 300 MHz.

FIG. 15 is a multiplicity-edited HSQC NMR spectrum of GAF in D₂O at 300 MHz.

FIG. 16 is an HMBC NMR spectrum of GAF in D₂O (optimized for J_{CH}=8 Hz: d6=65 ms) at 300 MHz.

FIG. 17 is an HMBC NMR spectrum of GAF in D₂O (optimized for J_{CH}≈2 Hz: d6=125 ms) at 300 MHz.

FIG. 18 is an HMBC NMR spectrum of GAF in D₂O (optimized for J_{CH}≈12 Hz: d6=45 ms) at 300 MHz.

FIG. 19 includes predicted ¹³C NMR spectra for two structural alternatives shown generated using ChemDraw Ultra 9.0.

FIG. 20 illustrates the results of double pulsed field gradient spin echo selective 1D NOESY NMR of GAF in D₂O: spectrum A demonstrates that selective excitation of H-4 produces enhancement of H-2; spectrum B demonstrates that selective excitation of H-3 produces minimal enhancement of H-2.

FIG. 21 is an ES+ mass spectrum of a purified GAF sample.

FIG. 22 is a delayed ES+ mass spectrum of purified GAF sample.

FIG. 23 is an ES+ mass spectrum of a control sample.

FIG. 24 is a tandem ES+ mass (MS²) fragmentation spectrum of m/z 321 from the mass spectrum of purified GAF.

FIG. 25 is a tandem ES+ mass (MS²) fragmentation spectrum of m/z 343 from the mass spectrum of purified GAF.

FIG. 26 is a tandem ES+ mass (MS²) fragmentation spectrum of m/z 547 from the mass spectrum of purified GAF.

FIG. 27 is a tandem ES+ mass (MS²) fragmentation spectrum of m/z 183 from the mass spectrum of purified GAF including assigned peaks.

FIG. 28 is an ES+ MS³ fragmentation spectrum of the m/z 165.7 fragment of the m/z 183 fragmentation illustrated in FIG. 24 from the mass spectrum of purified GAF.

FIG. 29 is an ES+ MS³ fragmentation spectrum of the m/z 138.6 fragment of the m/z 183 fragmentation illustrated in FIG. 24 from the mass spectrum of purified GAF.

FIG. 30 is an ES+ MS³ fragmentation spectrum of the m/z 122.9 fragment of the m/z 183 fragmentation illustrated in FIG. 24 from the mass spectrum of purified GAF.

FIG. 31 is an ES+ MS³ fragmentation spectrum of the m/z 94.7 fragment of the m/z 183 fragmentation illustrated in FIG. 24 from the mass spectrum of purified GAF.

FIG. 32 includes digital images illustrating the effect of AVG on germination of seeds of Poa annua in a soil-based system.

FIG. 33 includes digital images illustrating the effect of AOA on germination of seeds of Poa annua in a soil-based system.

FIG. 34 includes digital images illustrating the effect of AVG on germination of seeds of Poa annua in a soil-based system with established perennial ryegrass (Lolium perenne) seedlings.

FIG. 35 includes digital images illustrating the effect of AOA on germination of seeds of Poa annua in a soil-based system with established perennial ryegrass (Lolium perenne) seedlings.

FIG. 36 is a graph illustrating the interaction of WH6 Culture Filtrate with Alanine in the Standard GAF Bioassay. Pseudomonas fluorescens WH6 culture filtrate was mixed with alanine and tested in the Poa bioassay at the concentrations indicated. A Germination Score of 1.0 indicates high GAF activity (germination completely arrested immediately after emergence of the coleorhiza and plumule). A Germination Score of 4.0 indicates no GAF activity (germination and seedling development equivalent to that of controls). See the text for details of the bioassay procedure and the scoring system.

FIG. 37 is a graphic alignment of the 417-bp 16S-rDNA sequence used for taxonomic classification with the complete 16S-rRNA gene of P. fluorescens PfO-1.

FIG. 38 shows a Thin-Layer Chromatography (TLC) analysis of culture filtrates from Pseudomonas isolates of Group I. Culture filtrates were prepared, extracted, and chromatographed on cellulose TLC plates as described in Example 1. The developed chromatograms were sprayed with ninhydrin reagent as described in the same source. The position of the Germination-Arrest Factor band is indicated for WH6.

FIG. 39 shows a Thin-Layer Chromatography (TLC) analysis of culture filtrates from Pseudomonas Isolates of Group II as described in Example 5. Culture filtrates were prepared, extracted, and chromatographed on cellulose TLC plates as described in Example 1. The developed chromatograms were sprayed with ninhydrin reagent as described in the same source. The chromatogram prepared from WH6 culture filtrate is included as a reference, and is shown alongside chromatograms prepared from culture filtrate of isolates AH10, BT1, and E24 (FIG. 39A). Also shown are chromatograms prepared from culture filtrate of isolates TDH5, TR33, TR44, and TR46 (FIG. 39B)

FIG. 40 shows a Thin-Layer Chromatography (TLC) analysis of culture filtrates from Pseudomonas strains obtained from other laboratories. Culture filtrates were prepared, extracted, and chromatographed on cellulose TLC plates as described in Example 1. The developed chromatograms were sprayed with ninhydrin reagent as described in the same source. The chromatogram prepared from WH6 culture filtrate is included as a reference.

FIG. 41 shows the distribution of the GAF-specific formyl-transferase gene in various Pseudomonas strains and isolates. The DNA primer sequences set forth as SEQ ID NOs: 3 and 4 were used to test the P. fluorescens isolates and strains listed in Tables 1 and 2 for the presence of a 316 nucleotide sequence homologous to the corresponding region of the formyl-transferase gene from P. fluorescens WH6. DNA isolation and PCR procedures are described in Example 1.

FIG. 42 shows the analysis of DNA from Pseudomonas fluorescens strain-D7 for the presence of the GAF-specific formyl-transferase gene. The DNA primer sequences set forth as SEQ ID NOs: 3 and 4 were used to test DNA from P. fluorescens Strain D7 for the presence of a 316 nucleotide sequence homologous to the corresponding region of the formyl-transferase gene from P. fluorescens WH6. P. fluorescens WH6 was included as a positive control. DNA isolation and PCR procedures are described in Example 1.

FIG. 43 shows the ¹H NMR spectrum (in D₂O, at 300 MHz) for a crude 90% ethanol extract of the culture filtrate from Pseudomonas fluorescens Isolate WH6. Arrows indicate characteristic FVG peaks.

FIG. 44 shows the ¹H NMR spectrum (in D₂O, at 300 MHz) for a crude 90% ethanol extract of the culture filtrate from Pseudomonas fluorescens Isolate GTR24. Arrows indicate characteristic FVG peaks.

FIG. 45 shows the ¹H NMR spectrum (in D₂O, at 300 MHz) for a crude 90% ethanol extract of the culture filtrate from Enterobacter kobei Isolate A3203. Arrows indicate characteristic FVG peaks.

FIG. 46 shows the ¹H NMR spectrum (in D₂O, at 300 MHz) for a crude 90% ethanol extract of the culture filtrate from Pseudomonas fluorescens Isolate TDH5.

FIG. 47 shows the ¹H NMR spectrum (in D₂O, at 300 MHz) for a crude 90% ethanol extract of the culture filtrate from Pseudomonas fluorescens Strain PfO-1.

### SEQUENCE LISTING

The disclosed nucleic acid sequences referenced herein are shown using standard letter abbreviations for nucleotide bases as defined in 37 C.F.R. 1.822. In the accompanying sequence listing:

SEQ ID NO: 1 is a segment of the 16S- rRNA gene of Pseudomonas fluorescens isolate WH6.

SEQ ID NO: 2 is the nucleotide sequence of the putative formyl-transferase from Pseudomonas fluorescens WH6.

SEQ ID NOs: 3 and 4 are oligonucleotides used to amplify a subsequence of the putative formyl transferase from Pseudomonas fluorescens WH6.

### DETAILED DESCRIPTION

### I. Abbreviations

ExPASy: Expert Protein Analysis System

FAME: Fatty Acid Methyl Ester Analysis

FPM: Fluorescent Pseudomonas Medium

GAF: Germination-Arrest Factor

h: hour

OD: Optical Density

ORF: Open Reading Frame

mL: milliliter

μM: micromolar

mm: millimeter

mM: millimolar

m²: square meters

NMR: nuclear magnetic resonance

PCR: polymerase chain reaction

PMS: Pseudomonas Minimal Salt Medium

QSAR: Quantitative Structure Activity Relationships

s: second

SCFU: Single Colony Forming Units

SIB: Swiss Institute of Bioinformatics

TLC: thin-layer chromatography

UV: Ultraviolet

v/v: volume-to-volume

w/v: weight-to-volume

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Adjuvant: A chemical added to an herbicide formulation or tank mix to improve mixing and application or enhance performance. Most herbicide formulations contain at least a small percentage of adjuvants. Wetting agents and spreaders are the adjuvants most frequently added. Common adjuvants include, but are not limited to, wetting agents, such as anionic, cationic, nonionic, and amphoteric surfactants, stabilizers, preservatives, antioxidants, extenders, solvents, emulsifiers, invert emulsifiers, spreaders, stickers, penetrants, foaming agents, anti-foaming agents, thickeners, safeners, compatibility agents, crop oil concentrates, viscosity regulators, binders, tackers, drift control agents, or other chemical agents, such as fertilizers, antibiotics, fungicides, nematicides, or pesticides.

Annual Bluegrass (Poa annua): A low-growing, cool season grass that dies early in the summer when the top soil dries out. Annual bluegrass can be distinguished easily from other grasses by its typical leaf tip, which is shaped like the bow of a boat. In addition, the leaf blade is often crinkled at midsection. The mature plant grows as dense, low-spreading tufts, 8 to 30 cm tall, and often roots at the lower nodes.

Annual bluegrass begins to emerge in late summer and early fall when night temperatures are in the range of 15° C. (60° F.) and moisture is abundant. Annual bluegrass seeds continue to germinate through the fall, winter and spring, a characteristic that makes chemical control more difficult. Germination and growth of annual bluegrass are favored by moist soil conditions and cool temperatures. Thus, it has a strong competitive advantage over warm season grasses from fall through spring. On closely mowed and irrigated turf, annual bluegrass will dominate a stand of Bermuda grass by late spring if herbicides are not used. Populations of annual bluegrass are greatly reduced by taller mowing heights and limited use of water.

Annual bluegrass initiates seedheads in late fall and winter, but seedhead development is greatest in the spring and early summer. After seedhead appearance, the turf develops a yellowish-white color and an uneven appearance.

Annual bluegrass is best controlled with pre- and post-emergence herbicides in warm season turf grasses. Preemerge products generally are applied prior to the emergence of annual bluegrass in the fall. The date of emergence varies between locations and years, but generally begins when night temperatures are in the range of 60-70° C. and daytime temperatures are below 85° F. Where winter grasses are to be overseeded on Bermuda grass turf, applications of preemergence products generally are made 60 to 90 days before seeding. Annual bluegrass also can be controlled using the compounds and methods described herein.

Antibiotic: A substance, for example penicillin or streptomycin, often produced by or derived from certain fungi, bacteria, and other organisms, that can destroy or inhibit the growth of other microorganisms.

Anti-foaming agent: An adjuvant that reduces foaming of spray mixtures that require vigorous agitation.

Antioxidant: An adjuvant, such as vitamin E, vitamin C, or beta carotene that protects a compound, for example an herbicide, from the damaging effects of oxidation.

Bioherbicide: A preparation of living inoculum of a plant pathogen or other microorganism, or a compound produced by such a pathogen or other microorganism, that is formulated and applied in a manner analogous to that of a chemical herbicide in an effort to suppress the growth of weed species. The use of bioherbicides is based on the fundamental epidemiological principles of plant pathology.

Pathogen factors such as low inoculum levels, weakly virulent pathogens, and poor spore dispersal mechanisms; environmental factors such as unfavorable moisture and/or temperature conditions; and plant factors such as low susceptibility of the host, and widely dispersed host populations often limit disease. The bioherbicide approach is an attempt to bypass many of these restraints on disease development by periodically dispersing an abundant supply of virulent inoculum, or a compound produced by a plant pathogen or other microorganism, uniformly onto a susceptible weed population. Generally, the application is timed to take advantage of favorable environmental conditions and/or the most susceptible stage of plant growth. Similarly, the bioherbicide generally is formulated to avoid unfavorable environmental conditions and to facilitate application.

Buffer: An ionic compound that resists changes in its pH.

Compatibility Agent: An adjuvant that aids in combining herbicides effectively.

Crop Oil Concentrate: A phytobland petroleum or vegetable oil that increases absorption of an herbicide through the cuticle of leaves.

Dicot: Any flowering plant with two embryonic seed leaves (cotyledons). Cotyledons usually appear at germination.

Downy Brome (Bromus tectorum): An erect winter- or spring-annual grass. The seedlings are bright green with conspicuously hairy leaves, hence the common name, downy brome. At maturity, the foliage and seedheads often become purplish before drying completely and becoming brown or tan. The species grows quickly in the spring and often matures and sets seeds before most other species. It typically grows 50-60 cm (20-24 inches) tall, with a finely divided, fibrous root system that may reach a depth of about 30 cm (12 inches). When environmental conditions are poor and/or when grazing animals crop the plants, downy brome plants that reach heights of just 5-10 cm (2-4 in) can still flower and produce viable seed. The stems are erect, slender, and glabrous or may be slightly soft and hairy. The nodding, open panicles with moderately awned spikelets are very distinctive. The spikelets readily penetrate fur, socks and pants and its seeds may thus be widely dispersed by people and animals.

Drift Control Agent: An adjuvant used to reduce the fine particles in the spray pattern that are responsible for herbicide drift and crop injury.

Dry flowable: A granule formulation similar to a wettable powder, except that the active ingredient is formulated on a large particle (granule) instead of onto a ground powder. This type of formulation offers essentially the same advantages and disadvantages as wettable powder formulations. However, these formulations generally are more easily mixed and measured than wettable powders. Because they create less dust when handling, they cause less inhalation hazard to the applicator during pouring and mixing.

Emulsifiable concentrate: A liquid herbicide formulation that contains the active ingredient, one or more solvents, and an emulsifier that allows mixing with water. Formulations of this type are highly concentrated, relatively inexpensive per pound of active ingredient, and easy to handle, transport, and store. In addition, they require little agitation (will not settle out or separate) and are not abrasive to machinery or spraying equipment.

Emulsifier: A substance that promotes the suspension of one liquid in another. Emulsifiers are often used to disperse petroleum-based herbicides in water.

Extender: An adjuvant added to an herbicide to modify, dilute, or adulterate it.

Foaming agent: An adjuvant used to reduce foaming in a spray system so that pumps and nozzles can operate properly.

Fungicide: A chemical substance that destroys or inhibits the growth of fungi.

Germination-Arrest Factor (GAF): Generally, any factor that inhibits or arrests seed germination. In specific embodiments of the disclosure, the term “GAF” is used to denote a particular highly specific, effective, naturally occurring bioherbicide, identified as 2-amino-4-formylaminooxy-but-3-enoic acid (also named 4-formylaminooxyvinylglycine), that is an alternative to chemical herbicides (e.g., Diuron) for the control of grassy weeds. GAF was identified as the product of particular isolates of certain rhizobacteria (Pseudomonas species; see Example 2 and elsewhere herein), representative examples of which were obtained from Willamette Valley soils in Oregon. GAF irreversibly arrests the germination of the seeds of a number of grassy weeds and some cultivated grasses, for example, but not limited to, annual bluegrass, downy brome, jointed goatgrass, roughstalk bluegrass, rattail fescue, and cultivated perennial ryegrass, and tall fescue, in addition to both domestic and wild oats. Germination of the seeds of cereals (wheat and barley) and dicots is also inhibited by GAF, but these plant species are much less sensitive to GAF than the seeds of grass species.

GAF has a unique mode of action that distinguishes it from less selective inhibitors of seed germination. Sensitive seeds initiate germination in the presence of GAF but seedling development is irreversibly arrested immediately following emergence of the coleorhiza and plumule. Thus, sensitivity to GAF is both species-specific and developmentally specific. Established seedlings appear to have little if any sensitivity to GAF, offering unique opportunities for weed management in grass cropping systems. Furthermore, culture filtrates containing GAF can be diluted, for example, filtrates can be diluted two- or three-fold, or even as much as ten-fold, or even more with little or no loss of activity. Thus, GAF is active at very low absolute concentrations.

An “effective amount” of GAF refers to an amount of the specific compound, or a derivative or modification thereof, that has an adverse biological effect on at least some of the seeds exposed to the GAF. For example, the effective amount of GAF may be an amount sufficient to arrest germination of at least some weed seeds in a seed population. In specific embodiments of the disclosure, an effective amount of GAF arrests germination in at least 10% of the seeds treated. In particular embodiments of the disclosure, an effective amount of GAF (or a related compound) arrests germination in at least 10%, at least 20%, or even 50% or more, of a seed population. In more particular embodiments of the disclosure, an effective amount of GAF arrests germination in over 90%, or nearly 100% of a seed population. Specific examples of effective amounts of GAF are provided in the Examples below.

Grassy Weed: Any of various undesirable plants having slender leaves characteristic of the grass family, for example, but not limited to crabgrass, goosegrass, dallisgrass, bahiagrass, annual bluegrass, downy brome, jointed goatgrass, roughstalk bluegrass, rattail fescue, perennial ryegrass, and tall fescue. Grassy weeds are troublesome in lawns, sports fields, playgrounds, golf courses and other turf grass areas because they are unsightly and lead to poor playing conditions. In addition, grassy weeds greatly increase mowing costs and present a safety hazard on sports fields and playgrounds.

Granule: An herbicide formulation that is used for soil-applied herbicides. In a granule formulation, the active ingredient is formulated onto large particles (granules). The primary advantages of this type of formulation are that the formulation is ready to use with simple application equipment (seeders or spreaders), and the drift potential is low because the particles are large and settle quickly. The disadvantages of this formulation are that it does not adhere to foliage (not intended for foliar applications), and may require mixing into the soil in order to achieve adequate herbicidal activity.

Herbicide: A compound or composition that produces an adverse effect on a plant, including (but not limited to) physiological damage to the plant; inhibition or modulation of plant growth; inhibition or modulation of plant reproduction; or death of the plant. Exemplary examples of herbicides include, but are not limited to, photosystem II inhibitors, protox inhibitors and superoxide generators, glufosinate, cell growth disruptors, cell growth inhibitors, lipid biosynthesis inhibitors, growth regulator herbicides, pigment inhibitors, inhibitors of amino acid biosynthesis, and inhibitors of cell wall biosynthesis.

To be effective, herbicides generally must 1) adequately contact plants; 2) be absorbed by plants; 3) move within the plants to the site of action, without being deactivated; and 4) reach toxic levels at the site of action. The application method used, whether preplant incorporated, preemergence, or postemergence, determines whether the herbicide will contact germinating seedlings, roots, shoots, or leaves of plants.

The term “mode of action” refers to the sequence of events from absorption into plants to plant death. The mode of action of the herbicide influences how the herbicide is applied. For example, contact herbicides that disrupt cell membranes, such as acifluorfen (Blazer) or paraquat (Gramoxone Extra), need to be applied postemergence to leaf tissue in order to be effective. Seedling growth inhibitors, such as trifluralin (Treflan) and alachlor (Lasso), need to be applied to the soil to effectively control newly germinated seedlings.

Because the seeds of many weed species are quite small and germinate within 1 to 2.5 cm (0.5 to 1.0 inch) of the soil surface, soil-applied herbicides generally are positioned in the top 2.5 to 5 cm (1 to 2 inches) of soil. Herbicide positioning can be accomplished by mechanical incorporation or by rainfall. Once an herbicide comes into contact with a plant, absorption through the roots or shoots occurs. An herbicide that is absorbed through the roots generally will be taken up as long as the herbicide-treated soil remains in contact with the absorbing region near the root tips. As the roots grow to greater soil depths, herbicide uptake generally declines. Therefore, weeds not killed before the root tips grow out of the herbicide-treated soil likely will survive.

Many soil-applied herbicides are absorbed through plant shoots while they are still underground and may kill or injure the shoots before they emerge from the soil. Volatile herbicides such as the thiocarbamates (e.g., EPTC [Eradicane]) and the dinitroanilines (e.g., trifluralin [Treflan]) can penetrate the shoot as gases. Less volatile herbicides such as the acetanilides (e.g., alachlor [Lasso]) are absorbed into the shoot as liquids. Physical and environmental factors that promote rapid crop emergence reduce the length of time that a plant is in contact with a soil-applied herbicide and, therefore, reduce the possibility of crop injury.

Herbicides differ in their ability to translocate within a plant. The soil-applied dinitroaniline herbicides (e.g., trifluralin [Treflan]) are not mobile within the plant. Therefore, their injury symptoms are confined to the site of uptake. Other herbicides are mobile within the plant. For example, soil-applied atrazine is absorbed by plant roots and moves upward within the xylem of the plant to be concentrated in the leaves. In general, injury symptoms will be most prominent at the site where the mobile herbicides concentrate.

Effective postemergence herbicide application is dependent upon adequate contact with above-ground plant shoots and leaves. Therefore, spray pressure, nozzle type, and volume generally are adjusted for adequate plant coverage.

For postemergence herbicides, the chemical and physical relationships between the leaf surface and the herbicide often determine the rate and amount of uptake. Factors such as plant size and age, water stress, air temperature, relative humidity, and herbicide additives can influence the rate and amount of herbicide uptake. Additives such as crop oil concentrates, surfactants, or liquid fertilizer solutions (e.g., UAN) can increase herbicide uptake by a plant. Application of herbicides under hot and dry conditions or application to older and larger weeds or weeds under water stress can decrease the amount of herbicide uptake. Differences in the rate and amount of herbicide uptake influence the potential for crop injury and weed control and often explain the year to year variation in the effectiveness of the herbicide. In addition, the faster an herbicide is absorbed by a plant, the less likely it will be that rain will wash the herbicide off the plants.

Like soil-applied herbicides, postemergence herbicides differ in their ability to move within a plant. For adequate weed control, nonmobile postemergence herbicides generally must thoroughly cover the plant. Nonmobile herbicides are often called contact herbicides, and include the bipyridylium, diphenylether, benzothiadiazole, and nitrile families. Other herbicides are mobile within the plant and can move from the site of application to their site of herbicidal activity. For example, growth regulator herbicides such as 2,4-D and dicamba (Banvel) move both upward and downward within a plant's phloem to the growing points of the shoots and roots. In general, injury symptoms will be most prominent at the sites at which the mobile herbicides concentrate.

Plants that can rapidly degrade or deactivate an herbicide can escape that herbicide's toxic effects. For example, corn is tolerant to the triazine herbicides because it quickly deactivates these herbicides by binding them to naturally occurring plant chemicals. Soybean tolerance to metribuzin (Sencor, Lexone) is at least partially due to the deactivation of the herbicide by conjugating to plant sugar molecules.

Furthermore, a crop may be injured by an herbicide to which it is normally tolerant. This often occurs because environmental stresses such as hot or cold temperatures, high relative humidity, or hail decrease a plant's natural ability to reduce herbicide uptake or deactivate an herbicide. Postemergence cyanazine (Bladex) injury to corn under cold and wet weather conditions is an example of environmentally induced herbicide injury. An excessive application of herbicide, due to misapplication, can also injure a tolerant crop by overwhelming the crop's herbicide degradation and deactivation systems.

A number of weed species that were once susceptible and easily managed by certain herbicides have now developed resistance to these agents. These weeds are no longer controlled by applications of previously effective herbicides. To date, at least 53 species of weeds are resistant to at least five different herbicide families.

Invert emulsifier: An adjuvant that allows water-based herbicides to mix with a petroleum carrier.

Isolated: An isolated biological agent or component has been substantially separated, produced apart from, or purified away from other biological agents or components in the environment or from a cell of the organism in which the agent or component naturally occurs. In some examples, an isolated biological agent or component is a nucleic acid, protein or modified amino acid that has been isolated and purified by standard purification methods. In other examples, it is an isolated bacterium that has been identified and cultured to homogeneity or near homogeneity by standard microbiological techniques. The term also embraces biochemicals prepared by recombinant expression in a host cell as well as those that have been chemically synthesized extrabiologically. The terms isolated and purified do not require absolute purity; rather, they are intended as relative terms. Thus, for example, an isolated modified amino acid preparation is one in which the modified amino acid is more enriched than in its natural environment within a cell. Preferably, an isolated preparation is purified such that the modified amino acid is at least 50% of the total biochemical content of the preparation.

Jointed Goatgrass (Aegilops cylindrica): A native of southern Europe and western Asia, jointed goatgrass is so closely related to wheat that both species can interbreed. It is difficult to distinguish jointed goatgrass from wheat until spikes appear. Jointed goatgrass spreads exclusively by seed, and grows best in cultivated fields, but can also invade grasslands.

Jointed goatgrass is a winter annual, but about five percent of a population may be spring annuals. Leaves are grasslike, up to a half-inch wide, and have evenly spaced fine hairs along the leaf edges and down the sheath opening. The ligule is short and membranous; auricles are short and hairy. Stems can grow up to four feet tall and are tipped with slender, cylindrical spikes that appear to be a series of joints stacked on top of each other. Reddish to straw-colored spikes emerge in May to June, and uppermost joints are tipped by straight awns. Up to three seeds are enclosed in each joint.

Jointed goatgrass is found in all major United States winter wheat production regions, from Texas to South Dakota and eastern Montana, and in portions of the Northwest and Utah. No herbicides are available that can selectively control jointed goatgrass in winter wheat, however spring tillage and general grass killers provide good control.

Liquid flowable: An herbicide formulation made up of finely ground active ingredient suspended in a liquid. Flowables generally are mixed with water for application, are easily handled and applied, and seldom clog nozzles. Some of their disadvantages are that they may leave a visible residue on plant and soil surfaces, and typically require constant and thorough agitation to remain in suspension.

Nematicide: A substance or preparation used to kill nematodes.

Ninhydrin: A chemical compound with the molecular formula C₉H₆O₄, ninhydrin is also known as ninhydrin monohydrate, 1,2,3-triketohydrindene monohydrate, 1,2,3-indantrione monohydrate, 2,2-dihydroxy-1,3-indandione, 1H-indene-1,2,3-trione monohydrate. Ninhydrin produces a purple reaction product in the presence of primary amines. A ninhydrin-positive or ninhydrin-reactive agent is one that produces such a reaction product, and the presence of such a reaction product indicates that the agent includes at least one primary amine. Thus, a ninhydrin-reactive agent is one that includes at least one primary amine, for example, an amino acid, peptide, protein, or another agent that includes at least one primary amine, such as an enzymatically synthesized or modified agent.

Pellet: An herbicide formulation used for soil-applied herbicides. In a pellet formulation, the active ingredient is formulated onto large particles (pellets). The primary advantages of this type of formulation are that the formulation is ready to use with simple application equipment (seeders or spreaders), and the drift potential is low because the particles are large and settle quickly. The disadvantages of this formulation are that it does not adhere to foliage (not intended for foliar applications), and may require mixing into the soil in order to achieve adequate herbicidal activity.

Penetrant: An adjuvant that allows an herbicide to get through the outer surface to the inside of a treated area.

Perennial Ryegrass (Lolium perenne): A perennial grass that grows to 8-90 centimeters tall, and has loose to densely tufted, short-lived, glabrous leaves. Perennial ryegrass can be decumbent or (rarely) prostrate, sometimes rooting at lowest nodes, usually with two to four nodes below the spike. Basal leaf-sheaths are green, reddish, purplish, or straw-colored, and leaf-blades are folded in young shoots. Mature blades are acute, attenuate, or somewhat rounded at apices, usually less than 14 centimeters long, and one to six millimeters broad. Auricles typically are present, but may be absent, and are minute, up to three millimeters long; bearing 5-37 spikelets.

Pesta: A granular product made from cereal grain flour and a biocontrol agent. The process encapsulates biocontrol agents in pasta-like products called pesta (U.S. Pat. No. 5,074,902; and Connick et al., 1991). Bacteria formulated in such media may exhibit extended shelf and field-life (e.g., Connick et al., Am. Biotechnol. Lab. 14:34-37, 1996; and Connick et al., 1998). These characteristics are desired in a product which may be stored prior to use or shipped over long-distances prior to being used for weed control in a field. Therefore, the biocontrol compositions of the present invention may be formulated in a suitable composition, for example, but not limited to, pesta.

Pesticide: A chemical used to kill pests, for example insects.

Preservative: An adjuvant that inhibits degradation of an herbicide.

Purified: The term purified does not require absolute purity; rather, it is intended as a relative term. Thus, for example, a purified protein preparation is one in which the protein referred to is more pure than the protein in its natural environment within a cell.

Rattail Fescue (Vulpia myuros): Rattail fescue is an annual grassy weed. It grows to be up to about 60 cm (two feet) tall. Rattail fescue exhibits narrow leaf blades, which are folded and hairless. Panicles are slender and up to eight inches in length, and awns are about 5/16 to ⅜ inch long.

Roughstalk Bluegrass (Poa trivialis): A perennial bluegrass that grows from stolons that may reach about 30 to 90 cm (1 to 3 feet) in height. These plants go dormant throughout the summer and carry out their life cycle during the winter months.

Roughstalk bluegrass stems are covered with many small hairs, with brown to purple bands surrounding the nodes. Leaves are folded in the bud and have the boat-shaped tip typical of most bluegrass species. Leaf blades are about 5 to 17 cm (2 to 7 inches) long, and about two to five millimeters wide. They are covered with many small hairs, and have a relatively large (about four to six millimeters) membranous ligule. The seedhead is a panicle very similar to other bluegrass turf species.

Roughstalk bluegrass produces fibrous roots with a stoloniferous system that contributes significantly to the spread of this weed. As an agronomic weed, roughstalk bluegrass is relatively easy to distinguish from other grasses. The distinctive boat-shaped leaf tip, seedhead, growing season, and presence of stolons are all characteristics that aid in identification.

Safener: An adjuvant that reduces the toxicity of an herbicide formulation to the herbicide handler or to the crop.

Soluble powder: An herbicide formulation that, when mixed with water, dissolves readily and forms a true solution. Soluble powder formulations are dry and include the active ingredient and additives. When thoroughly mixed, no further agitation is necessary to keep the active ingredient dissolved in solution.

Solution: A liquid formulation that includes an active ingredient and an additive. Solution formulations are designed for those active ingredients that dissolve readily in water. Generally, when herbicides formulated as solutions are mixed with water, the active ingredient will not settle out of solution or separate.

Solvent: A substance (usually liquid) suitable for, or employed in solution, or in dissolving something. For example, water is an appropriate solvent of most salts; alcohol of resins; ether of fats; and mercury or acids of metals.

Spreader: An adjuvant that allows an herbicide to form a uniform coating layer over the treated surface.

Stabilizer: A substance that renders or maintains a solution, mixture, suspension, or state resistant to chemical change.

Sticker: An adjuvant that causes an herbicide to adhere to plant foliage. Stickers reduce spray runoff during application and washoff by rain. Many stickers are blended with wetting agents so that they both increase spray coverage and provide better adhesion. These combined products often are call spreader-stickers.

Surfactant: A type of adjuvant designed to improve the dispersing/emulsifying, absorbing, spreading, sticking and/or pest-penetrating properties of an herbicide formulation. Surfactants can be divided into the following five groupings: 1) non-ionic surfactants, 2) crop oil concentrates, 3) nitrogen-surfactant blends, 4) esterified seed oils, and 5) organo-silicones.

Suitable surfactants may be nonionic, cationic, or anionic, depending on the nature of the compound used as an active ingredient. Surfactants may be mixed together in some embodiments of the disclosure. Nonionic surfactants include polyglycol ether derivatives of aliphatic or cycloaliphatic alcohols, saturated or unsaturated fatty acids and alkylphenols. Fatty acid esters of polyoxyethylene sorbitan, such as polyoxyethylene sorbitan trioleate, also are suitable nonionic surfactants. Other suitable nonionic surfactants include water-soluble polyadducts of polyethylene oxide with polypropylene glycol, ethylenediaminopolypropylene glycol and alkylpolypropylene glycol. Particular nonionic surfactants include nonylphenol polyethoxyethanols, polyethoxylated castor oil, polyadducts of polypropylene and polyethylene oxide, tributylphenol polyethoxylate, polyethylene glycol and octylphenol polyethoxylate.

Cationic surfactants include quaternary ammonium salts carrying, as N-substituents, an 8 to 22 carbon straight or branched chain alkyl radical. The quaternary ammonium salts may carry additional N-substituents, such as unsubstituted or halogenated lower alkyl, benzyl, or hydroxy-lower alkyl radicals. Some such salts exist in the form of halides, methyl sulfates, and ethyl sulfates. Particular salts include stearyldimethylammonium chloride and benzyl bis(2-chloroethyl)ethylammonium bromide.

Suitable anionic surfactants may be water-soluble soaps as well as water-soluble synthetic surface-active compounds. Suitable soaps include alkali metal salts, alkaline earth metal salts, and unsubstituted or substituted ammonium salts of higher fatty acids. Particular soaps include the sodium or potassium salts of oleic or stearic acid, or of natural fatty acid mixtures. Synthetic anionic surfactants include fatty sulfonates, fatty sulfates, sulfonated benzimidazole derivatives, and alkylarylsulfonates. Particular synthetic anionic surfactants include the sodium or calcium salt of ligninsulfonic acid, of dodecyl sulfate, or of a mixture of fatty alcohol sulfates obtained from natural fatty acids. Additional examples include alkylarylsulfonates, such as sodium or calcium salts of dodecylbenzenesulfonic acid, or dibutylnaphthalenesulfonic acid. Corresponding phosphates for such anionic surfactants are also suitable.

Tall Fescue (Festuca arundinacea): Tall fescue is a deep rooted, cool season perennial grass. The plant produces vigorous growth in the spring and fall and its extensive root system helps it withstand drought conditions. Tall fescue produces short rhizomes but it has a bunch-type growth habit; and spreads primarily by erect tillers. Individual tillers, or stems, terminate in an inflorescence, reach about 1 to 1.3 meters (3 to 4 feet) in height, and have broad, dark green basal leaves. Leaf blades are glossy on the underside and serrated on the margins. The leaf sheath is smooth and the ligule is a short membrane. The inflorescence is a compact panicle, about 7 to 10 cm (3 to 4 inches) long with lanceolate spikelets about one-half inch or more in length. The grass flowers in the spring and seed mature in early summer.

Tall fescue is found from the Pacific Northwest to the southern states in low-lying pastures. Although it grows best in moist environments, tall fescue has good drought tolerance and will survive during dry periods in a dormant state. Tall fescue is adapted to a wide range of soils, but does best on clay soils high in organic matter. Tall fescue is well adapted to the “transition zone” of the United States where summers are too hot and humid for cool season grasses and winters too cold for warm season grasses. In the South, tall fescue is best adapted to those states in the transition zone: Oklahoma, Arkansas, Missouri, Tennessee, Kentucky, Virginia and northern parts of North Carolina, Georgia and Texas.

Thickener: An adjuvant that reduces drift by increasing droplet size and reducing volume of spray contained in drift-prone droplets.

Timed-Release Coating: A coating on a solid or particulate formulation that retards degradation and prolongs GAF activity. Coatings can be divided into three categories: (1) coatings that directly degrade in the presence of water, (2) coatings that are broken apart by wet and dry cycles, and (3) coatings degraded by specific temperatures, for example Degree Herbicide® by Monsanto.

Water-Resistant Coating: A coating on a solid or particulate GAF formulation that repels water and delays dissolution of the herbicide. One common technique used commercially is interfacial polycondensation of multifunctional isocyanates with multifunctional amines. In this technique, the oil phase containing the active agene and the isocyanate is emulsified in the aqueous phase containing the amine monomer. The isocyanate reacts with the amine at the oil-water interface to form a solid polyurea shell wall about the encapsulated active agent. A second technique involves coating GAF with whey protein that has been treated to provide a specific form or structure that is more highly resistant to dissolution in water.

Weed: Any unsightly, useless, or injurious plant, particularly plants growing in cultivated ground to the injury of the crop or desired vegetation, plant seeds contaminating a desired seed variety, or plants growing to the disfigurement of an area, for example a garden or a road-side. The term “weed” has no definite application to any particular plant, or species of plants. In some embodiments, a weed is a grassy weed, for instance, annual bluegrass, downy brome, jointed goatgrass, roughstalk bluegrass, rattail fescue, perennial ryegrass, tall fescue, or domestic or wild oats.

Wettable powder: A dry, finely ground herbicide formulation in which the active ingredient is combined with a finely ground carrier (usually mineral clay), along with other ingredients to enhance the ability of the powder to suspend in water. Generally, the powder is mixed with water for application. Wettable powders are one of the most widely used herbicide formulations and offer low cost and ease of storage, transport, and handling; lower phytotoxicity potential than emulsifiable concentrates and other liquid formulations; and less skin and eye absorption hazard than emulsifiable concentrates and other liquid formulations. Some disadvantages are that they require constant and thorough agitation in the spray tank, are abrasive to pumps and nozzles (causing premature wear), may produce visible residues on plant and soil surfaces, and can create an inhalation hazard to the applicator while handling (pouring and mixing) the concentrated powder. Typical solid diluents are described in Watkins et al., Handbook of Insecticide Dust Diluents and Carriers, 2nd Ed., Dorland Books, Caldwell, N.J. The more absorptive diluents are preferred for wettable powders and the denser ones for dusts.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” In case of conflict, the present specification, including explanations of terms, will control. In addition, the materials, methods, and examples are illustrative only and not intended to be limiting.

### III. Description of Several Embodiments

Provided herein in a first embodiment is an isolated Germination-Arrest Factor produced by a strain of bacteria or by chemical synthesis, wherein the isolated Germination-Arrest Factor comprises a vinylglycine molecule that inhibits or arrests the germination of grassy weed seeds. In representative examples thereof, the isolated Germination-Arrest Factor is produced by a bacterium, wherein the genome of the bacterium comprises a formyl-transferase gene having a sequence at least 80% identical to the sequence set forth as SEQ ID NO: 2. In other specific examples, the bacterium is selected from the group consisting of Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, and Enterobacter kobei A3203.

In a particular embodiment, the isolated Germination-Arrest Factor is 2-amino-4-formylaminooxy-but-3-enoic acid (also named 4-formylaminooxyvinylglycine).

Also provided are structural modifications and derivatives of the GAF molecules described herein. In some embodiments of such modifications or derivatives of the Germination-Arrest Factor, the vinylglycine molecule has the structure of one of Formulae IA, IB or IC, and is effective to inhibit or arrest weed seed germination or for other plant growth regulation:

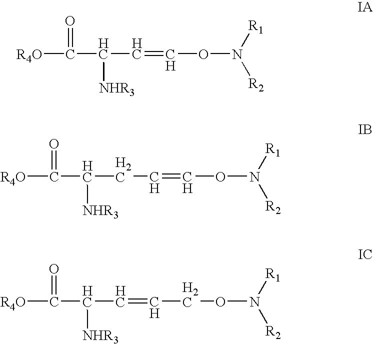

wherein R₁ and R₂ independently are selected from H, optionally substituted lower aliphatic, optionally substituted amino, alkoxy, hydroxy, —COOH; hydroxy alkyl, alkyl amino; R₃ independently is selected from H, acetyl, propanyl, and optionally substituted lower aliphatic; and R₄ independently is selected from H and optionally substituted lower aliphatic.

Other example embodiments are modifications and derivatives of the Germination-Arrest Factor, wherein the vinylglycine molecule has the structure of one of Formulae IIA, IIB or IIC, and is effective to inhibit or arrest weed seed germination

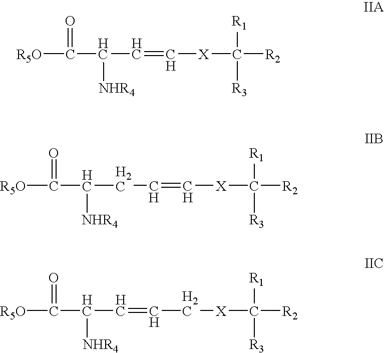

wherein X is selected from O, S, and optionally substituted amino; R₁, R₂, and R₃ independently area selected from H, optionally substituted lower aliphatic, optionally substituted amino, alkoxy, hydroxy, —COOH, —NHCOOH, hydroxy alkyl, alkyl amino; R₄ independently is selected from H, acetyl, propanyl and optionally substituted lower aliphatic; and R₅ independently is selected from H and optionally substituted lower aliphatic.

Other example embodiments are modifications and derivatives of the Germination-Arrest Factor, wherein the molecule has the structure of Formula III and is effective to inhibit or arrest weed seed germination.

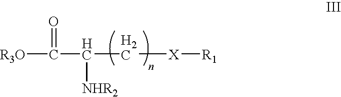

wherein X is selected from O, S and optionally substituted amino; n is one, two, three, four, or five; R₁ independently is selected from optionally substituted lower aliphatic, optionally substituted amino, alkoxy, hydroxy, —NHCOOH; hydroxy alkyl, alkyl amino; R₂ independently is selected from H, acetyl, propanyl, and optionally substituted lower aliphatic; and R₃ independently is selected from H and optionally substituted lower aliphatic.

In yet other examples of structural modifications and derivatives of the Germination-Arrest Factor, the vinylglycine molecule has the structure of Formula IV and is effective to inhibit or arrest weed seed germination:

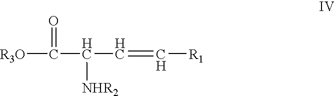

wherein
R₁═NHOH, R₂═H, CH₃, COCH₃, R₃═H, CH₃, CH₃CH₂
R₁═NHOCH₃, R₂═H, CH₃, COCH₃, R₃═H, CH₃, CH₃CH₂
R₁═OCH₂CH₂NH₂, R₂═H, CH₃, COCH₃, R₃═H, CH₃, CH₃CH₂
   R₁═OCH₃, R₂═H, CH₃, COCH₃, R₃═H, CH₃, CH₃CH₂
   R₁═ONH₂, R₂═H, CH₃, COCH₃, R₃═H, CH₃, CH₃CH₂
   R₁═CH₂PO₃²⁻, R₂═H, CH₃, COCH₃, R₃═H, CH₃, CH₃CH₂
   R₁═CH₂CH₂CO₂H, R₂═H, CH₃, COCH₃, R₃═H, CH₃, CH₃CH₂
   R₁═CHCH₂, R₂═H, CH₃, COCH₃, R₃═H, CH₃, CH₃CH₂
   R₁═CH₂NHCONH₂, R₂═H, CH₃, COCH₃, R₃═H, CH₃, CH₃CH₂

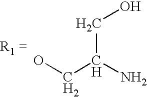

### R₂═H, CH₃, COCH₃, R₃═H, CH₃, CH₃CH₂

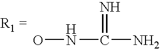

### R₂═H, CH₃, COCH₃, R₃═H, CH₃, CH₃CH₂

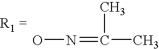

### R₂═H, CH₃, COCH₃, R₃═H, CH₃, CH₃CH₂

or wherein the compositions include alkyloxy and aryloxy vinylglycines, including those wherein

R₁═OCH₂CH(CH₃)CH₃, SCH₃, R₂═H, CH₃, COCH₃, R₃═H, CH₃, CH₃CH₂

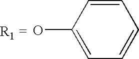

### R₂═H, CH₃, COCH₃, R₃═H, CH₃, CH₃CH₂

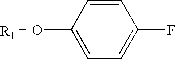

### R₂═H, CH₃, COCH₃, R₃═H, CH₃, CH₃CH₂

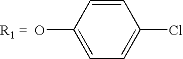

### R₂═H, CH₃, COCH₃, R₃═H, CH₃, CH₃CH₂

In yet other examples of structural modifications and derivatives of the Germination-Arrest Factor, the vinylglycine molecule is effective to inhibit weed seed germination and has the formula

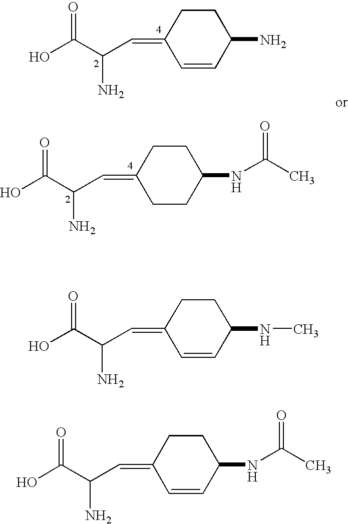

In yet further structural modifications and derivatives of the Germination-Arrest Factor, the vinylglycine molecule is effective to inhibit weed seed germination and has the structure of Formula V:

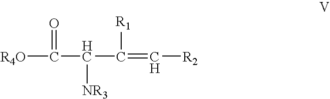

wherein the substituents R₁ and R₂ independently are selected from optionally substituted lower aliphatic and halo, R₃ is selected from H, lower alkyl, acetyl and propanyl, and R₄ is selected from H and lower alkyl.

Optionally, in any of the compounds described above the carboxyl group is replaced by a sulfonic acid group and the resulting compound inhibits weed seed germination. In particular embodiments, the sulfonic acid group replaces the carboxyl group at substituents R₁ and/or R₂. In other embodiments, the sulfonic acid group replaces any carboxyl group at position 1.

In various embodiments, the Germination-Arrest Factor or the structural modifications or derivatives of the Germination-Arrest Factor described herein are effective to inhibit germination of one or more of Poa annua, Poa trivialis or Bromus tectorum. In other examples, the Germination-Arrest Factor or structurally related molecules such as structural modifications or derivatives thereof, are effective to control weeds selected from crabgrass, goosegrass, dallisgrass, bahiagrass, annual bluegrass, downy brome, jointed goatgrass, roughstalk bluegrass, rattail fescue, perennial ryegrass, or tall fescue.

Another embodiment is a method for inhibiting or arresting weed germination in a growth medium in which it would be desirable to inhibit or arrest weed germination, the method comprising applying at least one bacterial strain that produces a vinylglycine to the growth medium in an amount sufficient to inhibit or arrest weed germination. In some examples, the bacterial strain is a Pseudomonas. In some cases, the bacterial strain has a genome comprising a formyl-transferase gene having a sequence at least 80% identical to the sequence set forth as SEQ ID NO: 2. In other example methods for inhibiting or arresting weed germination the bacterial strain is selected from the group consisting of Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, and Enterobacter kobei A3203, and mixtures of two or more thereof.

Yet another embodiment provides a method for inhibiting or arresting weed germination in a growth medium in which it would be desirable to inhibit or arrest weed germination, the method comprising applying an isolated Germination-Arrest Factor or a structural modification or derivative of the Germination-Arrest Factor as described herein to the growth medium in an amount sufficient to inhibit or arrest grassy weed germination. In some examples the isolated Germination Arrest Factor is produced by a bacterial strain that has a genome comprising a formyl-transferase gene having a sequence at least 80% identical to the sequence set forth as SEQ ID NO: 2. In other examples, the bacterial strain is selected from the group consisting of Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, and Enterobacter kobei A3203. In still other examples, the Germination-Arrest Factor or structural modification or derivative thereof is optionally applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide. Alternatively, the active ingredient is applied in a formulation that is a solution, a soluble powder, an emulsifiable concentrate, a wettable powder, a liquid flowable, a dry flowable, a water-dispersible granule, a granule, or a pellet.

Also provided are methods of inhibiting or arresting weed germination among seeds that are insensitive to the Germination-Arrest Factor, the method comprising applying the isolated Germination-Arrest Factor, or any of the structural derivatives described herein, to the GAF-insensitive seeds in an amount sufficient to inhibit or arrest germination of any grassy weed seeds that are mixed in with the GAF-insensitive seeds. In examples of such methods, the Germination-Arrest Factor or the structural modification or derivative thereof is applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide. In further examples, the active ingredient is applied in a formulation that is a solution, a soluble powder, an emulsifiable concentrate, a wettable powder, a liquid flowable, a dry flowable, a water-dispersible granule, a granule, or a pellet.

Also provided is a composition for inhibiting or arresting the germination of weeds, the composition comprising Germination-Arrest Factor, or at least one structurally related compound such as a structural modification or derivative of the Germination-Arrest Factor; and a timed- or temperature-release coating over at least a portion of the Germination-Arrest Factor, structurally related compound, or modification or derivative thereof. Optionally, the composition further comprises a water-resistant coating over the timed-or temperature-release coating.

In another embodiment, there is provided a method of inhibiting or arresting weed germination in an area in which inhibiting or arresting weed germination is desirable, the method comprising broadcasting an herbicidally effective amount of Germination-Arrest Factor or any structurally related compound or derivative thereof, at least once a year across the area, thereby inhibiting or arresting weed germination in the area. By way of example, the area is a grass patch, an agricultural field, a natural landscape, or a road-side.

In examples of the method of inhibiting or arresting weed germination in an area in which inhibiting or arresting weed germination is desirable, the Germination-Arrest Factor or structurally related compound or derivative thereof is applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide. Optionally, the active ingredient is applied in a formulation that is a solution, a soluble powder, an emulsifiable concentrate, a wettable powder, a liquid flowable, a dry flowable, a water-dispersible granule, a granule, or a pellet.

In any of the described methods, the Germination-Arrest Factor or structurally related compound or derivative of Germination-Arrest Factor is formulated as a granule. Optionally, the granule is at least partially coated with a timed-or temperature-release coating, such as a water-resistant coating. Further, any of the described methods may be in certain embodiments a method of inhibiting grassy weeds among dicot species or among established monocot seedlings.

Also provided are methods of producing GAF, the methods comprising culturing a bacterium in a suitable culture medium wherein the genome of the bacterium comprises a formyl-transferase gene having a sequence at least 80% identical to the sequence set forth as SEQ ID NO: 2; collecting the culture medium; and purifying the culture medium to produce the isolated Germination-Arrest Factor. In examples of such methods, the bacterium is Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, or Enterobacter kobei A3203.

In other embodiments, there is provided a method of producing the isolated Germination-Arrest Factor, the method comprising culturing a genetically engineered bacterium in a suitable culture medium; collecting the culture medium; and purifying the culture medium to produce the isolated Germination-Arrest Factor. In examples of this method, the strain of bacteria is genetically engineered to comprise a genetic sequence that comprises a formyl-transferase gene having a sequence at least 80% identical to the sequence set forth as SEQ ID NO: 2.

Other embodiments are kits for inhibiting or arresting weed germination or growth, the kits comprising an isolated Germination-Arrest Factor or structurally related compound or derivative thereof as provided herein; and a container. Optionally, the kit is accompanied by instructions for using the kit.

The isolated Germination-Arrest Factor described herein, as well as the structural modifications and derivatives of the isolatedGermination-Arrest Factor described herein, can be used as seed-cleaning adjuvants in seed-cleaning processes, for instance as a supplement or alternative to physical removal of target weed seeds.

In yet other embodiments of the methods described herein, a bacterial strain that produces a vinylglycine is substituted for the Germination-Arrest Factor or structurally related compound or derivative of GAF. For instance, the bacterial strain may be Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, or Enterobacter kobei A3203. More generally, the bacterial strain comprises a formyl-transferase gene having a sequence at least 80% identical to the sequence set forth as SEQ ID NO: 2.

In further examples of the methods provided herein, an inhibitor of pyridoxal phosphate-dependent enzyme reactions other than a vinylglycine is substituted for the Germination-Arrest Factor. For instance, the inhibitor in some embodiments is aminooxyacetic acid.

### IV. Germination-Arrest Factor (GAF)

Disclosed herein is a highly specific, effective, naturally-occurring bioherbicide. As described in Banowetz et al. (Biological Control, 46: 380-390, 2008; which is hereby incorporated by reference), GAF is an alternative to synthetic chemical herbicides (e.g., Diuron) for the control of grassy weeds or other types of weeds that propagate by seeds that are sensitive to GAF. For example, in one embodiment of the disclosure, GAF is used in grass seed production systems or other cropping systems. In other embodiments of the disclosure, GAF is used in natural settings, in horticultural landscapes, or in other settings where grassy weeds are problematic.

GAF was identified as the product of particular isolates of certain rhizobacteria (Pseudomonas species; see Example 5) obtained from Willamette Valley soils in Oregon. GAF irreversibly arrests the germination of the seeds of a number of grassy weeds, for example, but not limited to, annual bluegrass, downy brome, jointed goatgrass, roughstalk bluegrass, and rattail fescue, perennial rye grass, and tall fescue (which, in some embodiments of the disclosure, might fall to the ground during harvest of these crop plants), in addition to both domestic and wild oats.

GAF has a unique mode of action that distinguishes it from less selective inhibitors of seed germination. Sensitive seeds initiate germination in the presence of GAF, but seedling development is irreversibly arrested immediately following emergence of the coleorhiza and plumule. Thus, sensitivity to GAF is both species-specific and developmentally specific. Established seedlings appear to have little if any sensitivity to GAF, offering unique opportunities for weed management in grass cropping systems. Furthermore, culture filtrates containing GAF can be diluted, for example, filtrates can be diluted two- or three-fold, or even ten-fold, or more, with little or no loss of activity. Thus, GAF is active at very low absolute concentrations.

Further characterization of GAF described its size and solubility, as well as the stability of its activity over time and under varying temperature and pH conditions (see Banowetz et al., Biological Control, 50: 103-110, 2009; which is hereby incorporated by reference). GAF is a small hydrophilic molecule that is insoluble or sparingly soluble in organic solvents. GAF activity in culture filtrates stored at 4° C. diminishes over time, with 10% activity remaining after 17 months. Additionally, GAF remains active at temperatures up to 65° C. and under basic as well as acidic conditions if exposures to these conditions are limited to a few hours time.

Elsewhere (see Armstrong et al., Biological Control, 51: 181-190, 2009, hereby incorporated by reference), it was shown that GAF reacts with ninhydrin, and contains at least one ionizable group. GAF chromatographs with defined R_{f} values in particular thin-layer chromatography systems, and mutation of any of the two genes that have been identified as essential for GAF biosynthesis results in both loss of the biological activity associated with GAF and loss of one particular ninhydrin-positive band visible in these TLC separations. As described herein, purification and analysis of the compound responsible for GAF activity has identified GAF as 2(R/S)-amino-4-formylaminooxy-but-3-enoic acid (i.e., 4-formylamino-D/L-oxyvinylglycine).

### V. Structural Modifications of the Germination-Arrest Factor (GAF)

The present disclosure includes GAF (i.e. 2(R/S)-amino-4-formylaminooxy-but-3-enoic acid) and structurally related, biologically active molecules that mimic the action of GAF in inhibiting the germination of gassy weeds. The present disclosure includes synthetic embodiments of the naturally-occurring compound (2(R/S)-amino-4-formylaminooxy-but-3-enoic acid) described herein, as well as analogues, derivatives, and variants of this compound that inhibit or arrest weed seed germination if such compounds have not been used previously for this purpose. This disclosure includes both the use of these molecules to inhibit or arrest weed seed germination and their use as plant growth regulators for other plant growth regulating purposes if the specific analogue, derivative, and variant molecules described herein have not been utilized previously for such purposes.

This disclosure also includes a method of inhibiting the germination of grassy weeds with compounds that are not structurally related to GAF but share with vinylglycines the ability to block pyridoxal-phosphate dependent enzyme reactions.

GAF may be modified by a variety of chemical and enzymatic techniques to produce derivatives having essentially the same activity as the unmodified natural product (2-amino-4-formylaminooxy-but-3-enoic acid) and optionally having other desirable properties, such as changes in target specificity or enhanced efficacy. For example, various substituents may be introduced at the amino group or at the carboxyl group known to be present on the unmodified natural product.

A number of structural modifications or derivatives of the specific GAF molecule specifically contemplated for use in the methods and compositions disclosed herein include, without limitation, those of Formulae IA, IB and IC, and is effective to inhibit or arrest weed seed germination:

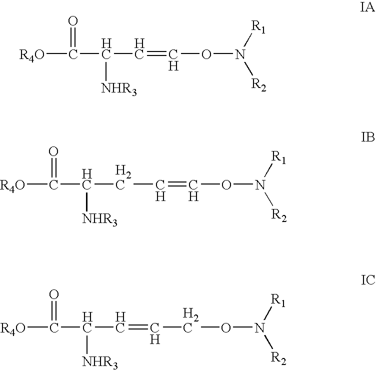

wherein R₁ and R₂ independently are selected from H, optionally substituted lower aliphatic, such as lower alkyl, optionally substituted amino, alkoxy, hydroxy, —COOH, hydroxy alkyl, alkyl amino and the like.

With continued reference to Formulae IA, IB and IC, R₃ independently is selected from H, and optionally substituted lower aliphatic, such as optionally substituted lower alkyl, acetyl and propanyl. In exemplary embodiments, R₃ is H, and in other embodiments R₃ is lower alkyl, such as methyl.

With continued reference to Formulae IA, IB and IC, R₄ independently is selected from H, and optionally substituted lower aliphatic, such as optionally substituted lower alkyl. In exemplary embodiments, R₄ is H, and in other embodiments R₄ is lower alkyl, such as methyl or ethyl.

Particular examples of compounds have Formulae IA, IB and IC, wherein R₁, R₂, R₃ and R₄ independently are as follows:
R₁═CH₂CH₃, R₂═H, CH₃, R₃═H, CH₃, COCH₃, R₄═H, CH₃, CH₃CH₂
R₁═CH₂CH₂CH₃, R₂═H, CH₃, R₃═H, CH₃, COCH₃, R₄═H, CH₃, CH₃CH₂
R₁═CH₂CH₂CH₂CH₃, R₂═H, CH₃, R₃═H, CH₃, COCH₃, R₄═H, CH₃, CH₃CH₂
R₁═CH₂NH₂, R₂═H, CH₃, R₃═H, CH₃, COCH₃, R₄═H, CH₃, CH₃CH₂
R₁═CH₂CH₂NH₂, R₂═H, CH₃, R₃═H, CH₃, COCH₃, R₄═H, CH₃, CH₃CH₂
R₁═CH₂CH₂CH₂NH₂, R₂═H, CH₃, R₃═H, CH₃, COCH₃, R₄═H, CH₃, CH₃CH₂
R₁═CH₂CH(CH₃)NH₂, R₂═H, CH₃, R₃═H, CH₃, COCH₃, R₄═H, CH₃, CH₃CH₂
R₁═CH₂CH(NH₂)CH₂NH₂, R₂═H, CH₃, R₃═H, CH₃, COCH₃, R₄═H, CH₃, CH₃CH₂
R₁═CH₂OH, R₂═H, CH₃, R₃═H, CH₃, COCH₃, R₄═H, CH₃, CH₃CH₂
R₁═CH₂CH₂OH, R₂═H, CH₃, R₃═H, CH₃, COCH₃, R₄═H, CH₃, CH₃CH₂
R₁═CH₂CH₂CH₂OH, R₂═H, CH₃, R₃═H, CH₃, COCH₃, R₄═H, CH₃, CH₃CH₂
R₁═CH₂CH(CH₃)OH, R₂═H, CH₃, R₃═H, CH₃, COCH₃, R₄═H, CH₃, CH₃CH₂
R₁═CH₂CH(OH)CH₂OH, R₂═H, CH₃, R₃═H, CH₃, COCH₃, R₄═H, CH₃, CH₃CH₂
R₁═CH₂CHO, R₂═H, CH₃, R₃═H, CH₃, COCH₃, R₄═H, CH₃, CH₃CH₂
R₁═CH₂CH₂CHO, R₂═H, CH₃, R₃═H, CH₃, COCH₃, R₄═H, CH₃, CH₃CH₂
R₁═CH₂COOH, R₂═H, CH₃, R₃═H, CH₃, COCH₃, R₄═H, CH₃, CH₃CH₂
R₁═CH₂CH₂COOH, R₂═H, CH₃, R₃═H, CH₃, COCH₃, R₄═H, CH₃, CH₃CH₂
R₁═COOH, R₂═H, CH₃, R₃═H, CH₃, COCH₃, R₄═H, CH₃, CH₃CH₂
R₁═COOCH₃, R₂═H, CH₃, R₃═H, CH₃, COCH₃, R₄═H, CH₃, CH₃CH₂

Additional structural modifications or derivatives of the GAF molecule specifically contemplated for use in the methods and compositions disclosed herein include, without limitation, those of Formulae IIA, IIB and IIC, and is effective to inhibit or arrest weed seed germination:

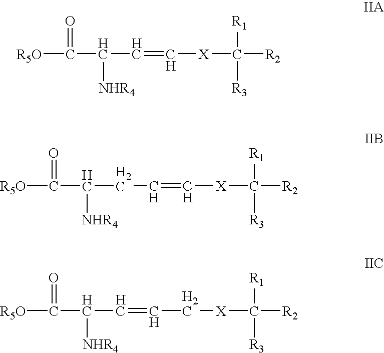

wherein the variable group X typically is selected from O, S and optionally substituted amino, and R₁, R₂ and R₃ independently are selected from H, optionally substituted lower aliphatic, such as lower alkyl, optionally substituted amino, alkoxy, hydroxy, —COOH, —NHCOOH, hydroxy alkyl, alkyl amino and the like.

With continued reference to Formulae IIA, IIB and IIC, R₄ independently is selected from H, and optionally substituted lower aliphatic, such as optionally substituted lower alkyl, acetyl and propanyl. In exemplary embodiments, R₄ is H, and in other embodiments R₄ is lower alkyl, such as methyl.

With continued reference to Formulas IIA, IIB and IIC, R₅ independently is selected from H, and optionally substituted lower aliphatic, such as optionally substituted lower alkyl. In exemplary embodiments, R₅ is H, and in other embodiments R₅ is lower alkyl, such as methyl or ethyl.

Particular examples of compounds have Formulae IIA, IIB and IIC, wherein X, R₁, R₂, R₃, R₄ and R₅ independently are as follows:
X═NH, NHCH₃, S; R₁═CH(CH₃)CH₃, R₂═R₃═H, R₄═H, CH₃, COCH₃, R₅═H, CH₃
X═O, NH, S; R₁═CH₃, R₂═H, CH₃, R₃═H, R₄═H, CH₃, COCH₃, R₅═H, CH₃
X═O, NH, S; R₁═CH₂CH₃, R₂═H, CH₃, R₃═H, R₄═H, CH₃, COCH₃, R₅═H, CH₃
X═O, NH, S; R₁═CH₂CH₂CH₃, R₂═H, CH₃, R₃═H, R₄═H, CH₃, COCH₃, R₅═H, CH₃
X═O, NH, S; R₁═CH₂NH₂, R₂═H, CH₃, R₃═H, R₄═H, CH₃, COCH₃, R₅═H, CH₃
X═O, NH, S; R₁═CH₂CH₂NH₂, R₂═H, CH₃, R₃═H, R₄═H, CH₃, COCH₃, R₅═H, CH₃
X═O, NH, S; R₁═CH(CH₃)NH₂, R₂═H, CH₃, R₃═H, R₄═H, CH₃, COCH₃, R₅═H, CH₃
X═O, NII, S; R₁═CII(NII₂)CII₂NII₂, R₂═II, CII₃, R₃═II, R₄═II, CII₃, COCH₃, R₅═II, CH₃
X═O, NH, S; R₁═CH₂OH, R₂═H, CH₃, R₃═H, R₄═H, CH₃, COCH₃, R₅═H, CH₃
X═O, NH, S; R₁═CH₂CH₂OH, R₂═H, CH₃, R₃═H, R₄═H, CH₃, COCH₃, R₅═H, CH₃
X═O, NH, S; R₁═CH(CH₃)OH, R₂═H, CH₃, R₃═H, R₄═H, CH₃, COCH₃, R₅═H, CH₃
X═O, NH, S; R₁═CH(OH)CH₂OH, R₂═H, CH₃, R₃═H, R₄═H, CH₃, COCH₃, R₅═H, CH₃
X═O, NH, S; R₁═CHO, R₂═H, CH₃, R₃═H, R₄═H, CH₃, COCH₃, R₅═H, CH₃
X═O, NH, S; R₁═CH₂CHO, R₂═H, CH₃, R₃═H, R₄═H, CH₃, COCH₃, R₅═H, CH₃
X═O, NH, S; R₁═CH₂COOH, R₂═H, CH₃, R₃═H, R₄═H, CH₃, COCH₃, R₅═H, CH₃
X═O, NH, S; R₁═COOH, R₂═H, CH₃, R₃═H, R₄═H, CH₃, COCH₃, R₅═H, CH₃
X═O, NH, S; R₁═NH₂, R₂═H, CH₃, R₃═H, R₄═H, CH₃, COCH₃, R₅═H, CH₃
X═O, NH, S; R₁═OH, R₂═H, CH₃, R₃═H, R₄═H, CH₃, COCH₃, R₅═H, CH₃
X═N, R₁═O, R₂═OH, R₃═H, R₄═H, CH₃, COCH₃, R₅═H, CH₃

Still additional structural modifications of the GAF molecule specifically contemplated for use in the methods and compositions disclosed herein include, without limitation, those of Formula III, used to inhibit or arrest weed seed germination:

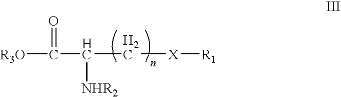

wherein the variable group X typically is selected from O, S and optionally substituted amino, and R₁ independently is selected from H, optionally substituted lower aliphatic, such as lower alkyl, optionally substituted amino, alkoxy, hydroxy, —NHCOOH, hydroxy alkyl, alkyl amino and the like, and wherein n=1, 2, 3, 4, or 5.

With continued reference to Formula III, R₂ independently is selected from H, and optionally substituted lower aliphatic, such as optionally substituted lower alkyl, acetyl and propanyl. In exemplary embodiments, R₂ is H, and in other embodiments R₂ is lower alkyl, such as methyl.

With continued reference to Formula III, R₃ independently is selected from H, and optionally substituted lower aliphatic, such as optionally substituted lower alkyl. In exemplary embodiments, R₃ is H, and in other embodiments R₃ is lower alkyl, such as methyl or ethyl.

In particular examples, the structural modifications of the GAF molecule used in the present methods and compositions include those of Formula III wherein n=1 and R₁, R₂, R₃ and X are:
X═O, NH, S; R₁═CH₂CH₂CH₂NH₂, R₂═H, CH₃, COCH₃, R₃═H, CH₃
X═O, NH, S; R₁═CH₂CH(NH₂)CH₂NH₂, R₂═H, CH₃, COCH₃, R₃═H, CH₃
X═O, NH, S; R₁═CH₂CH(OH)CH₂OH, R₂═H, CH₃, COCH₃, R₃═H, CH₃
X═O, NH, S; R₁═CH₂CHO, R₂═H, CH₃, COCH₃, R₃═H, CH₃
X═O, NH, S; R₁═CH₂CH₂CHO, R₂═H, CH₃, COCH₃, R₃═H, CH₃
X═O, NH; R₁═CH₂COOH, R₂═H, CH₃, COCH₃, R₃═H, CH₃
X═O, NH, S; R₁═CH₂CH₂COOH, R₂═H, CH₃, COCH₃, R₃═H, CH₃
X═O; R₁═NH₂, R₂═H, CH₃, COCH₃, R₃═H, CH₃
   and wherein n=2, R₁, R₂, R₃ and X are:
X═NH; R₁═OH, OCH₃, R₂═H, CH₃, COCH₃, R₃═H, CH₃
X═O; R₁═H, NH₂, R₂═H, CH₃, COCH₃, R₃═H, CH₃
X═O, NH; R₁═CH₃, R₂═H, CH₃, COCH₃, R₃═H, CH₃
X═O, NH, S; R₁═CH₂CH₃, R₂═H, CH₃, COCH₃, R₃═H, CH₃
X═O, NH, S; R₁═CH₂CH₂CH₃, R₂═H, CH₃, COCH₃, R₃═H, CH₃
X═O, NH, S; R₁═CH₂CH₂CH₂CH₃, R₂═H, CH₃, COCH₃, R₃═H, CH₃
X═O, NH, S; R₁═CH₂CH(CH₃)CH₃, R₂═H, CH₃, COCH₃, R₃═H, CH₃
X═O, NH, S; R₁═CH₂NH₂, R₂═H, CH₃, COCH₃, R₃═H, CH₃
X═O, NH; S; R₁═CH₂CH₂NH₂, R₂═H, CH₃, COCH₃, R₃═H, CH₃
X═O, NH, S; R₁═CH₂CH₂CH₂NH₂, R₂═H, CH₃, COCH₃, R₃═H, CH₃
X═O, NH, S; R₁═CH₂CH(CH₃)NH₂, R₂═H, CH₃, COCH₃, R₃═H, CH₃
X═O, NH, S; R₁═CH₂CH(NH₂)CH₂NH₂, R₂═H, CH₃, COCH₃, R₃═H, CH₃
X═O, NH, S; R₁═CH₂OH, R₂═H, CH₃, COCH₃, R₃═H, CH₃
X═O, NH, S; R₁═CH₂CH₂OH, R₂═H, CH₃, COCH₃, R₃═H, CH₃
X═O, NH, S; R₁═CH₂CH₂CH₂OH, R₂═H, CH₃, COCH₃, R₃═H, CH₃
X═O, NH, S; R₁═CH₂CH(CH₃)OH, R₂═H, CH₃, COCH₃, R₃═H, CH₃
X═O, NH, S; R₁═CH₂CH(OH)CH₂OH, R₂═H, CH₃, COCH₃, R₃═H, CH₃
X═O, NH, S; R₁═CH₂CHO, R₂═H, CH₃, COCH₃, R₃═H, CH₃
X═O, NH, S; R₁═CH₂CH₂CHO, R₂═H, CH₃, COCH₃, R₃═H, CH₃
X═O, NH, S; R₁═CH₂COOH, R₂═H, CH₃, COCH₃, R₃═H, CH₃
X═O, NH, S; R₁═CH₂CH₂COOH, R₂═H, CH₃, COCH₃, R₃═H, CH₃
X═O; R₁═NHCOCH₃, R₂═H, CH₃, COCH₃, R₃═H, CH₃
X═O; R₁═NH(COCH₃)₂, R₂═H, CH₃, COCH₃, R₃═H, CH₃
X═O; R₁═NHCOOH, R₂═H, CH₃, COCH₃, R₃═H, CH₃
X═O; R₁═NHCOOCH₃, R₂═H, CH₃, COCH₃, R₃═H, CH₃
X═O; R₁═NHCHO, R₂═H, CH₃, COCH₃, R₃═H, CH₃

Still other structural modifications of the GAF molecule for use in the present methods and compositions include those of Formula IV:

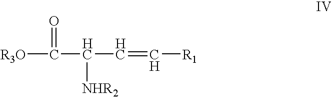

wherein
R₁═NHOH, R₂═H, CH₃, COCH₃, R₃═H, CH₃, CH₃CH₂
R₁═NHOCH₃, R₂═H, CH₃, COCH₃, R₃═H, CH₃, CH₃CH₂
R₁═OCH₂CH₂NH₂, R₂═H, CH₃, COCH₃, R₃═H, CH₃, CH₃CH₂
R₁═OCH₃, R₂═H, CH₃, COCH₃, R₃═H, CH, CH₃CH₂₃
R₁═ONH₂, R₂═H, CH₃, COCH₃, R₃═H, CH₃, CH₃CH₂
R₁═CH₂PO₃²⁻, R₂═H, CH₃, COCH₃, R₃═H, CH₃, CH₃CH₂
R₁═CH₂CH₂CO₂H, R₂═H, CH₃, COCH₃, R₃═H, CH₃, CH₃CH₂
R₁═CHCH₂, R₂═H, CH₃, COCH₃, R₃═H, CH₃, CH₃CH₂
R₁═CH₂NHCONH₂, R₂═H, CH₃, COCH₃, R₃═H, CH₃, CH₃CH₂

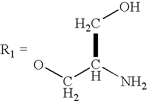

### R₂═H, CH₃, COCH₃, R₃═H, CH₃, CH₃CH₂

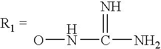

### R₂═H, CH₃, COCH₃, R₃═H, CH₃, CH₃CH₂

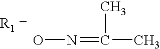

### R₂═H, CH₃, COCH₃, R₃═H, CH₃, CH₃CH₂

Additional compounds according to Formula IV suitable for use in the present methods and compositions include alkyloxy and aryloxy vinylglycines, including those wherein

R₁═OCH₂CH(CH₃)CH₃, R₂═H, CH₃, COCH₃, R₃═H, CH₃, CH₃CH₂

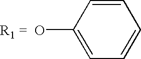

### R₂═H, CH₃, COCH₃, R₃═H, CH₃, CH₃CH₂

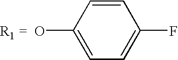

### R₂═H, CH₃, COCH₃, R₃═H, CH₃, CH₃CH₂

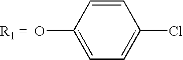

### R₂═H, CH₃, COCH₃, R₃═H, CH₃, CH₃CH₂

Still other compounds of Formula IV are thiovinylglycines, wherein R₁ is a substituted thiol moiety, such as a lower alkyl substituted thiol moiety, for example wherein R₁═S—CH₃. R₂═H, CH₃, COCH₃, R₃═H, CH₃, CH₃CH₂.

In one embodiment, suitable structural modifications of the GAF molecule for use in the present methods and compositions include those of the formulas

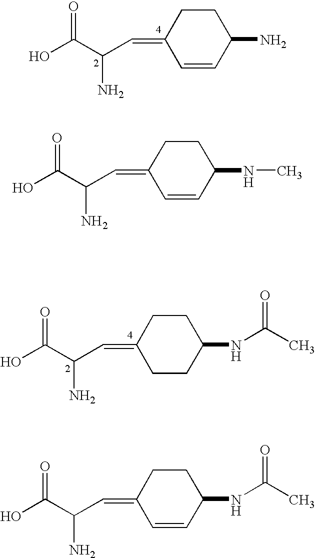

Additional examples of structural modifications of the GAF molecule include, without limitation, β,γ-disubstituted vinylic amino acids of the general formula V

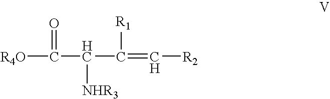

wherein the substituents R₁ and R₂ independently are selected from optionally substituted lower aliphatic, such as lower alkyl, and halo, in particular chloro and bromo, R₃ is selected from H, lower alkyl, acetyl and propanyl, and R₄ is selected from H and lower alkyl.

R₁═CH₂OH; R₂═CH₃, R₃═H, CH₃, COCH₃, R₄═H, CH₃

R₁═CHO; R₂═CH₃, R₃═H, CH₃, COCH₃, R₄═H, CH₃

R₁═Cl; R₂═CO₂H, R₃═H, CH₃, COCH₃, R₄═H, CH₃:

Other organic acids, for example those that share with vinylglycines the ability to act as inhibitors of pyridoxal-phosphate dependent enzyme reactions, including reactions catalyzed by aminotransferases and the reaction catalyzed by ACC synthase in ethylene biosynthesis, also may be useful in the present methods and compositions either alone or in combination with GAF or other molecules structurally related to GAF. Examples of such organic acids include by way of example:
H₂N—O—CH₂—COOH: 2-Aminooxyacetic acid
OHC—NH—O—CH₂—COOH: 2-Formylaminooxyacetic acid
H₂N—O—CH₂—CH₂—COOH: 3-Aminooxypropanoic acid
OHC—NH—O—CH₂—CH₂—COOH: 2-Formylaminooxypropanoic acid
H₂N—O—CH₂—CH₂—CH₂—COOH: 3-Aminooxybutanoic acid
OHC—NH—O—CH₂—CH₂—CH₂—COOH: 3-Formylaminooxybutanoic acid

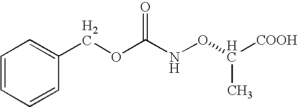

N-benzyloxycarbonyl-L-2-aminooxypropanoic acid

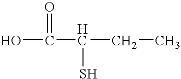

2-mercaptobutanoic acid
and cyclic amino acids of the formula VI:

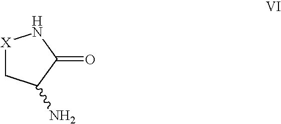

for example those wherein
X═O: Cycloserine
X═S: (R/S)-4-Amino-3-isothiazolidinone
X═N: Azacycloserine {(R/S)-4-Amino-3-Pyrazolidinone}

Additional structural modifications of the GAF molecule will be apparent to those of ordinary skill in the art upon consideration of the formulas above and further in view of the entirety of the present disclosure.

### VI. Production of Pseudomonas Extracts

GAF was originally identified in particular isolates of certain species of the rhizobacteria Pseudomonas. In general, Pseudomonas isolates are inoculated into Pseudomonas Minimal Salts Medium and incubated with gentle agitation for several days prior to harvest. The cultures are then centrifuged, and the supernatant is filtered.

Measured volumes of the culture filtrate are dried, and the dry solids recovered from the culture filtrate are extracted with ethanol or other solvents. The dry solids are then redissolved in a solvent appropriate to the planned experiment prior to use.

Additional description regarding production of extracts from Pseudomonas isolates can be found in PCT/US03/38653 (published as WO 04/052097; incorporated herein by reference in its entirety).

As described herein, bacteria other than Pseudomonas are capable of producing GAF and having GAF-type activity. Methods of culturing and producing extracts from such bacteria are well known to those of skill in the art.

### VII. Seed Germination Assays

Any seed germination assay can be used to measure GAF activity. One such assay is the Standard GAF Bioassay System described herein. In the Standard GAF Bioassay System, aliquots of each test solution are distributed in different wells of a sterile multi-well culture plate. Surface-sterilized seeds are transferred aseptically to each well and submerged in the test solution. The culture plate is then sealed and incubated in a germinator for 7 days prior to scoring seed germination.

Seed germination also can be determined using a germination assay on filter paper. Surface sterilized seeds are vacuum dried and then sprinkled on glass-microfiber filter discs wetted with sterilized water and placed in a Petri dish. The Petri dishes are sealed with Parafilm® packaged in aluminum foil and put in a growth chamber for a preconditioning period.

After the preconditioning period, Petri dishes are opened and individual seeds are placed on individual glass-microfiber discs treated with sterile water or test solution. All Petri dishes are then resealed with Parafilm® and returned to the growth chamber. Several days later, the seed germination rate is determined under a stereoscopic microscope by counting the number of seeds with and without an emerged radical.

Any other seed germination assay known in the art can be used to screen for GAF activity assuming that a seed sensitive to the action of GAF (e.g. Poa annua) is used as the test material. For example assays outlined in the Ecological Effects Test Guidelines: OPPTS 850.4225 Seedling Emergence Tier II, Guidelines developed by the Office of Prevention, Pesticides and Toxic Substances, United States Environmental Protection Agency (available on-line at gopher.epa.gov)

### VIII. GAF Formulations and Formulations Involving Structurally Related Compounds

GAF (2-amino-4-formylaminooxybut-3-enoic acid) and the structurally related compounds of the present disclosure can be combined with appropriate solvents or surfactants to form a product called a formulation. Formulations enable the uniform distribution of a relatively small amount of GAF, or structurally related compounds of similar biological activity, over a comparatively large area. In addition to providing the user with a form of GAF that is easy to handle, formulating GAF can enhance its phytotoxicity, improve its shelf-life, and protect it from adverse environmental conditions while in storage or transit.

The primary kinds of herbicide formulations are: solutions, soluble powders, emulsifiable concentrates, wettable powders, liquid flowables, dry flowables, water-dispersible granules, granules, and pellets. Formulations vary according to the solubility of the herbicide active ingredient in water, oil and organic solvents, and the manner the formulation is applied (i.e., dispersed in a carrier, such as water, or applied as a dry formulation).

Solution formulations are designed for those active ingredients that dissolve readily in water. The formulation is a liquid and consists of the active ingredient and additives. Generally, when herbicides formulated as solutions are mixed with water, the active ingredient will not settle out of solution or separate. Suitable liquid carriers, such as solvents, may be organic or inorganic. Water is one example of an inorganic liquid carrier. Organic liquid carriers include vegetable oils and epoxidized vegetable oils, such as rape seed oil, castor oil, coconut oil, soybean oil and epoxidized rape seed oil, epoxidized castor oil, epoxidized coconut oil, epoxidized soybean oil, and other essential oils. Other organic liquid carriers include silicone oils, aromatic hydrocarbons, and partially hydrogenated aromatic hydrocarbons, such as alkylbenzenes containing 8 to 12 carbon atoms, including xylene mixtures, alkylated naphthalenes, or tetrahydronaphthalene. Aliphatic or cycloaliphatic hydrocarbons, such as paraffins or cyclohexane, and alcohols, such as ethanol, propanol or butanol, also are suitable organic carriers. Gums, resins, and rosins used in forest products applications and naval stores (and their derivatives) also may be used. Additionally, glycols, including ethers and esters, such as propylene glycol, dipropylene glycol ether, diethylene glycol, 2-methoxyethanol, and 2-ethoxyethanol, and ketones, such as cyclohexanone, isophorone, and diacetone alcohol may be used. Strongly polar organic solvents include N-methylpyrrolid-2-one, dimethyl sulfoxide, and N,N-dimethylformamide.

Typical liquid diluents and solvents are described in Marsden, Solvents Guide, 2nd Ed., Interscience, NY, 1950. Solubility under 0.1% is preferred for suspension concentrates; solution concentrates are preferably stable against phase separation at 0° C. McCutcheon's Detergents and Emulsifiers Annual, Allured Publ. Corp., Ridgewood, N.J., as well as Sisely and Wood, Encyclopedia of Surface Active Agents, Chemical Publ., Co., Inc., NY 1964, list surfactants and recommended uses.

Soluble powder formulations are similar to solutions in that, when mixed with water, they dissolve readily and form a true solution. Soluble powder formulations are dry and include the active ingredient and additives. When thoroughly mixed, no further agitation is necessary to keep the active ingredient dissolved in solution.

Emulsifiable concentrate formulations are liquids that contain the active ingredient, one or more solvents, and an emulsifier that allows mixing with water. Formulations of this type are highly concentrated, relatively inexpensive per pound of active ingredient, and easy to handle, transport, and store. In addition, they require little agitation (will not settle out or separate) and are not abrasive to machinery or spraying equipment. Formulations of this type may, however, have greater phytotoxicity than other formulations, and they are subject to over- or under-dosing through mixing or calibration errors. In addition, these types of formulations are more easily absorbed through skin of humans or animals, and contain solvents that may cause deterioration of rubber or plastic hoses and pump parts.

Wettable powders are dry, finely ground formulations in which the active ingredient is combined with a finely ground carrier (usually mineral clay), along with other ingredients to enhance the ability of the powder to suspend in water. Generally, the powder is mixed with water for application. Wettable powders are some of the most widely used herbicide formulations and offer low cost and ease of storage, transport, and handling; lower phytotoxicity potential than emulsifiable concentrates and other liquid formulations; and less skin and eye absorption hazard than emulsifiable concentrates and other liquid formulations. Some disadvantages are that they require constant and thorough agitation in the spray tank, are abrasive to pumps and nozzles (causing premature wear), may produce visible residues on plant and soil surfaces, and can create an inhalation hazard to the applicator while handling (pouring and mixing) the concentrated powder. Typical solid diluents are described in Watkins et al., Handbook of Insecticide Dust Diluents and Carriers, 2nd Ed., Dorland Books, Caldwell, N.J. The more absorptive diluents are preferred for wettable powders and the denser ones for dusts.

Liquid flowable formulations are made up of finely ground active ingredient suspended in a liquid. Flowables generally are mixed with water for application, are easily handled and applied, and seldom clog nozzles. Some of their disadvantages are that they may leave a visible residue on plant and soil surfaces, and typically require constant and thorough agitation to remain in suspension.

Dry flowable and water-dispersible granule formulations are much like wettable powders except that the active ingredient is formulated on a large particle (granule) instead of onto a ground powder. This type of formulation offers essentially the same advantages and disadvantages as wettable powder formulations. However, these formulations generally are more easily mixed and measured than wettable powders. Because they create less dust when handling, they cause less inhalation hazard to the applicator during pouring and mixing.

Granules and pellets are used for soil-applied herbicides. In a granule or pellet formulation, the active ingredient is formulated onto large particles (granules or pellets). The primary advantages of this type of formulation are that the formulation is ready to use with simple application equipment (seeders or spreaders), and the drift potential is low because the particles are large and settle quickly. The disadvantages of these formulations are that they do not adhere to foliage (not intended for foliar applications), and may require mixing into the soil in order to achieve adequate herbicidal activity.

Granulated materials of inorganic or organic nature may be used in formulating granules and pellets, such as dolomite or pulverized plant residues. Suitable porous granulated adsorptive carriers include pumice, broken brick, sepiolite, and bentonite. Additionally, nonsorbent carriers, such as sand, may be used. Some solid carriers are biodegradable polymers, including biodegradable polymers that are digestible or degrade inside an animal's body over time.

The methods of making such formulations are well known. Solutions are prepared by simply mixing the ingredients. Fine, solid compositions are made by blending and, usually, grinding, as in a hammer or fluid energy mill. Suspensions are prepared by wet-milling (see, for example, U.S. Pat. No. 3,060,084). Granules and pellets may be made by spraying the active material upon preformed granular carriers or by agglomeration techniques. See J. E. Browning, “Agglomeration”, Chemical Engineering, Dec. 4, 1967, p 147, and Perry's Chemical Engineer's Handbook, 4th Ed., McGraw-Hill, NY, 1963, pp. 8-59. For further information regarding the art of formulation, see, for example: U.S. Pat. No. 3,235,361, U.S. Pat. No. 3,309,192, U.S. Pat. No. 2,891,855, Klingman, Weed Control as a Science, John Wiley & Sons, Inc., New York, 1961 pp. 81-96, and Fryer and Evans, Weed Control Handbook, 5th Edn. Blackwell Scientific Publications, Oxford, 1968, pp. 101-103.

In selecting a formulation for an herbicide, the following considerations may be weighed: 1) how the formulation will affect the phytotoxicity of undesirable plants and/or desirable plant, 2) how the formulation will influence the compatibility of other crop protection chemicals, 3) what application machinery are available and most suited for the job, 4) how the formulation will affect the life of the application equipment, 5) whether the application equipment is designed for applying a particular formulation, and 6) concerns about safety for the applicator and other people.

The concentration of a compound, such as GAF, which serves as an active ingredient, may vary according to particular compositions and applications. In some embodiments of the disclosure, the percentage by weight of the active ingredient will be from about 0.1% to about 90%, for example to give final GAF concentrations equivalent to, or greater than, that in Pseudomonas fluorescens WH6 culture filtrate. A suitable amount for a particular application may be determined using bioassays for the particular pest intended to be controlled. Higher concentrations are usually employed for commercial purposes or products during manufacture, shipment, or storage; such embodiments have concentrations at least about 10%, or from about 25% to about 90% by weight. Prior to use, a highly concentrated formulation may be diluted to a concentration appropriate for the intended use, such as from about 0.1% to 10%, or from about 1% to 5%.

### IX. GAF-Producing Bacterial Strains, Bacterial Strains that Produce Other Vinylglycines, and Formulations Thereof

Based on the disclosure herein, it can now be appreciated that any Pseudomonas bacterium having certain characteristic biosynthetic gene sequences (characterized by for instance, the formyl-transferase described in Example 5) is capable of producing GAF (specifically, 2-amino-4-formylaminooxy-but-3-enoic acid) or other vinylglycine molecules which inhibit weed germination. As further described in Examples 5-7, it can also be appreciated that any bacterium of any species that produces GAF or related molecules can be utilized to inhibit weed germination.

As will be understood by those with skill in the art, with reference to this disclosure, in order for bacterial strains which produce GAF (2-amino-4-formylaminooxy-but-3-enoic acid) or other vinylglycine molecules which inhibit weed germination to be grown, cultured, or used in accordance with the embodiments of the present invention, these bacterial strains will be grown in a medium suitable to produce a biocontrol composition or formulation. The term “suitable medium” or “acceptable medium” is meant to include any liquid, semi-liquid, or solid substrate which allows GAF-producing bacterial strains or bacterial strains that produce other vinylglycines to grow, or to remain viable, or both grow and remain viable, for example during storage. Furthermore, these bacterial strains may be formulated as indicated below prior to use. Such formulations are also considered suitable or acceptable media in the context of the present invention. Preferably, the formulation permits an effective amount of one or more GAF-producing bacterial strains to remain viable prior to, and after, being applied to a crop. More preferably, the medium, formulation, or both medium and formulation permits GAF-producing bacterial strains or bacterial strains that produce other vinylglycines that inhibit weed germination to remain viable for about 1 to 3 months following application of the bacteria to the soil.

The present invention also contemplates growing GAF-producing bacterial strains or bacterial strains that produce other types of vinylglycines that inhibit weed germination in various types of media, including, but not limited to, Pseudomonas Minimal Salts (PMS) medium with or without additional supplements, 925 minimal medium, nutrient broth, M9 medium and REC medium. The present invention also contemplates formulations of the bacteria in pesta, peat prills, vermiculite, clay, starches, wheat straw (see for example Connick et al., 1991; Fravel, Connick and Lewis, 1998. Formulation of microorganisms to control plant diseases. p. 187-202 In: H. D. Burges (Ed.), Formulation of Microbial Biopesticides, Kluwer Academic Publishers, Dordrecht, The Netherlands.; Quimby et al., Biocontrol Science and Technology 9:5-8, 1999; U.S. Pat. Nos. 5,074,902; 5,358,863; and International Publication WO 98/05213), or any combination or variant thereof, provided that the formulation allows the bacterial strain that produces GAF molecule or other vinylglycine molecule to remain viable. The biocontrol agent may also be applied to the surface of the seed in a suitable formulation or composition as would be known to one of skill in the art. Furthermore, it is contemplated that the GAF-producing bacterial strains or bacterial strains that produce other vinylglycines that inhibit weed germination may be applied in a suitable formulation before, during or after seeding a crop.

### X. Inactive Ingredients in Formulations

In some embodiments of the disclosure, inactive ingredients (that is, adjuvants) are added to herbicide formulations to improve the performance of the herbicide. For example, in one embodiment of the disclosure, GAF molecule(s) is formulated with a surfactant. A surfactant (surface active agent) is a type of adjuvant designed to improve the dispersing/emulsifying, absorbing, spreading, sticking and/or pest-penetrating properties of the spray mixture. Surfactants can be divided into the following five groupings: 1) non-ionic surfactants, 2) crop oil concentrates, 3) nitrogen-surfactant blends, 4) esterified seed oils, and 5) organo-silicones.

Suitable surfactants may be nonionic, cationic, or anionic, depending on the nature of the compound used as an active ingredient. Surfactants may be mixed together in some embodiments of the disclosure. Nonionic surfactants include polyglycol ether derivatives of aliphatic or cycloaliphatic alcohols, saturated or unsaturated fatty acids and alkylphenols. Fatty acid esters of polyoxyethylene sorbitan, such as polyoxyethylene sorbitan trioleate, also are suitable nonionic surfactants. Other suitable nonionic surfactants include water-soluble polyadducts of polyethylene oxide with polypropylene glycol, ethylenediaminopolypropylene glycol and alkylpolypropylene glycol. Particular nonionic surfactants include nonylphenol polyethoxyethanols, polyethoxylated castor oil, polyadducts of polypropylene and polyethylene oxide, tributylphenol polyethoxylate, polyethylene glycol and octylphenol polyethoxylate. Cationic surfactants include quaternary ammonium salts carrying, as N-substituents, an 8 to 22 carbon straight or branched chain alkyl radical.

The quaternary ammonium salts carrying may include additional substituents, such as unsubstituted or halogenated lower alkyl, benzyl, or hydroxy-lower alkyl radicals. Some such salts exist in the form of halides, methyl sulfates, and ethyl sulfates. Particular salts include stearyldimethylammonium chloride and benzyl bis(2-chloroethyl)ethylammonium bromide. Suitable anionic surfactants may be water-soluble soaps as well as water-soluble synthetic surface-active compounds. Suitable soaps include alkali metal salts, alkaline earth metal salts, and unsubstituted or substituted ammonium salts of higher fatty acids. Particular soaps include the sodium or potassium salts of oleic or stearic acid, or of natural fatty acid mixtures. Synthetic anionic surfactants include fatty sulfonates, fatty sulfates, sulfonated benzimidazole derivatives, and alkylarylsulfonates. Particular synthetic anionic surfactants include the sodium or calcium salt of ligninsulfonic acid, of dodecyl sulfate, or of a mixture of fatty alcohol sulfates obtained from natural fatty acids. Additional examples include alkylarylsulfonates, such as sodium or calcium salts of dodecylbenzenesulfonic acid, or dibutylnaphthalenesulfonic acid. Corresponding phosphates for such anionic surfactants are also suitable.

Other adjuvants include carriers and additives, for example, wetting agents, such as anionic, cationic, nonionic, and amphoteric surfactants, buffers, stabilizers, preservatives, antioxidants, extenders, solvents, emulsifiers, invert emulsifiers, spreaders, stickers, penetrants, foaming agents, anti-foaming agents, thickeners, safeners, compatibility agents, crop oil concentrates, viscosity regulators, binders, tackers, drift control agents, or other chemical agents, such as fertilizers, antibiotics, fungicides, nematicides, or pesticides. Such carriers and additives may be used in solid, liquid, gas, or gel form, depending on the embodiment and its intended application.

Additionally, the composition may include plural herbicidal compounds. Such a composition includes GAF as described herein and a second herbicidal compound.

### XI. Methods of Use

GAF and the structurally related compounds disclosed herein (i.e. the natural product, 2-amino-4-formylaminooxy-but-3-enoic acid, and structural modifications and derivatives of this compound as indicated herein) may be used to control grassy weeds and cultivated grasses, for example, but not limited to, annual bluegrass, downy brome, jointed goatgrass, roughstalk bluegrass, and rattail fescue, perennial ryegrass, and tall fescue, in addition to both domestic and wild oats. GAF and the structurally related compounds disclosed herein may also be used to control other types of weeds if these weeds propagate by seed that is germinating among established plants or among other seeds that are insensitive to GAF. In one embodiment of the disclosure, GAF or compounds that are structurally related to GAF are used in grass seed production systems or other cropping systems. In other embodiments of the disclosure, GAF or compounds that are structurally related to GAF are used in natural settings, horticultural landscapes, or any setting where grassy weeds are problematic. One specific, non-limiting example of a method of use of GAF and structurally related compounds is to control grassy weeds in natural settings, such as wilderness areas, lands under control of the Bureau of Land Management, or in ecologically sensitive areas. Another specific, non limiting example of a method of use is to control grassy weeds along roadsides. In any particular embodiment of the disclosure, GAF or compounds that are structurally related to GAF are applied in an herbicidally effective amount. That amount may depend on a variety of factors, including (but not limited to) the area to be treated, the seed or growth medium to be treated, or the mechanism of application.

In one embodiment of the disclosure, GAF or compounds that are structurally related to GAF are applied by direct application to the growth medium, for example soil. Soil-applied herbicides may be used at various times, including several weeks before crop planting (early preplant), just prior to crop planting (preplant or preplant-incorporated), immediately after crop planting but before weed emergence (preemergence), or after crop and generally weed emergence (postemergence), and directed onto the soil or weeds while preventing contact with the crop (post-directed). Activity of soil-applied herbicides is influenced by soil texture, organic matter content, pH, water status, and tillage. Herbicide uptake may occur via roots, seeds, shoots, and vegetative propagules.

In one embodiment of the disclosure, GAF or compounds that are structurally related to GAF are applied as early preplant herbicides. Early preplant herbicides are applied to the soil weeks in advance of crop planting, so herbicides applied in this manner generally have sufficient residual activity in soils to extend through the first few weeks after emergence.

In another embodiment of the disclosure, GAF or compounds that are structurally related to GAF are applied as preplant-incorporated herbicides. Generally, preplant-incorporated herbicides are physically mixed into the soil prior to crop planting. Incorporation usually is performed with some type of secondary tillage implement that distributes the herbicide evenly in the top two to three inches of the soil.

In another embodiment of the disclosure, GAF or compounds that are structurally related to GAF are applied as preemergence herbicides. Preemergence herbicides generally are applied to the soil prior to crop and weed emergence. In annual crops, preemergence herbicides are usually applied immediately after the crop is planted. Herbicides applied to the soil in perennial crops, turf grass, pastures, or non-cropland areas before weed seedlings emerge are also considered preemergence herbicides. Rainfall or irrigation moves preemergence herbicides into the top few inches of soil for optimum activity.

In other embodiments of the disclosure, GAF or compounds that are structurally related to GAF are applied as foliar-applied herbicides to control developing weed seeds. The effectiveness of foliar-applied herbicides is determined by the rate and timing of application, target weed species, use of spray adjuvants, application equipment, and environmental conditions. Foliar penetration of herbicides is greatest in non-stressed plants under conditions of moderate temperature and high relative humidity. A rain-free period following postemergence herbicide application is helpful to allow a toxic dosage of the herbicide to be absorbed by the target plants.

In other embodiments of the disclosure, GAF or compounds that are structurally related to GAF are applied as a seed-cleaning adjuvant in seed-cleaning processes as a supplement or alternative to physical removal of target weed seeds.

In some embodiments of the disclosure, GAF or compounds that are structurally related to GAF are applied once, while alternative embodiments of the disclosure employ plural applications of GAF or structurally related compounds. In particular embodiments of the disclosure, GAF or compounds that are structurally related to GAF are applied on a weekly, monthly, quarterly, or annual basis. In any particular embodiment of the disclosure, the frequency of application may be regular or irregular, and the time elapsed between successive applications may be the same or different. For example, and without limitation, GAF or structurally related compounds may be applied every other week; every other month; twice a month; every three months; every six months; every nine months; or annually. The frequency and number of applications of GAF or structurally related compounds may depend on a variety of factors, including (but not limited to) the area to be treated, the seed variety to be treated, environmental conditions, and the method of application.

In certain embodiments of the disclosure, GAF or compounds that are structurally related to GAF are applied in an area-wide manner, such as in protection of agricultural crops, for example grass or wheat crops. In addition to agricultural applications, area-wide applications may include silvicultural, horticultural, or other forms of environmental grassy weed management and control. In such embodiments of the disclosure, GAF or structurally related compounds may be applied to the soil, such as drenching a particular locus with a liquid formulation or applying the active ingredient in solid form to a locus.

Certain embodiments use GAF or compounds that are structurally related to GAF for grassy weed control in seed production and storage. For example, GAF or structurally related compounds may be applied to non-targeted crop seeds to arrest germination of grassy weeds. In some examples, GAF or structurally related compounds are applied to the seed by soaking, coating, or dressing seeds prior to sowing. In another example, GAF or structurally related compounds are applied to a physical substrate that serves as a germination barrier as in seed strips for landscape applications.

GAF or compounds that are structurally related to GAF also may be applied to the soil where the seeds will be planted, such as in-furrow or in-field application. In such applications, GAF compounds may be applied to provide a certain concentration of the compound in the environment at a particular locus. That certain concentration may be measured, established, or determined according to the needs of the user. For example, when applying GAF or structurally related compounds to crops, the rate of application may depend on the nature of soil, the type of application (e.g., spraying crop foliage, burial in soil), the crop plant to be protected, the weed species to be controlled, the prevailing climatic conditions, the growing season, and other factors.

As another example, when applying GAF or compounds that are structurally related to GAF to stored or transported agricultural products, the rate of application may depend on the localized environment (e.g., storage within a warehouse, storage under a covered shelter, transport within a trailer), expected duration of storage, product to be protected, the weed species to be controlled, economic considerations, and other factors. In certain embodiments, the rates of concentration are in the range from about 0.01 to about 1000 ppm (parts-per-million), such as from about 0.1 to about 500 ppm, of active ingredient. In area-wide applications, rates of application per hectare may be from about 0.5 g/ha to 2000 g/ha, such as particularly from about 10 to 1000 g/ha, or from about 20 to 600 g/ha.

In some embodiments of the disclosure, GAF-producing bacterial strains or bacterial strains that produce other vinylglycines may themselves be used as biocontrol agents to control weed germination in areas containing targeted weed seeds.

As will be understood by those with skill in the art, with reference to this disclosure, in order for bacterial strains which produce GAF (2-amino-4-formylaminooxy-but-3-enoic acid) or for bacterial strains that produce other vinylglycine molecules to be grown, cultured, or used in accordance with the embodiments of the present invention, these bacterial strains will be applied as an aqueous suspensions or on an inert carrier. The solution or carrier preferably contains between about 10³ and 10⁸ cells per gram (or ml for a solution) but any larger or smaller numbers of the cells per gram or ml can be used so long as suppression of germination and/or growth of the target weeds are achieved.

The present invention also contemplates that the bacterial strains will be provided for shipment to users in the form of a concentrate containing at least about 10⁶ cells per gram or to a greater concentration mixed with a preservative agent, the exact composition of which depends upon the method of bacterial cell preservation. The cells may be frozen or dried in various forms. Where the cells are frozen, glycerol or various sugars and fresh growth media of various compositions can be used as preservation agents. Amounts usually between 5 and 50% by volume of the glycerol or sugars can be used. Where the cells are dried, nutrient media of various compositions as would be known to one of skill in the art can be used for preservation. Generally, bacterial strains which produce GAF or other vinylglycine molecules which inhibit weed germination are grown to about 10⁹ cells per ml and may then be centrifuged or otherwise concentrated by removal of growth media. They can then be frozen or dried. The dried bacteria can be mixed with inorganic solid carriers such as clays, talc, inert organic material or the like which may then be applied directly to areas containing target weed seeds or mixed with water or other liquids or semi-liquids and sprayed on the target area.

All of these variations for storing, growing and applying bacterial strains which produce GAF or other vinylglycine molecules which inhibit weed germination will be obvious to those skilled in the art. Generally, a biologically pure culture or mixtures of pure cultures of bacterial strains which produce GAF or other vinylglycine molecules which inhibit weed germination can be used for the application to areas containing the target weed seeds. GAF-producing bacterial strains, and bacterial strains that produce other vinylglycine molecules may also be mixed with bacterial strains that produce other compounds that inhibit weed germination.

### XII. Kits

In some embodiments of the disclosure, GAF or compounds that are structurally related to GAF are embodied in an acceptable carrier and stored within a container capable of storing the composition for its shelf life. The container may be made of any suitable material such as plastic or other polymer, glass, metal, or the like. In some embodiments of the disclosure, printed instructions and/or a printed label indicating that the composition may be used to control grassy weeds are associated with this container. In certain examples, the instructions and/or label provides information regarding the use of the composition for herbicidal purposes, and is associated with the container by being adhered to the container, or accompanying the container in a package. In particular examples, the instructions specify the weeds intended to be controlled by the composition, the method and rate of application, dilution protocols, use precautions, and the like. Additionally, the container may include a feature or device for applying the composition to the seed population or locus to be treated. For example, if the article of manufacture includes a liquid composition, the feature or device may be a hand-operated, motorized, or pressurized pressure-driven sprayer.

In some embodiments, for large-scale applications, a GAF kit may include a drum, whereas for household kits for control of grassy weeds, GAF or compounds that are structurally related to GAF may be provided in a can or bottle. In other embodiments, GAF or compounds that are structurally related to are provided as small scale, highly purified material for experimental use in understanding plant developmental processes. In still other embodiments, GAF or compounds that are structurally related to GAF are provided as seed cleaning adjuvants.

In some embodiments, GAF-producing bacteria or bacterial strains that produce other vinylglycine molecules or mixtures of these with or without other bacterial strains that produce other types of agents with deleterious effects on weeds may be packaged as a kit consisting of an appropriate container containing the bacteria in various formulations to preserve shelf-life and ensure survival in the field together with instructions for the application and appropriate use of these bacterial preparations for weed control.

### Examples

### Example 1

### General Methods

### A. Origin and Storage of Isolates and Control Stains of Pseudomonas.

The origins of the various isolates of Deleterious Rhizosphere Bacteria (DRB) used in the studies reported in Example 5 are summarized in Table 6. As indicated there, these isolates were obtained by screening bacteria from the surface of roots of wheat, Triticale, and Poa species collected from a variety of locations in agricultural landscapes and roadsides throughout the Willamette Valley and the Coast Range of Western Oregon. Isolates obtained in this manner were selected for further study on the basis of their ability to inhibit root and shoot elongation in Annual Bluegrass (ABG, Poa annua) seedlings by more than 20% in tests where 7-day old seedlings were transferred to agar media inoculated with live bacteria, and seedling growth was measured after 10-days exposure to the test conditions. The bacterial isolates selected in this manner were transferred to the liquid Pseudomonas Minimal Salt Medium (PMS Medium) described below. The cultures were grown to an O.D.₅₈₀ of 1.0, and aliquots of the cultures were then transferred to cryovials and stored in 50% glycerol at −60° C. prior to use in this study.

### B. Growth of Bacterial Cultures

Pseudomonas isolates stored in cryovials (50% glycerol, −60° C.) were inoculated into Wheaton bottles half-filled with Pseudomonas Minimal Salts Medium (PMS). The tops of the bottles were loosely capped and secured with tape. The bottles were placed on a rotary shaker (200 rpm) in a 27° C. chamber and allowed to grow for 7 days prior to harvest.

### C. Pseudomonas Minimal Salts Medium (PMS)

The PMS medium used was developed by Gasson (Applied and Environmental Microbiology (1980). 39:25-29, as modified by Bolton (Bolton et al., Plant and Soil, 114: 279-287, 1989). The medium is made as follows: Dissolve 0.2 g potassium chloride, 1.0 g ammonium phosphate, 2.0 g sodium phosphate (monobasic), and 4.96 g sodium phosphate (dibasic) in distilled water to make 1 liter final volume. The medium is sterilized in an autoclave and allowed to cool, and then 2 ml of a sterile solution of 20% (w/v) magnesium sulfate (heptahydrate) and 20 ml of a sterile 10% (w/v) glucose solution is added per liter of autoclaved medium. For Fe containing medium, 2 ml/liter of a filter-sterilized solution of 1 mM FeCl₃ dissolved in 10 mM HCl was also added to the autoclaved medium.

### D. Preparation of Culture Filtrates

Pseudomonas isolates were grown in PMS medium (see above) for 7 days. The cultures were centrifuged (3,000×g, 15 minutes), and the supernatant was passed through a bacteriological filter (Millipore GP Express® Steritop®, 0.22 μm pore size). The resulting sterile culture filtrates were stored at 4° C.

### E. Solvent Extraction of Dried Culture Filtrates

Measured volumes of culture filtrate from Pseudomonas fluorescens WH6 (or other bacterial strains as indicated in the text) were taken to dryness in vacuo at 45° C. The evaporation flask was selected to have a volume at least ten-times that of the aliquot of culture filtrate to be evaporated. The dry solids recovered from the culture filtrate were extracted three times (5 minutes per extraction) with 90% (v/v) ethanol (or other test solvents as indicated in the text). For each of these three extractions, the dry solids were swirled with a volume of solvent equal to one-third of the original volume of culture filtrate. The three extracts prepared in this manner were combined and either stored at 4° C. for later use or immediately taken to dryness in vacuo at 45° C. and redissolved in a solvent appropriate to the planned experiment.

### F. Surface Sterilization of Seed

The seed of Poa annua (or other test seeds) were surface-sterilized by placing two to five grams of seeds in a 50 ml glass beaker, covering the seeds with a 50% (v/v) solution of H₂SO₄, and stirring vigorously for 5 minutes. The seed/acid mixture was filtered through a Gooch crucible and repeatedly rinsed with tap water to remove residual acid. The seeds were then placed in a beaker and covered with bleach (Clorox®, NaOCl, 5.25%) containing 1% (v/v) polyoxethylene sorbitan monolaurate (Tween®-20). The seeds were stirred for 5 minutes and then repeatedly rinsed with sterile deionized water and placed in a sterile Petri dish. The seeds were used immediately while wet or dried (spread in a thin layer and allowed to dry under a laminar flow hood with the Petri dish lid ajar for 4 to 8 hours) to prevent premature germination.

### G. Standard GAF Bioassay System

Bioassays for GAF activity were performed with Poa annua (annual bluegrass) seeds unless otherwise indicated. For each concentration of each test solution, 200 μl aliquots of solution were distributed to each of three wells of a sterile Costar® forty-eight well tissue culture plate (Corning® Costar® Number 3548). Three surface-sterilized Poa annua seeds were aseptically transferred to each well and submerged in the liquid, providing a total of nine observations for each concentration of each solution tested. The lid of the tissue culture plate was sealed with Parafilm® and the plate was placed in a germinator at 20° C. (8 hour day length at a light intensity of 50 μmol/m²/s, 16 hour dark period) for seven days. Germination was scored on the basis of the following four-point system:
Score 0: No visible signs of germination.
Score 0.5: Seed coat split but plumule and coleorhiza are not visible.
Score 1.0: Plumule and coleorhiza visible. Plumule chlorotic and shorter than length of seed. Root not yet visible.
Score 1.5: Plumule still shorter than length of seed, but root is clearly visible.
Score 2.0: Plumule approximately equal in length to the seed. Root elongated.
Score 2.5: Plumule obviously longer than length of seed. First true leaf still enclosed in coleoptile.
Score 3.0: First true leaf emerged from coleoptile and green. Emerged portion of the leaf is obviously shorter than the coleoptile.
Score 3.5: First true leaf emerged from coleoptile and green. Emerged portion of the leaf is approximately equal in length to the coleoptile.
Score 4.0: First true leaf emerged from coleoptile and green. Emerged portion of the leaf is obviously longer than the coleoptile.

### H. Soil-Based Tests for GAF Activity.

Soil-based tests for GAF activity were performed as follows: Woodburn sandy loam obtained from a Willamette Valley site was used in all soil-based experiments. The soil was dried for two days at 100° F. and screened through a ⅛-inch mesh prior to use. All flats were lined with black ground cloth. Small half-flats (25×25 cm) were filled with 1200 grams of soil. Larger flats (25×50 cm) were filled with 2500 grams of soil. The soil in the flats was fully saturated with water and left to drain in a greenhouse mist chamber for two days prior to seeding.

Soil-based tests involving only Poa annua (annual bluegrass) were conducted in half-flats. A frame covering ½- inch around the margins of the flat was placed on the soil surface, and the soil area within the frame was seeded with 0.3 grams of Poa annua seeds (leaving a ½-inch unseeded border). The seeds were sprayed lightly with distilled water and placed in a growth chamber set at 20° C. with a photoperiod consisting of 8-hour days (50 μEinsteins) and 16-hour nights. The seeds were allowed to imbibe water for 48 hours prior to treatment. At the end of the imbibition period, the seeds and soil surface in each flat were sprayed with 50 mL of treatment solution. Twenty-four hours after the first treatment, the flats were sprayed a second time with 50 mL of treatment solution. The treatments were evaluated and photographic records taken 12 days after the initial treatment (14 days from seeding). During this period, the flats were sprayed with distilled water as needed (typically daily) to maintain moist conditions for growth.

Soil-based tests involving both Poa annua seeds and established perennial ryegrass (Lolium perenne) seedlings were conducted in full sized flats (25×50 cm). After preparation of the soil as described above, five rows of perennial ryegrass seed (0.19 grams seed/row) were planted parallel to the short axis of the flat. The flats were placed in a mist chamber in the greenhouse for one week, and then transferred to a greenhouse bench where they were watered once or twice daily (as needed) with a fogging nozzle for the next 13 days. The ryegrass seedlings were fertilized at the end of the first week on the greenhouse bench. The fertilizer (a commercial 20-18-20 mixture containing trace elements) was supplied as a solution at a concentration of 243 ppm nitrogen. A pipette was used to apply this solution to the root crowns of the seedlings at a rate of 50 mL per seedling row. Six days after fertilization (20 days from seeding), the flats were transferred back to the greenhouse mist chamber for 24 hours. At the end of that time, the spaces between the rows were seeded with Poa annua seeds (0.08 grams of seeds per each of the 4 spaces). The seeds were sprayed with distilled water, and the flats were placed in a growth chamber set at 20° C. with a photoperiod consisting of 8 hour days (50 μEinsteins) and 16 hour nights. The seeds were allowed to imbibe water for 48 hours prior to spraying the seeded areas of each flat with 50 mL per flat of the appropriate treatment solution, prepared as described above. Twenty-four hours after the first treatment, each flat was sprayed a second time with 50 mL of treatment solution. The treatments were evaluated and photographic records taken 12 days after the initial treatment (14 days from seeding with Poa annua). During this period, the flats were sprayed with distilled water as needed (typically daily) to maintain moist conditions for growth.

### Analytical TLC Chromatography

Aliquots of culture filtrates (from Pseudomonas fluorescens Wh6 wildtype cultures, or cultures of other GAF-producing strains of Pseudomonas) were each taken to dryness in vacuo at 45° C. The samples of dry solids recovered in this manner were each extracted three times with 90% (v/v) ethanol. Each of the three extractions was performed by swirling the sample with a volume of 90% ethanol equal to one-third of the original culture filtrate volume for 5 minutes per each extraction. The combined ethanol extracts from a given sample were dried in vacuo (45° C.) and redissolved in a volume of 76% (v/v) ethanol sufficient to give a chromatographic sample of approximately 20-fold (20×) concentration relative to the original culture filtrate.

For analytical TLC chromatography, 0.15 ml of the 20× concentrate was applied as a band across Avicel® Microcrystalline Cellulose TLC plates (250 μm thick, 5×20 cm). The chromatograms were developed over a distance of 15 cm with ethyl acetate:isopropanol:water (15:30:20) as the developing solvent. The chromatographic separations were performed in cylindrical chromatography tanks (6 cm×23 cm) containing 25 ml of the developing solvent.

For ninhydrin staining, the developed TLC chromatograms were sprayed with a solution of 0.25% (w/v) ninhydrin dissolved in 100 ml of 95% (v/v) ethanol containing 3.0 ml of glacial acetic acid and then heated in an oven at 80-90° C. for 15 minutes.

### J. Taxonomic Identification of Bacterial Isolates.

Samples of each of the twelve DRB isolates used in Example 5 were provided to Microcheck, Inc. (Northfield, Vt.) for taxonomic identification based on Fatty Acid Methyl Ester Analysis (FAME Analysis) and on sequencing of a 400+bp DNA region near the beginning of the 16S-rRNA gene. Fatty acid profiles were analyzed using Sherlock, Version 6.0, software. For the DNA sequencing data, contiguous sequences were assembled using CAP3 (Huang and Madan, 1999) and aligned using ClustalW (Chenna et al., 2003).

### K. Deposit of Reference Cultures of Pseudomonas Isolates

Isolates identified as GAF (FVG)-producing bacteria were cultured, lyophilized, and submitted to the ARS Patent Culture Collection for deposit under the conditions of the Budapest Treaty on the International Recognition of the Deposit of Microorganisms for the Purpose of Patent Procedures. The date of deposit of P. fluorescens isolates WH6 (NRRL# B-30485), AD31 (NRRL# B-30483), AH4 (NRRL#B-30482), E34 (NRRL# B-30481), and WH19 (NRRL# B-30484) was Jun. 13, 2001. The date of deposit for P. fluorescens isolates AH10 (NRRL# B-50232), BT1 (NRRL#B-50230), E24 (NRRL# B-50229), TR33 (NRRL# B-50220), TR44 (NRRL# B-50219TR46 (NRRL# B-50218), A3422A (NRRL# B-50234), ALW38 (NRRL# B-50231), G2Y (NRRL# B-50228), GTR12 (NRRL# B-50227), GTR24 (NRRL# B-50226), GTR40 (NRRL# B-50225), HB14 (NRRL# B-50224), HB26 (NRRL# B-50223), HB32 (NRRL# B-50222), ST22 (NRRL# B-50221), W36 (NRRL# B-50217), and Pseudomonas mucidolens/synxantha A342 (NRRL# B-50236) and TDH40 (NRRL# B-50235), and Enterobacter kobei A3203 (NRRL# B-50233) was Jan. 23, 2009.

### L. Genomic DNA Isolation, Primer Design and Standard PCR Conditions.

DNA was isolated from pure cultures of bacteria using the ZR Fungal/Bacterial DNA Kit (Zymo Research, Orange, Calif.) and stored at 4° C. until required. To create a PCR detection test specific for GAF-producing organisms, PCR primer sequences were selected to anneal to unique regions of the GAF2-formyl-transferase gene of P. fluorescens WH6. Detection primers were designed to span a 316 nucleotide region within the gene, consisting of forward primer

| | |
|---|---|
| 5′GACAATCGAGCCATGCAA3′ | (SEQ ID NO: 3) |

and reverse primer

| | |
|---|---|
| 5′GCCGTAGGTTTCATCGTTGT3′. | (SEQ ID NO: 4) |

All PCR amplifications were conducted in a Robocycler (Stratagene, La Jolla, Calif.) thermocycler, and reactions were carried out according to the Platinum Taq polymerase manufacturer's instructions (Invitrogen, Carlsbad, Calif.). Thermocycling consisted of initial denaturation for 5 min at 94° C. followed by 40 cycles (30 sec, 94° C.; 30 sec, 56°C.; 30 sec, 72° C.), with a final 10-minute extension step at 72° C. The samples were subjected to electrophoresis through agarose gels containing 10 μg/mL of ethidium bromide and amplicons visualized by exposure to a UV light.

### Example 2

### Structure Elucidation of the Germination-Arrest Factor (GAF)

### General Experimental Procedures.

NMR data were acquired on a Bruker DRX 300 MHz spectrometer with a 5 mm BBO probe. Low resolution mass spectra were acquired on a Finnigan® LCQ Classic Ion Trap Mass

Spectrometer (ES+) or a Waters® Micromass LCT mass spectrometer (TOF-ESI). HRCI+ mass data were acquired on a JEOL® MSRoute spectrometer. Optical rotations were measured in a microcell (160 μL) on a JASCO® P-1010 polarimeter. For NMR data acquisition, two purified GAF sample solutions (DA-IV-19 and DA-IV-21 in 76% ethanol) were combined and concentrated to dryness in vacuo (6 hr, ˜37° C.) for an estimated actual mass of 0.4 mg GAF. This sample was taken up in 99.9% deuterated water (D₂O, 460 μL), placed in a standard 5 mm NMR tube, and used to acquire ¹H, ¹³C, COSY, multiplicity-edited HSQC and HMBC (d6=65 ms) experiments (referenced to external DSS). To acquire additional NMR and also optical rotation data, a second, larger sample of GAF, (purified using the same procedures) was dried to give an apparent mass of 3.6 mg (likely to include inorganic salts) and prepared for NMR analysis (D₂O, 450 μL). A ¹H NMR spectrum showed that this second sample was pure enough to carry out the additional NMR experiments needed to distinguish the two proposed structures described below and to assign geometry. An aliquot of this sample was subsequently dried down, weighed (1.3 mg) and reconstituted in 3:1 water-ethanol (400 μL) for polarimetry.

### Isolation of GAF

Previous work, documented in PCT/US03/38653 (published as WO 04/052097; incorporated herein by reference in its entirety), established that GAF activity was associated with a particular ninhydrin-reactive band visible on cellulose thin-layer chromatography (TLC) plates when ethanol extracts of solids recovered from culture filtrates of P. fluorescens WH6 from other GAF-producing strains of bacteria were chromatographed on TLC plates in neutral solvent systems.

For purification of GAF, aliquots (150 mL) of culture filtrates from P. fluorescens WH6 (grown in Pseudomonas Minimal Salt Medium) were evaporated to dryness in vacuo at temperatures ≦45° C. The dry solids were extracted by swirling with 90% (v/v) ethanol (three successive 50 mL volumes of this solvent, 5 minutes per extraction). The extracts were combined, dried in vacuo (≦45° C.), and the recovered solids dissolved in 7.5 ml of 76% (v/v) ethanol to give a solution of 20× concentration relative to the original culture filtrate. The 76% ethanol solutions were stored at 4° C. prior to further processing.

Aliquots of the GAF-containing 76% ethanol solutions described above were chromatographed on Analtech Microcrystalline Cellulose Preparative Thin-Layer Chromatography (TLC) Plates (20×20 cm, 1000 μM thick). The plates were prewashed by ascending chromatography, twice in distilled water and once in redistilled 95% ethanol, prior to use. A 3.2 mL aliquot of the 20× GAF extract was applied to each prewashed TLC plate, as a band located 3 cm from one end of the TLC plate, using an Analtech TLC Sample Streaker equipped with a 500 μL syringe, and set at the maximum delivery angle. At this angle, 80 μL was delivered at each traverse of the plate. The sample streak was dried in a cool air stream after every two traverses, and applications were repeated until the total 3.2 mL sample (equivalent to 64 mL of original culture filtrate) had been applied to the plate. The preparative chromatograms were developed in rectangular tanks (7½27½ base×24 cm deep) containing 192 mL of freshly mixed solvent consisting of ethyl acetate (30 mL), isopropanol (90 mL), and deionized water (72 mL). The chromatograms were developed over 12 cm and allowed to dry for 35-45 minutes before examining under long-wave UV light. Previous work has established that under these conditions GAF chromatographed at a position bounded on the lower side by a bright blue fluorescent band and on the upper side by a very narrow and weak blue fluorescent band that were visible under these lighting conditions. Using these markers, the position of the GAF band was marked with pencil

The cellulose from the TLC zone corresponding to GAF was scraped into a 30 mL Corex centrifuge tube. Deionized water (16 mL/tube) was added, and the tubes were vortexed for 3 minutes, and centrifuged (Sorvall® SS-34 rotor, 7500 rpm=6,780×g) for 10 minutes. The pellet was discarded, and the supernatant was filtered through a 0.2 micron bacteriological filter (Acrodisc® 25 mm Syringe Filter, Pall Corporation) into a sterile sample bottle. The resulting sterile aqueous solutions (4× concentration relative to the original culture filtrate) were stored at 4° C. until further processing. Analytical TLC analysis of this solution demonstrated that it contained a single ninhydrin-reactive compound with the Rf expected of GAF.

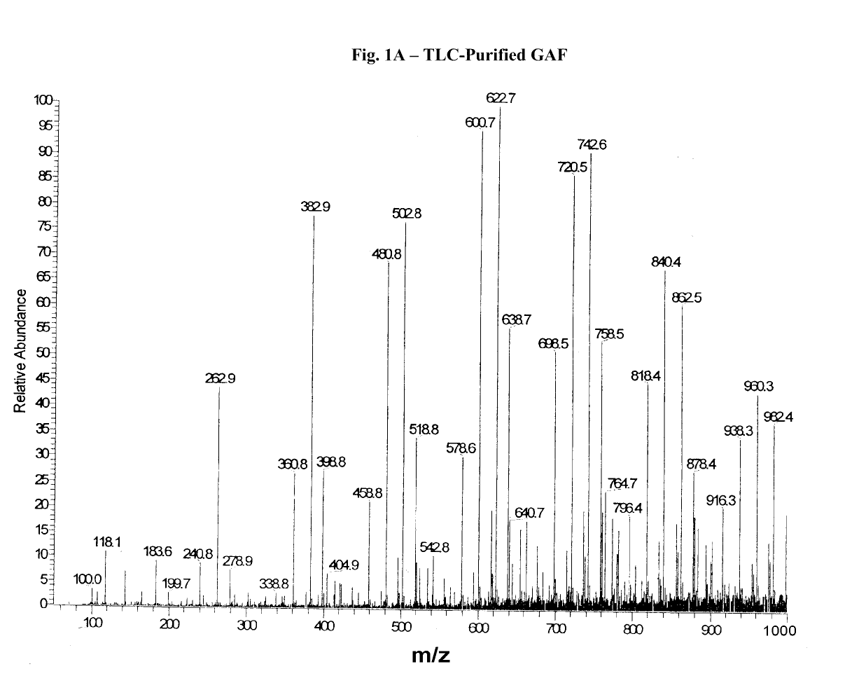

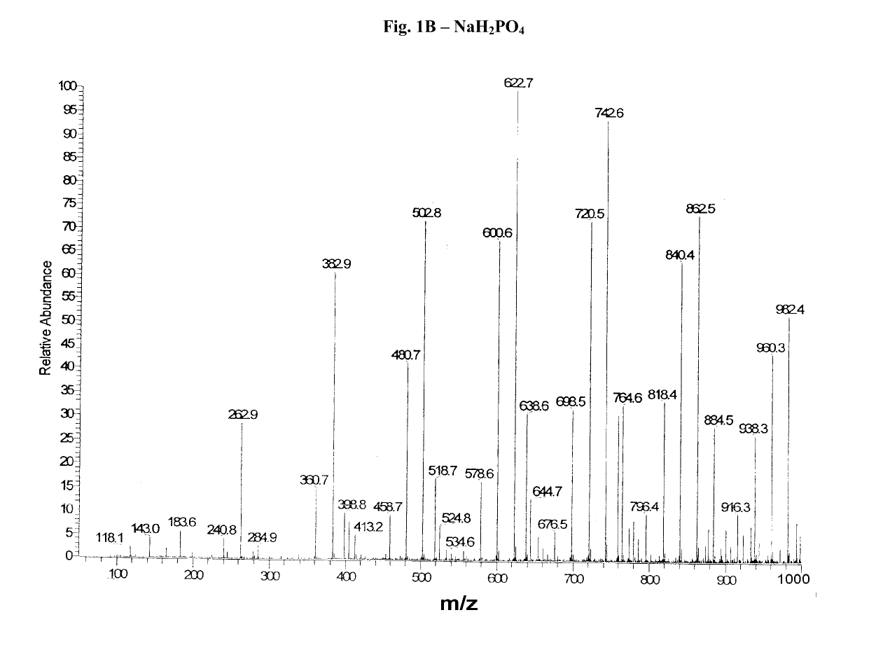

The purity of the GAF preparations recovered from preparative thin-layer chromatograms was assessed by mass spectrometric analysis using the facilities and services of the Oregon State University Mass Spectrometry Facility. Samples for mass spectrometry were prepared by drying aliquots of the 4× aqueous GAF solutions (in vacuo , ≦45° C.) and redissolving the recovered solids in a volume of 60% (v/v) ethanol that gave a resulting 80× GAF concentration. These samples were analyzed on a Finnigan® LCQ Classic Ion Trap Mass Spectrometer using electrospray (ES) injection. The samples were acidified by the addition of an equal volume of 0.1% formic acid immediately before injection into the mass spectrometer. A representative mass spectrum obtained in this manner is shown in FIG. 1A. Doublets in which the individual doublet peaks were separated by 22 mass units (presumably representing addition or subtraction of sodium) were observed as repeating signals separated by 120 mass units and occurring up to m/z of at least 1000. While this was at first taken to represent the breakdown of a polymer, a virtually identical pattern was obtained when a solution of pure NaH₂PO₄ was injected into the mass spectrometer (FIG. 1B). Thus, the repeating structure of the mass spectrum was actually due to NaH₂PO₄ aggregates of varying size that were generated under ES conditions. Although this result provided no information concerning the structure of GAF, it did indicate that the major contaminant present in our TLC-purified GAF preparations was inorganic phosphate. That conclusion caused us to focus our attention on possible ways of separating GAF from contaminating phosphate.

Ion exclusion chromatography on Sephadex® offers one possibility for separating the weakly ionic GAF molecule from the strongly charged phosphate ion. In distilled water (i.e. in the absence of a buffer), the negative charges on the bead surface will tend to repel anions and cause them to elute earlier from the column than might be expected solely on the basis of their size. One of the advantages offered by this approach was that it did not introduce buffers or other potential contaminant molecules into the sample. However, the use of bioassay procedures to determine the distribution of GAF in fractions generated by column chromatography promised to be cumbersome and time consuming, and a method was also needed to assess the distribution of phosphate in these fractions. A possible solution to both of these problems was suggested by results obtained in earlier work on the interaction of GAF with trace metals.

We had earlier considered the possibility that GAF might be one of the iron-binding compounds known as siderophores. Although many strains of Pseudomonas fluorescens produce siderophores, these compounds are normally only produced under conditions of iron deficiency. Because our PMS medium had been modified to contain iron, it appeared unlikely that GAF would have the properties of a siderophore. Nevertheless, we attempted to determine whether interactions between GAF and various trace metals might be detected using the Poa bioassay. The results of these experiments are summarized in Table 1 (shown on the next page). With the exception of calcium, all of the trace metals tested were at least somewhat toxic at the higher concentrations tested (up to 200 μM). Iron exhibited slight toxicity at the higher concentrations (50 to 200 μM), but no evidence of interaction with GAF could be detected in the bioassay. Copper, on the other hand, was the most toxic of the trace elements tested, completely inhibiting germination at the higher concentrations, but the toxicity of this metal was significantly alleviated in the presence of GAF. Similarly, the activity of GAF in arresting germination was greatly reduced in the presence of appropriate concentrations of copper ions. Among the trace metals tested (copper, iron, nickel, calcium, and zinc), copper was unique in exhibiting this sort of interaction with GAF. This result suggested that we might be able to use copper-binding by GAF as the basis of a colorimetric assay for the GAF molecule, at least in those cases where GAF had already been partially purified, concentrated, and separated from likely interfering compounds.

TABLE 1
Interactions of GAF with Trace Metals in the Standard Poa Bioassay.

| | GAF | POA BIOASSAY SCORES (±Standard Error of the Mean) | | | | | |
|---|---|---|---|---|---|---|---|
| | Conc. | At the Indicated METAL CONCENTRATION (μM) | | | | | |
| METAL | (X)* | 0 μM | 12.5 μM | 25 μM | 50 μM | 100 μM | 200 μM |
| CuCl₂ | 0 | 4.00 ± 0.00 | 3.3 ± 0.17 | 2.9 ± 0.04 | 1.2 ± 0.27 | 0.30 ± 0.28 | 0.00 ± 0.00 |
| | 2X | 1.00 ± 0.00 | 1.00 ± 0.00 | 2.58 ± 0.06 | 3.94 ± 0.06 | 2.97 ± 0.03 | 2.69 ± 0.09 |
| CuSO₄ | 0 | 4.00 ± 0.00 | 3.5 ± 0.00 | 3.00 ± 0.00 | 1.11 ± 0.11 | 0.00 ± 0.00 | 0.00 ± 0.00 |
| | 2X | 1.00 ± 0.00 | 1.00 ± 0.00 | 1.00 ± 0.00 | 1.00 ± 0.00 | 3.00 ± 0.00 | 0.00 ± 0.00 |
| FeSO₄ | 0 | 4.00 ± 0.00 | 4.00 ± 0.00 | 3.50 ± 0.13 | 3.00 ± 0.00 | 2.86 ± 0.44 | 2.47 ± 0.07 |
| | 2X | 1.00 ± 0.00 | 1.00 ± 0.00 | 1.00 ± 0.00 | 1.00 ± 0.00 | 1.00 ± 0.00 | 1.00 ± 0.00 |
| NiCl₂ | 0 | 4.00 ± 0.00 | 2.78 ± 0.05 | 2.47 ± 0.10 | 1.78 ± 0.09 | 1.44 ± 0.06 | 1.00 ± 0.00 |
| | 2X | 1.00 ± 0.00 | 1.00 ± 0.00 | 1.00 ± 0.00 | 1.00 ± 0.00 | 1.00 ± 0.00 | 1.00 ± 0.00 |
| CaCl₂ | 0 | 4.00 ± 0.00 | 4.00 ± 0.00 | 4.00 ± 0.00 | 4.00 ± 0.00 | 4.00 ± 0.00 | 4.00 ± 0.00 |
| | 2X | 1.00 ± 0.00 | 1.00 ± 0.00 | 1.00 ± 0.00 | 1.00 ± 0.00 | 1.00 ± 0.00 | 1.00 ± 0.00 |
| ZnCl₂ | 0 | 4.00 ± 0.00 | 4.00 ± 0.00 | 4.00 ± 0.00 | 4.00 ± 0.00 | 3.5 ± 0.00 | 3.00 ± 0.00 |
| | 2X | 1.00 ± 0.00 | 1.00 ± 0.00 | 1.00 ± 0.00 | 1.00 ± 0.00 | 1.00 ± 0.00 | 1.00 ± 0.00 |

*GAF concentration is expressed in units relative to the concentration in original culture filtrate (defined as a concentration of 1X). The GAF preparation used was partially purified by 90% ethanol extraction of dried culture filtrate followed by preparative thin-layer chromatography. For purposes of this comparison, recovery of GAF activity relative to the culture filtrate is assumed to be 100%.

Chrome Azurol S (5-((3-carboxy-5-methyl-4-oxo-2,5-cyclohexadien-1-ylidene)-(2,6-dichloro-3-sulfophenyl)methyl)-2-hydroxy- 3-methylbenzoic acid trisodium salt) is a weak chelator of iron and forms a highly colored complex with that metal and other transition metal elements. Strong chelating agents are able to strip iron from Fe-Chrome Azurol S complexes with a resulting change in absorbance that has been used to measure siderophore activity (Langmyhr & Klausen, Anal. Chim. Acta 29: 149-167, 1963). However, in tests for siderophore activity using Fe-Chrome Azurol S, a variety of iron-binding compounds are known to give false positives, including phosphate buffers. Copper can also bind to Chrome Azurol to give a colored complex with an absorption spectrum only slightly different from that of the corresponding iron complex. The Cu-Chrome Azurol complex has also been used to detect and measure chelating activity (Shenker et al., Soil Sci. Soc. Am. J. 59: 1612-1618, 1995). The apparent interaction of copper with GAF in our biological tests and the absence of any evidence for an interaction with iron led us to test whether Cu-Chrome Azurol S could be used to detect GAF in fractions generated by chromatography of TLC-purified GAF on Sephadex® columns.

The Chrome Azurol S reagents were prepared according to the Shenker et al. reference cited above. The reagents were composed of 210 μM Chrome Azurol S and 200 μM of either CuSO₄ or FeSO₄, in 40 mM MES buffer. The resulting solutions were adjusted to either pH 5.5 (Cu-ChromeAzurol) or 5.7 (Fe-ChromeAzurol) with NaOH. Chrome Azurol S and MES [2-(N-morpholino)-ethanesulfonic acid] were purchased from Sigma-Aldrich.

To assay column fractions for metal binding, 75 μL of the appropriate Chrome Azurol reagent were mixed with 75 μL of column fraction in one of the wells of a 96-well EIA/RIA plate (Corning Costar No. 3590). The plates were sealed with Parafilm® and the color changes allowed to develop overnight. Absorbance in the wells was measured at 595 nm in a Bio-Tek EL808 Plate Reader. For graphical purposes, the changes in absorbance were calculated by subtracting the measured absorbance in each well from the highest well absorbance measured on a given plate. These values were plotted as A595 Change.

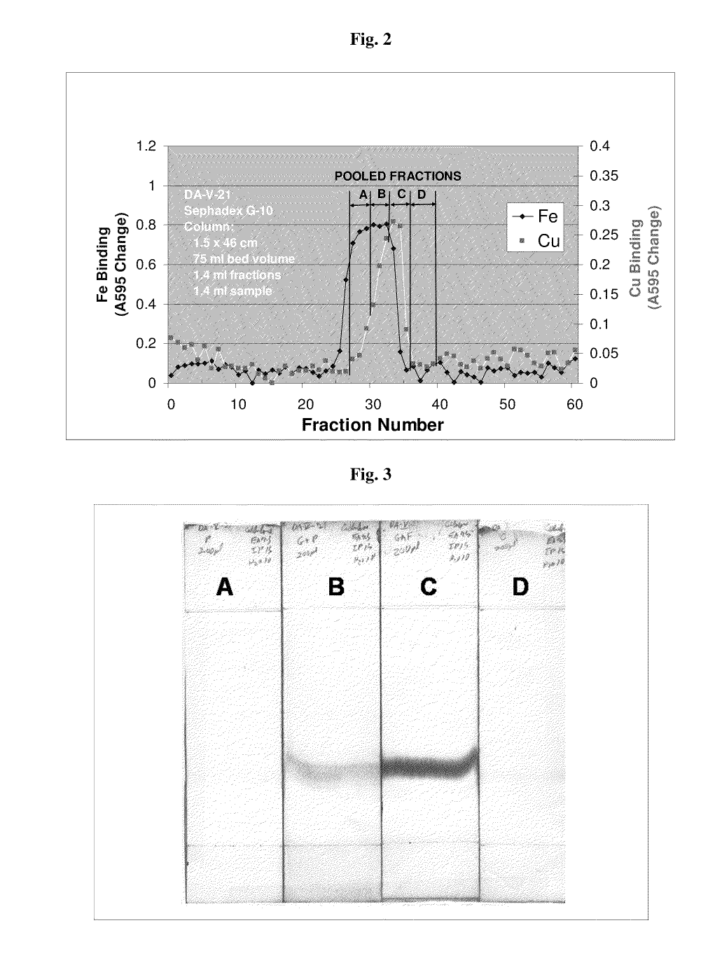

A sample of TLC-purified GAF, concentrated to 40×, was chromatographed on a Sephadex® G10 column in deionized water as shown in FIG. 2. The absorbance profile obtained by assaying the resulting column fractions with the Cu-Chrome Azurol reagent exhibited a peak that was shifted to the right (later eluting fractions) relative to a broad absorbance profile obtained using the Fe-Chrome Azurol reagent (the latter was presumed to reflect the distribution of phosphate ions in the profile.) After pooling and concentrating the fractions as indicated, TLC analysis of the pooled fractions indicated that the ninhydrin-positive band corresponding to GAF tracked with the copper-binding peak (FIG. 3). These results indicated that the Cu-Chrome Azurol reagent could be used to track GAF, at least in these relatively concentrated and partially purified solutions, and that some separation of GAF from contaminating phosphate was possible using the differences in ion exclusion on Sephadex® columns in distilled water.

The optimal Sephadex® chromatographic conditions for separation of phosphate and GAF in TLC-purified GAF preparations were explored in a series of subsequent experiments. On the basis of these experiments, chromatography on Sephadex® G15 columns under carefully defined sample volume/bed volume ratios currently is believed to be the most cost-effective and efficient method for affecting this separation.

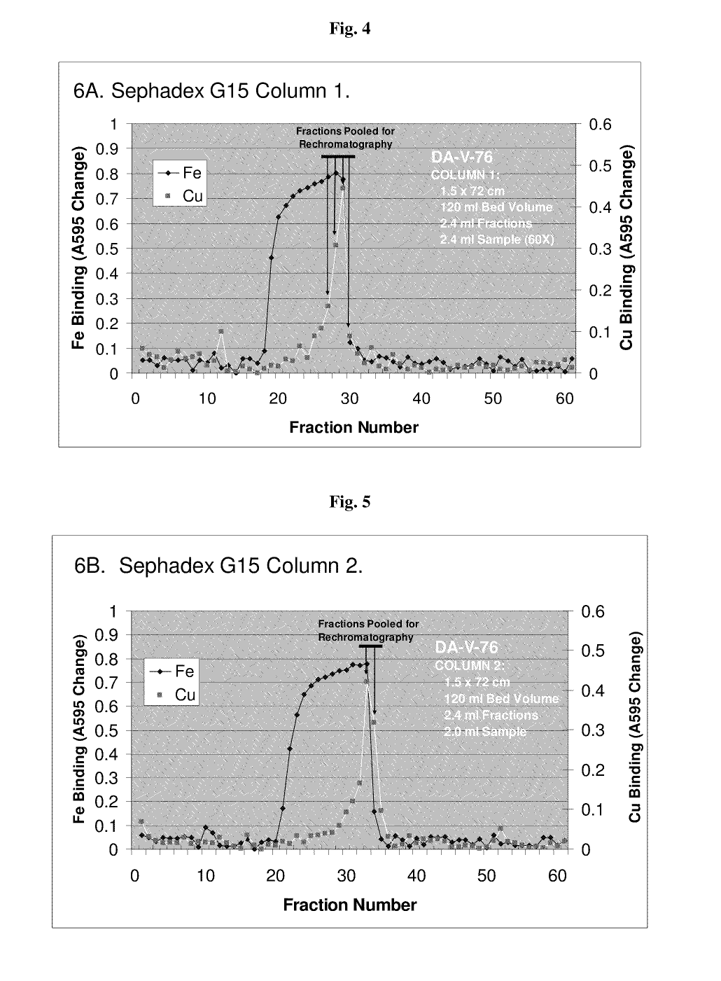

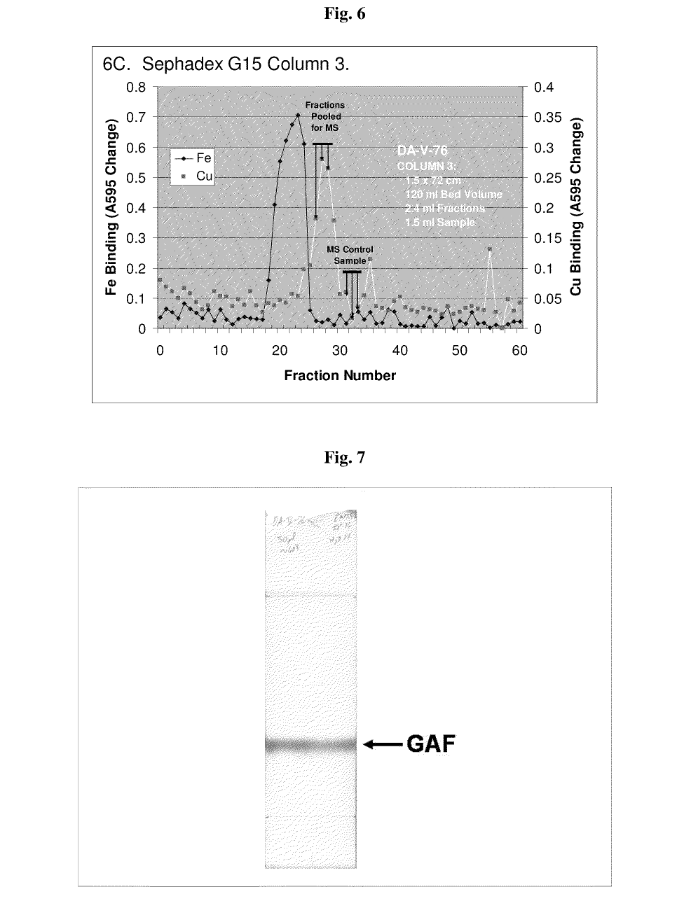

To obtain a purified preparation of GAF sufficient for mass spectrometric analysis, a concentrated sample of TLC-purified GAF (equivalent to 144 mL of original WH6 culture filtrate) was chromatographed on a Sephadex® G15 column as illustrated in FIG. 4. Fractions corresponding to the copper-binding peak were pooled and rechromatographed on two additional consecutive G15 columns as shown in FIGS. 5 and 6. The resulting purified GAF preparation was dissolved in a small volume (1.2 mL) of 60% ethanol for mass spectrometric analysis. This preparation yielded a single sharp ninhydrin-staining band when analyzed by analytical TLC chromatography as shown in FIG. 7. A control sample was taken by pooling a corresponding number of fractions from regions of the profile corresponding to the base line obtained after elution of the copper-binding peak.

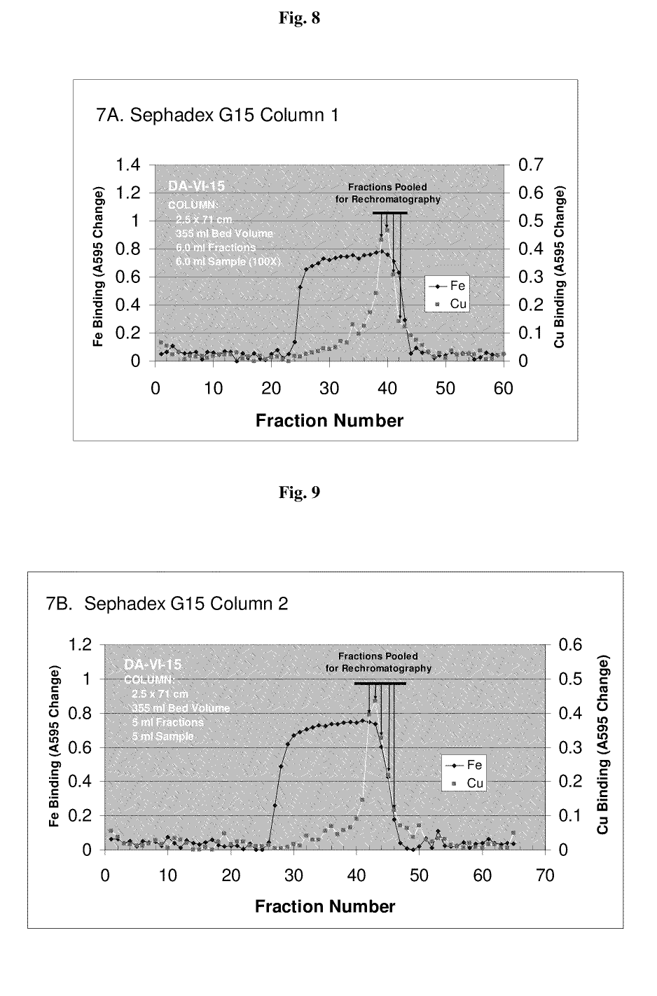

The preparation of a purified GAF sample for NMR analysis required a further scale-up of the purification procedure. For this purpose, two 150 mL samples of TLC-purified GAF (4× concentration, each equivalent to 600 mL of WH6 culture filtrate) were concentrated and chromatographed separately on appropriately scaled Sephadex® G15 columns as shown in FIGS. 8-10. The purified GAF samples were stored at 4° C. prior to being combined and concentrated for NMR spectroscopy as described above. To acquire additional NMR data needed to confirm the preliminary molecular structure and assign double bond geometry, a second sample of GAF was purified using the same procedures but beginning with a larger volume of WH6 culture filtrate. The chromatographic data for this purification are not presented here, but three 150 mL aliquots of TLC-purified GAF (4× concentration, each equivalent to 600 mL culture filtrate and the total equivalent to 1800 mL of culture filtrate) were processed by recycling on Sephadex® G15 columns in the same manner illustrated in FIGS. 8-10. The NMR data showed that this second sample was less pure than the first, but pure enough to confirm the presence of the same major compound (GAF) and to carry out the additional NMR experiments needed as described herein.

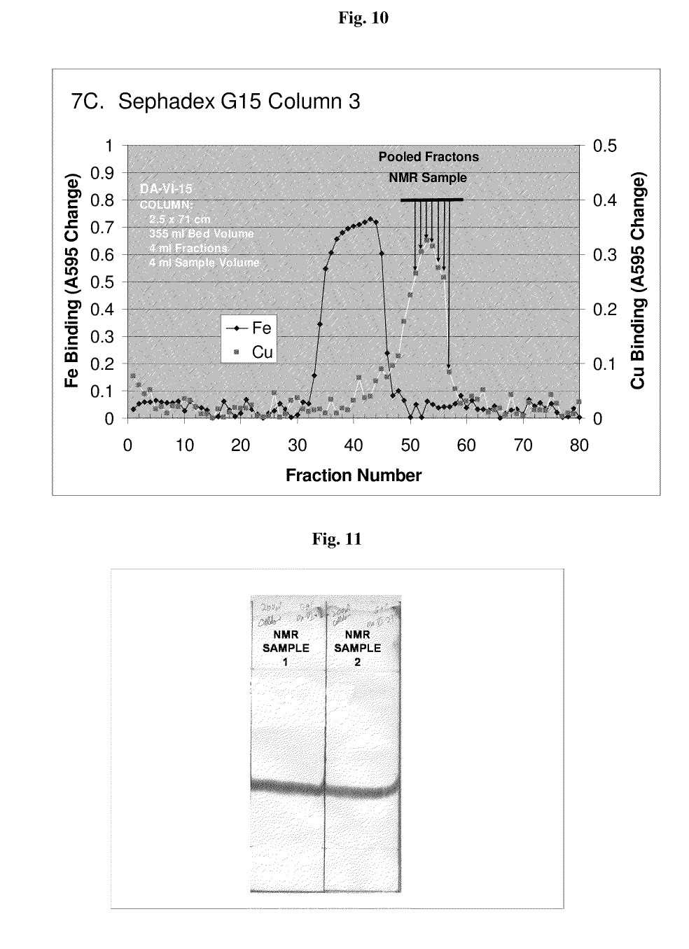

The recovery of biological activity from chromatography was determined for one of these samples by taking an aliquot of the aqueous pooled fractions from the final column purification of that sample (FIG. 10). The bioassay results are summarized in Table 2:

TABLE 2

| SAMPLE CONCENTRATION | GERMINATION |
|---|---|
| (% Pooled Volume Concentration) | SCORE |
| Undiluted Pooled Volume (100%) | 1.00 |
| 10% | 1.00 |
| 5% | 1.00 |
| 2.5% | 1.00 |
| 1.25% | 1.92 |
| 0% | 4.00 |

With reference to Table 2, after pooling and concentrating the GAF-containing fractions from each purification, small aliquots (200 μL each) of the two resulting purified GAF samples (each redissolved in 10 mL 76% ethanol) were taken for TLC analysis. Single sharp ninhydrin positive bands were observed with each sample as shown in FIG. 11.

### Structural Elucidation of GAF.

Extracts of Pseudomonas fluorescens isolate WH6 possessed significant herbicidal activity against grassy weeds, and were purified as described above to yield the active component designated as Germination Arrest Factor (GAF, 2-amino-4-formylaminooxy-but-3-enoic acid): [α]_{D} +21.8 (c 0.03, CHCl₃) ; HR CIMS(+) obsd [M+H]⁺ m/z 161.05195 (calcd for C₅H₉O₄N₂, 161.05624).

Carbon and proton positions are designated as follows:

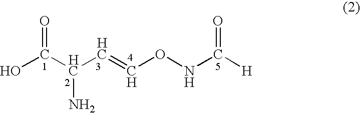

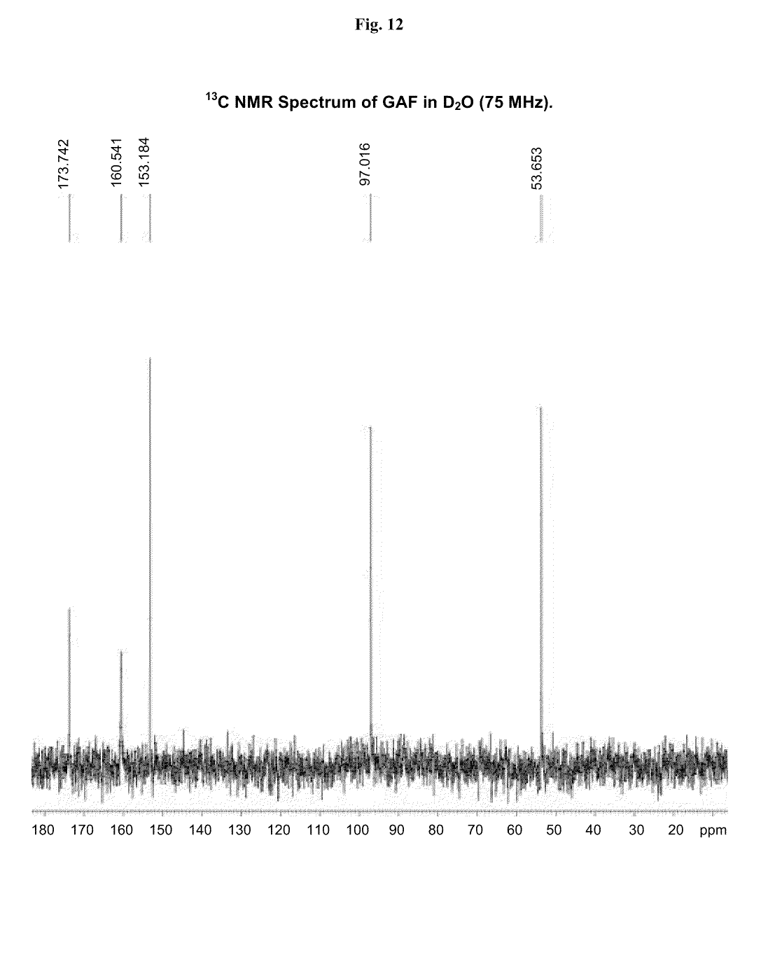

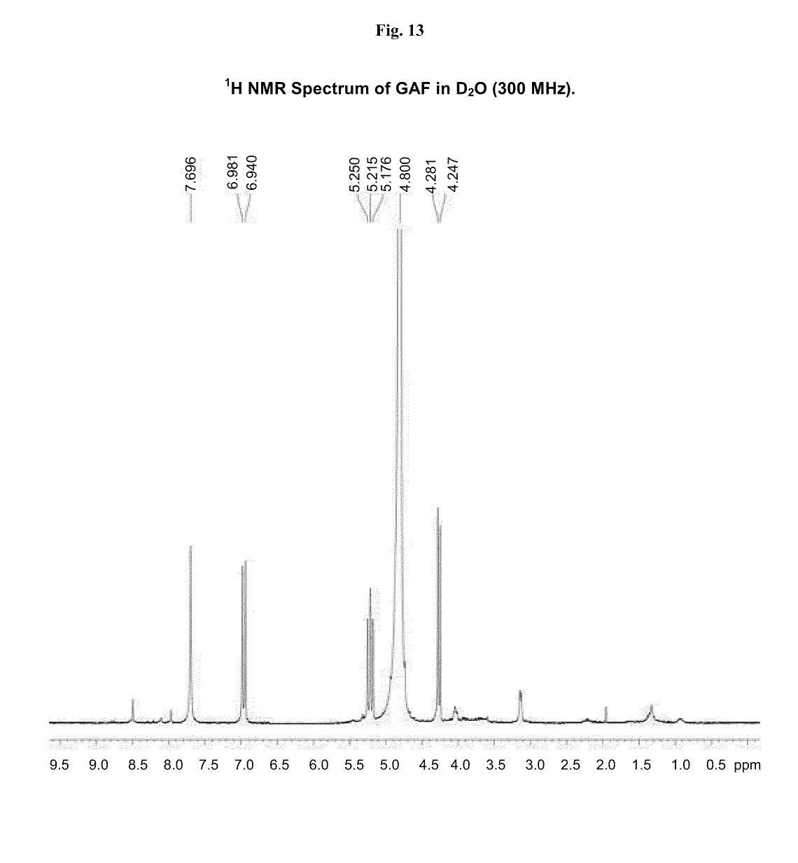

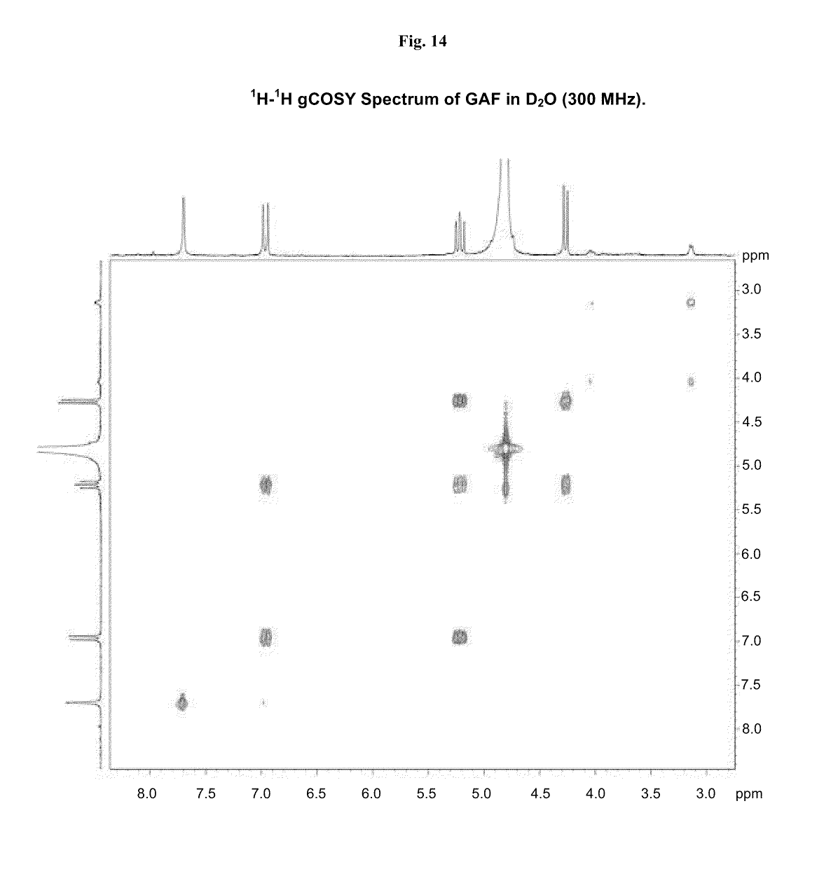

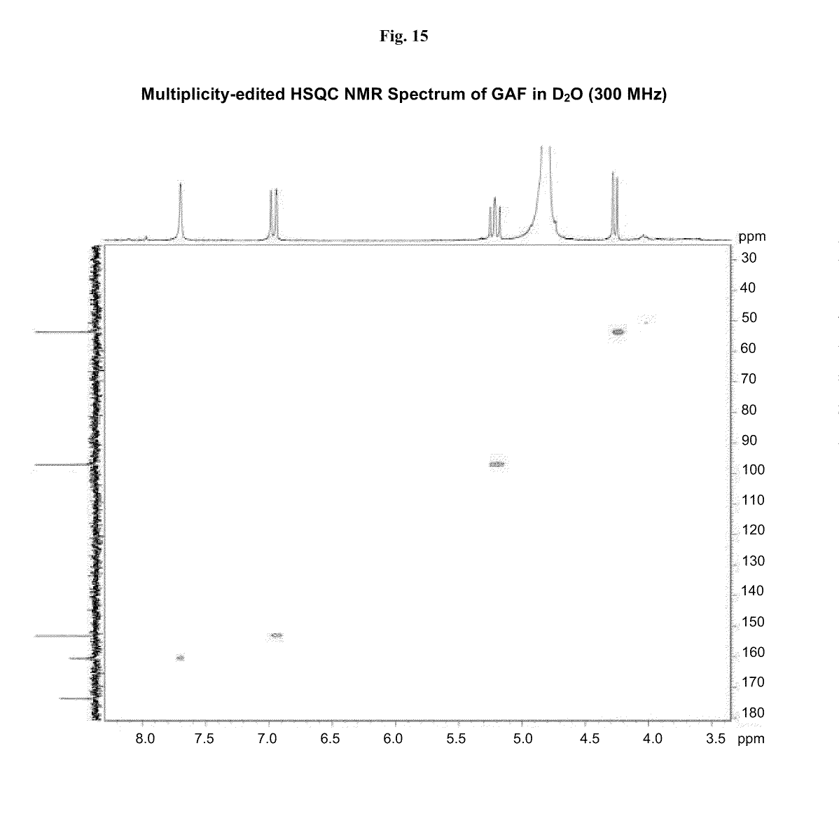

Structural characterization of the highly functionalized vinylglycine GAF (2) initially was impeded by its polarity, reactivity and low molecular weight, which hindered the use of mass spectrometry to gain an initial, definitive low resolution estimate of molecular size. However, the ¹³C NMR data for GAF (FIG. 12) were consistent with a small molecule possessing five carbons, and the ¹H NMR spectrum (FIG. 13) showed only four resonances, all of equal integration. The latter signals comprised a singlet (δ_{II} 7.70), two doublets (δ_{II} 6.96 and 4.26) and a closely overlapped doublet of doublets (δ_{II} 5.22). Examination of their coupling constants (J_{II,II}) revealed coupling of each doublet to the doublet of doublets but not to each other, and this was confirmed from 2D COSY data (see Table 3 and FIG. 14). Therefore, the molecule contained a linear —CHCHCH— spin system as well as an isolated proton (δ_{H} 7.70, H-5). These relatively deshielded ¹H chemical shifts were consistent with the presence of four deshielded olefinic or heteroatom-substituted methine resonances (δ_{C} 53.7, 97.0, 153.2 and 160.5) in the ¹³C and DEPT135 NMR spectra. The remaining quaternary ¹³C resonance (δ_{C} 173.7, C-1) implied the presence of a carbonyl carbon. A multiplicity-edited gHSQC experiment (FIG. 15) allowed the assignment of protons and carbons as listed in Table 3, and confirmed the hybridization of each carbon.

TABLE 3
¹H and ¹³C NMR data for GAF (CDCl₃, 300 MHz, 298K)

| Atom | | | | |
|---|---|---|---|---|
| # | δ_{C} | δ_{H}, mult. (J/Hz) | COSY | HMBC |
| 1 | 173.7 C | — | — | — |
| 2 | 53.7 CH | 4.26, d (10.0) | 5.22 | 97.0, 153.2, 173.7 |
| 3 | 97.0 CH | 5.22, dd (12.2, 10.0) | 6.96, 4.26 | 53.7, 153.2, 173.7 |
| 4 | 153.2 CH | 6.96, d (12.2) | 5.22 | 53.7, 97.0 |
| 5 | 160.5 C | 7.70, s | — | — |

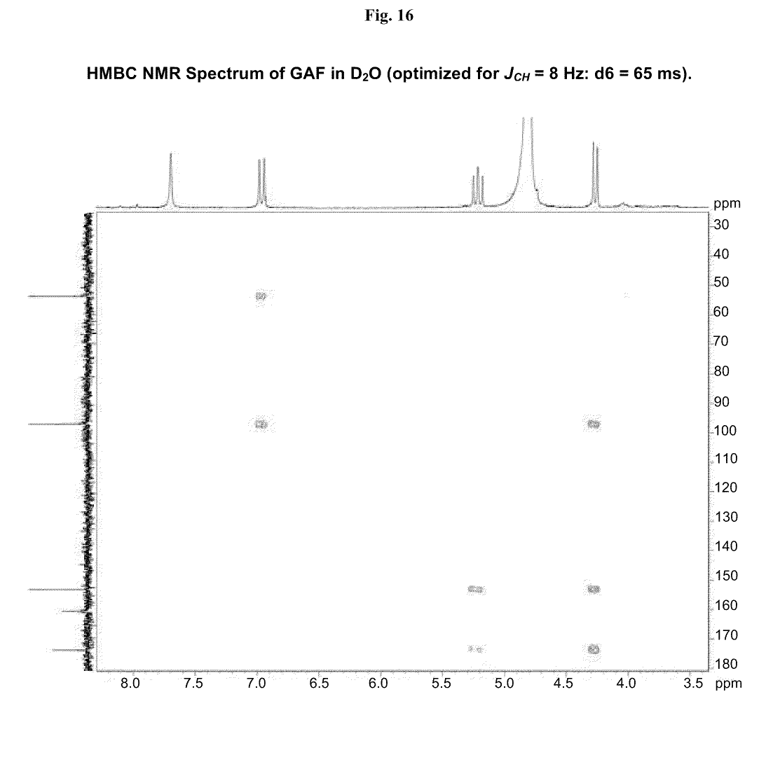

Long range H—C correlations were observed in a 2D gHMBC experiment (Table 3 and FIG. 16). Two and three bond HMBC correlations from H-2 (δ 4.26) to C-1, C-3 and C-4, and from H-3 to C-1, C-2 and C-4 suggested an alpha amino acid structure for GAF. Furthermore, in combination with COSY data and HMBC correlations from H-4 to C-2 and C-3, these data delineated a vinylglycine core (see partial structure A illustrated below; MW 100 Da). Thus it remained to assign the isolated methine (δ_{C} 160.5, δ_{H} 7.70, s) and NMR silent heteroatoms which would account for the observed (deshielded) ¹H and ¹³C chemical shifts, and which would have a molecular mass of 160 Da (see below). Considering that Pseudomonas peptidic secondary metabolites regularly incorporate N-formyl and N-hydroxy amino acid residues, two alternative vinyl substituents B and C could be proposed for GAF (see illustrated structures A, B and C below).

### Partial Structures for GAF:

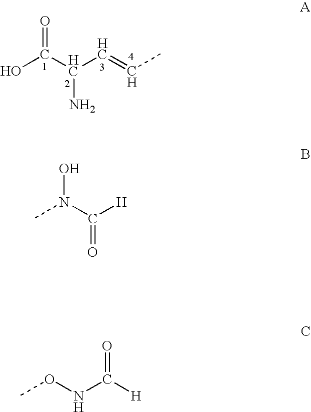

With reference to the partial structures illustrated above, A is a vinylglycine core; B is an N-hydroxyformyl substituent, and C is an N-oxyformyl substituent.

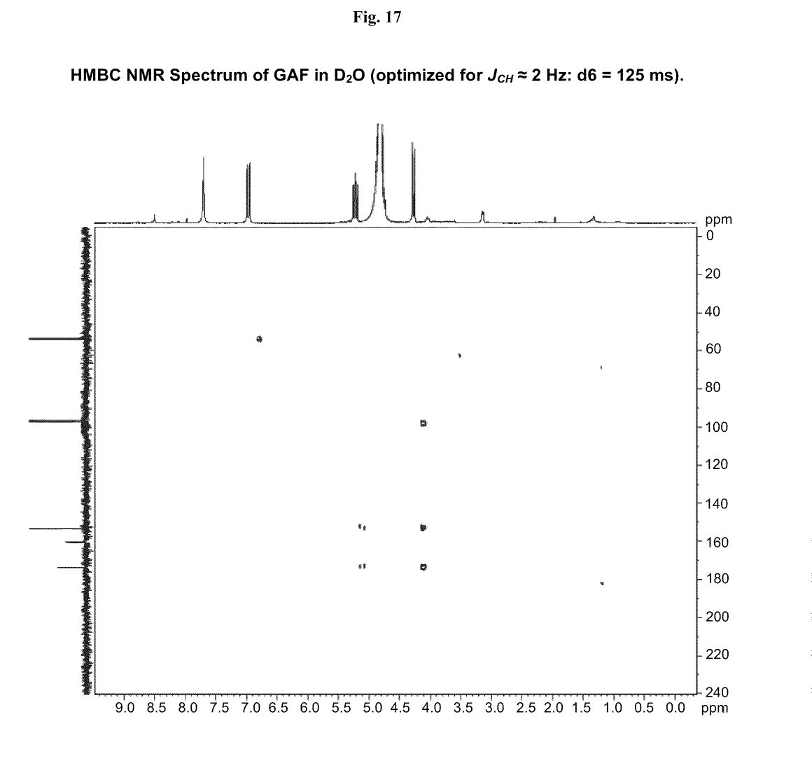

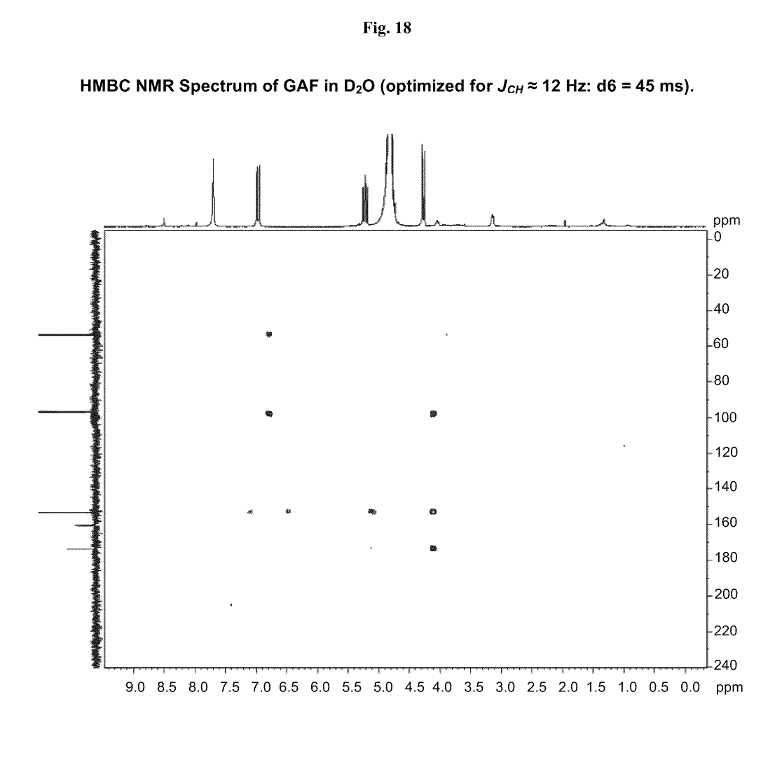

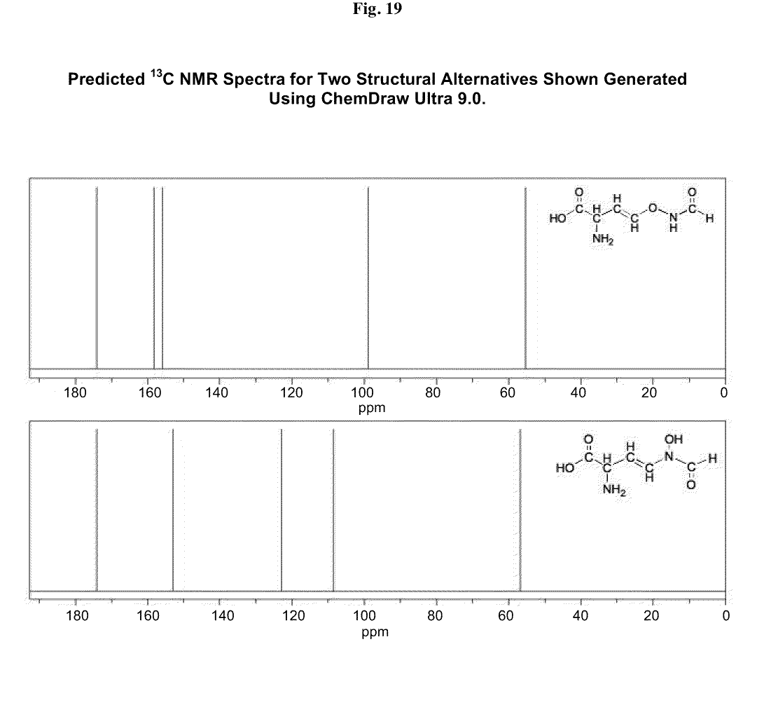

An aminovinyl linkage of partial structure A to substituent B to give molecular structure D (below) would present a 3-bond connectivity from H-4 to C-5, which should be evident in an appropriately optimized HMBC experiment. However, no correlation from H-4 to C-5 could be discerned in HMBC experiments optimized for a range of heteronuclear coupling constants (J_{H,C} 2-12 Hz, FIGS. 17 and 18). Indeed, the deshielded chemical shift of vinyl C-4 (δ 153.2) is more consistent with the oxyvinyl linkage of substituent C to give structure E (below). This represents a 4-bond connectivity between H-4 and C-5 which is usually inaccessible by HMBC analysis. In addition, ¹³C NMR predictions were performed using ChemDraw Ultra 9.0 and ACDLabs software. While ACDLabs predictions do not distinguish between the two compounds, ChemDraw predictions support the presence of an oxyvinyl linkage in GAF through a deshielded resonance for the oxygenated olefinic carbon (δ 155.9) versus that for the nitrogenated olefinic carbon (δ 122.9) (FIG. 19).

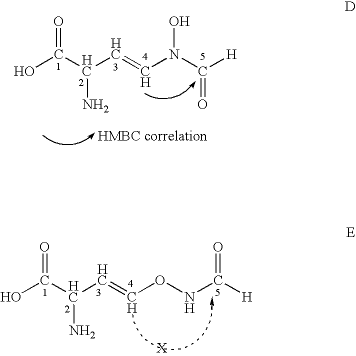

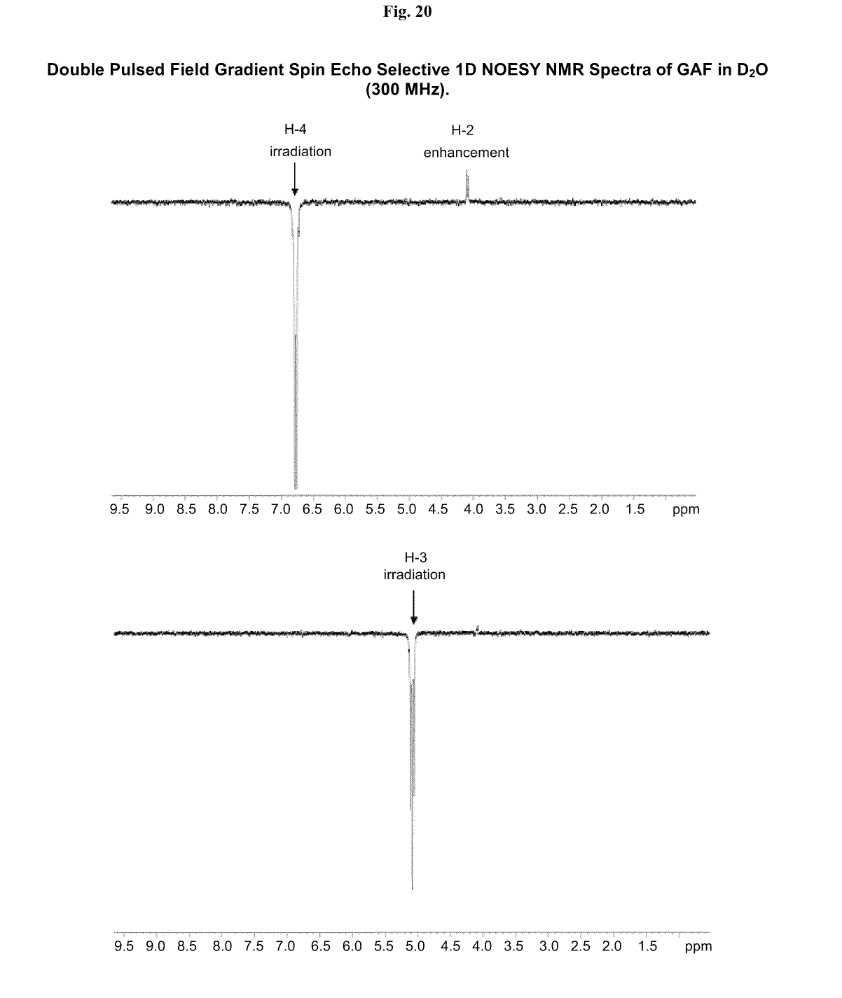

Structures D and E illustrated above, are alternate structures for GAF, in particular, D represents N-hydroxyformyl vinylglycine and E represents N-oxyformyl vinylglycine. A trans geometry of the olefin at C-3 was assigned on the basis of a selective nOe response between H-4 and H-2 (FIG. 20, spectrum A). In addition, neither a 2D NOESY correlation between H-3 and H-4, nor a reciprocal enhancement of H-3 upon selective excitation of H-4 in a 1D double pulsed field gradient spin echo NOESY experiment (FIG. 20, spectrum B) was observed as would be expected for a cis olefin. The planar structure 2 was therefore assigned to GAF: 2-amino-4-formylaminooxy-but-3E-enoic acid. Although it is most likely that the naturally-occurring GAF is an L-vinylglycine, the R/S configuration at the alpha carbon (C-2) has yet to be confirmed.

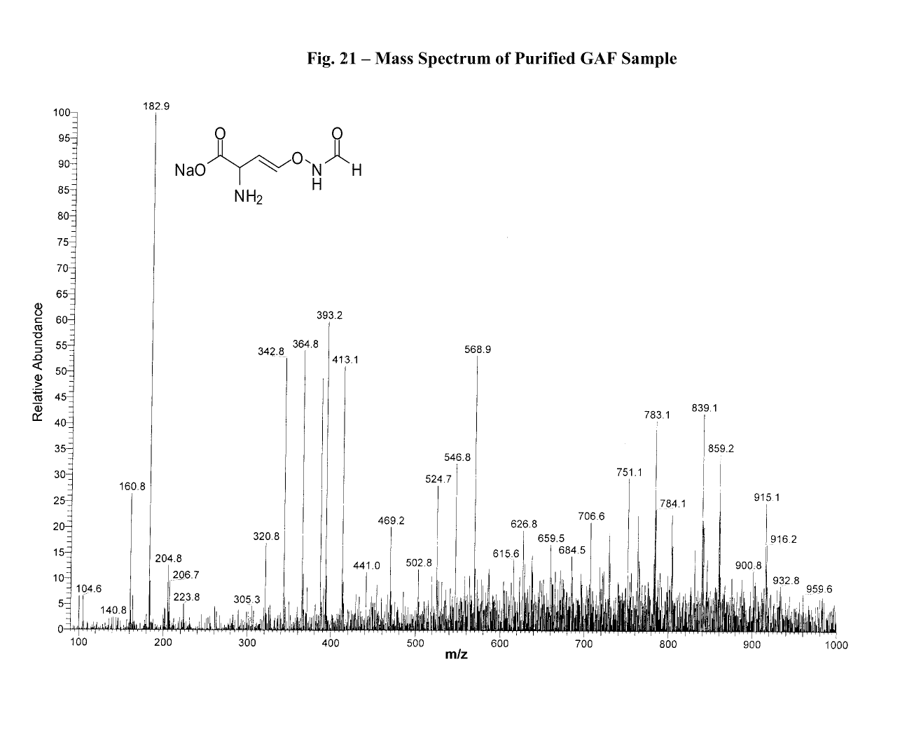

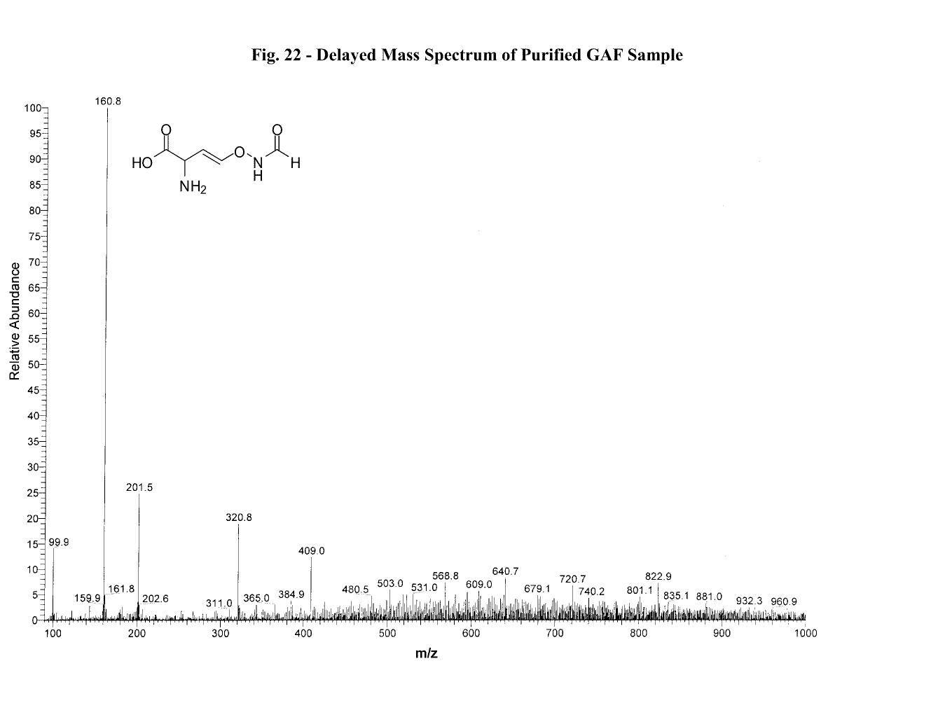

Mass spectrometric analysis was performed on the purified GAF sample, and the spectrum as obtained using direct injection electrospray ionization, is provided in FIG. 21. The most prominent peak seen in the mass spectrum was at 183 m/z. This peak was accompanied by smaller peaks on either side at 161 and 205 m/z respectively, suggesting a sodium adduct sequence based on a molecular species with a neutral molecular weight of 160. When the mass spectrometric scans were a little delayed in time, the resulting mass spectrum showed a single prominent peak at 161 m/z, suggesting that this molecular species was somewhat retarded in passing through the quartz injection tube (FIG. 22).

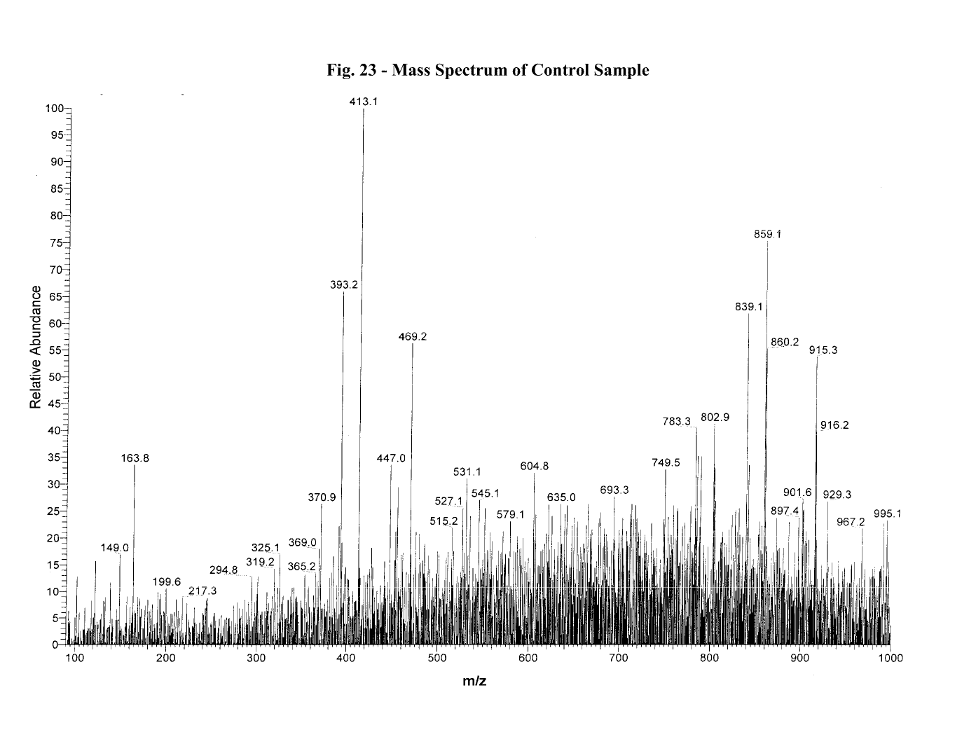

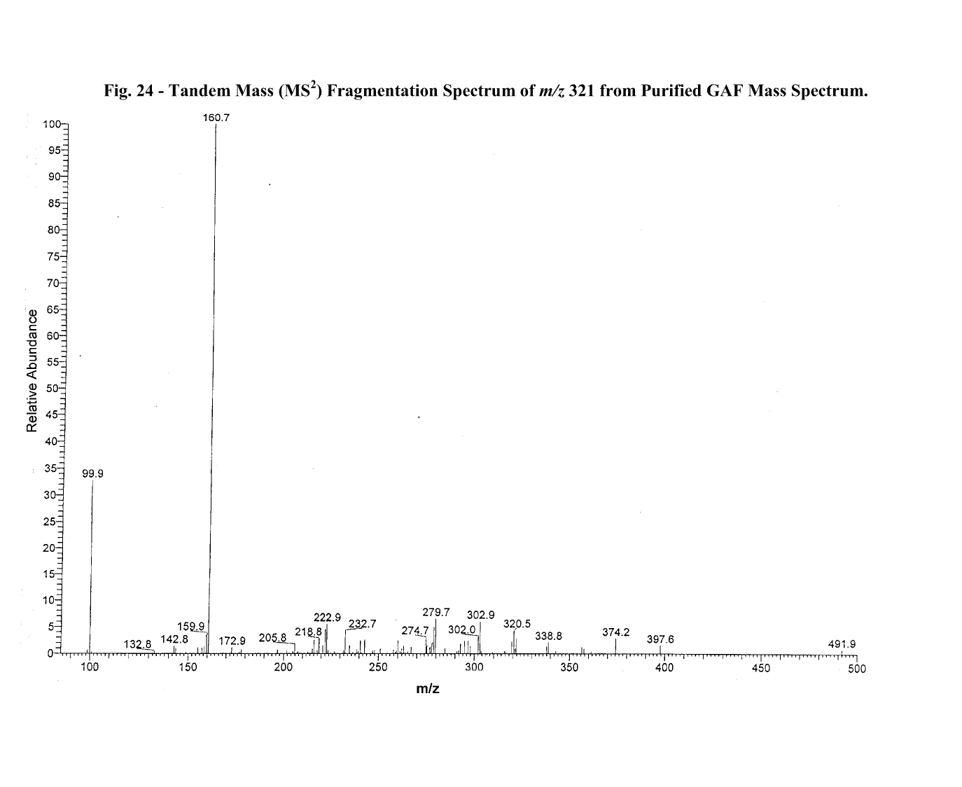

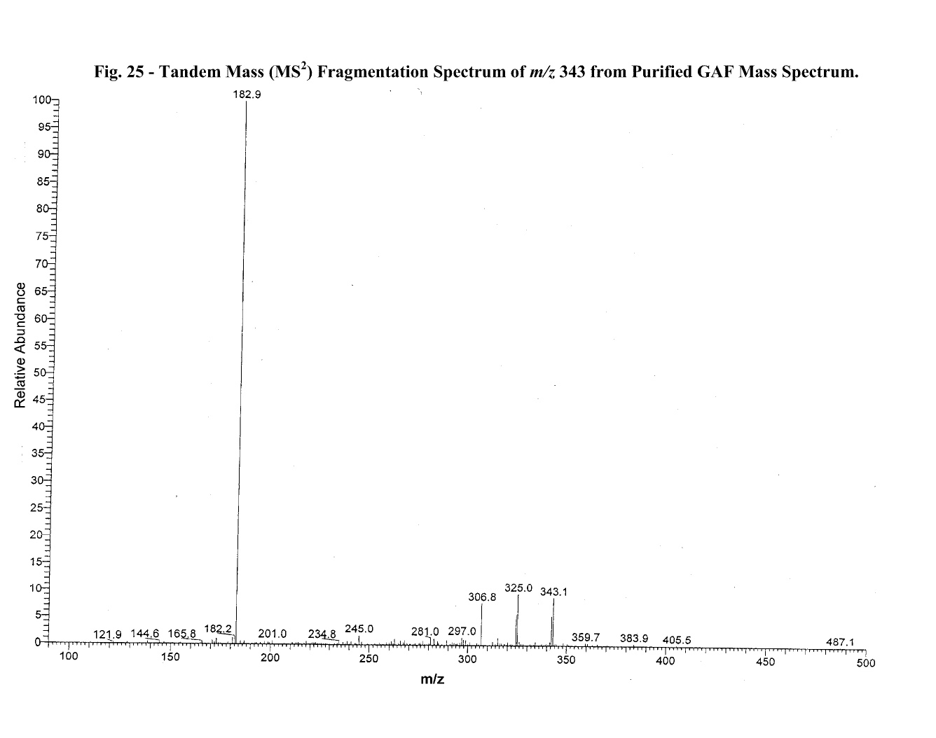

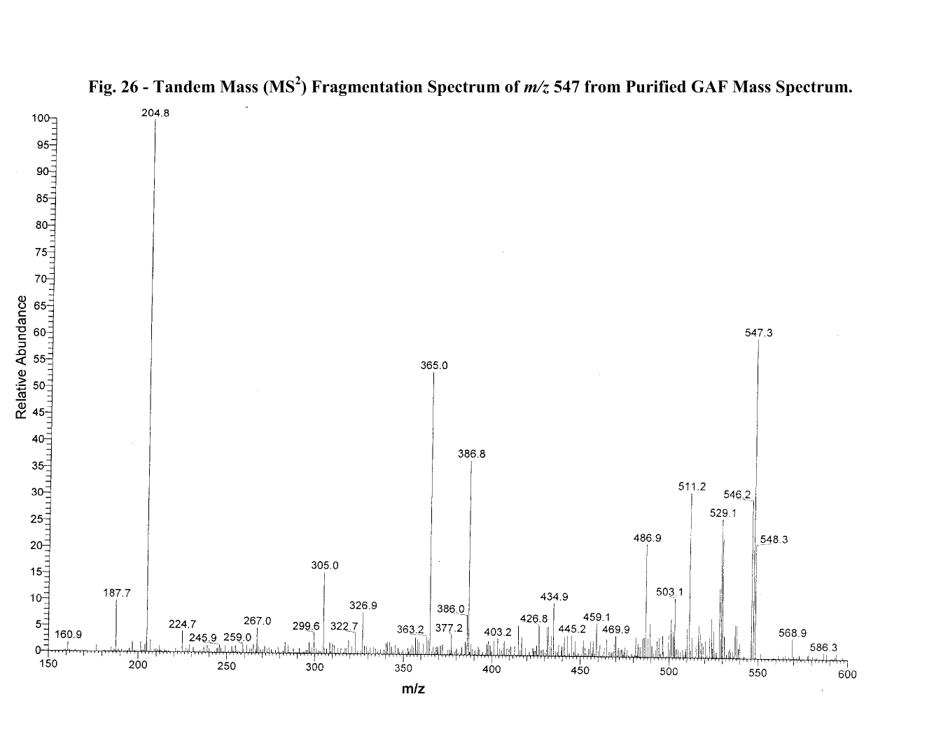

Although the mass spectrum of the purified GAF sample exhibited several other rather prominent peaks at higher m/z values, a number of these (including a cluster at 393, 413, 469 and clusters above 700 m/z) were also present in the mass spectrum of a control sample consisting of a similar volume of Sephadex® G15 column eluent taken from a position in the elution profile following the GAF peak. The mass spectrum of this sample is provided in FIG. 23. Another two clusters of triplets (321, 343, 365 and 525, 547, 569) appeared likely to be aggregates representing sodium adduct sequences of various combinations of the base 161,183, and 205 molecular species. This conclusion was based in part on the fact that MSⁿ fragmentation spectra of these species yielded 161, 183, or 205 as prominent m/z ions (See FIGS. 24, 25 and 26, respectively).

In view of the mass spectrometric analysis summarized above, GAF has a molecular weight of 160 Da and contains either zero or an even number of nitrogen (N) atoms. The fact that GAF gives a positive ninhydrin reaction test indicates that it includes at least one amino group. Given the apparent low molecular weight of 160, it appeared most likely that GAF contained 2 nitrogen atoms. Although the presence of 4 nitrogens could not be excluded, the resulting structure would be rather unusual.

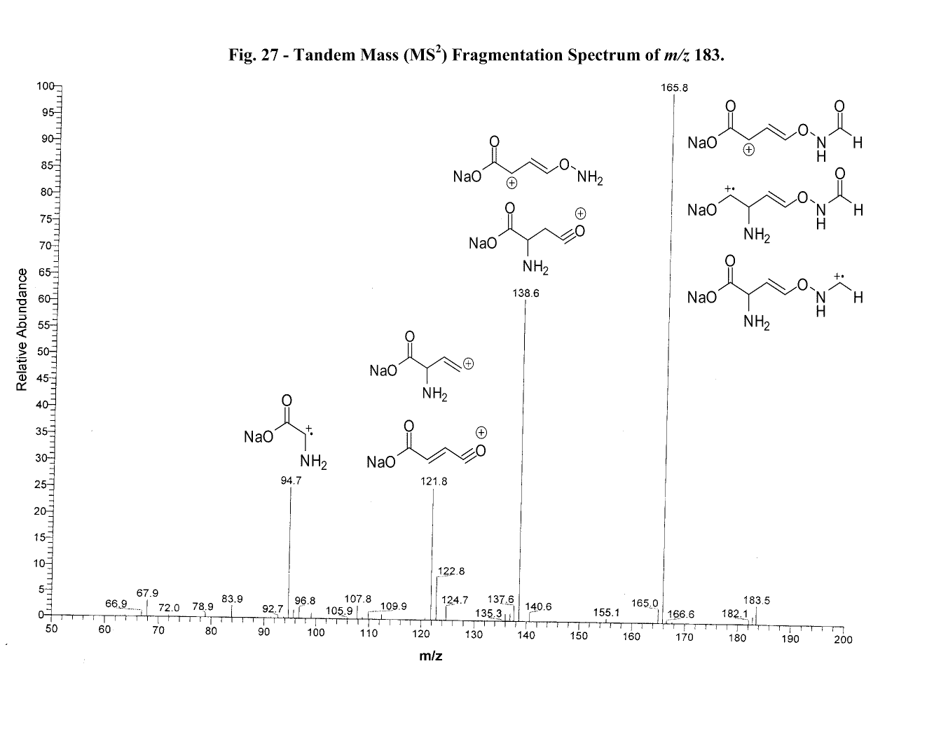

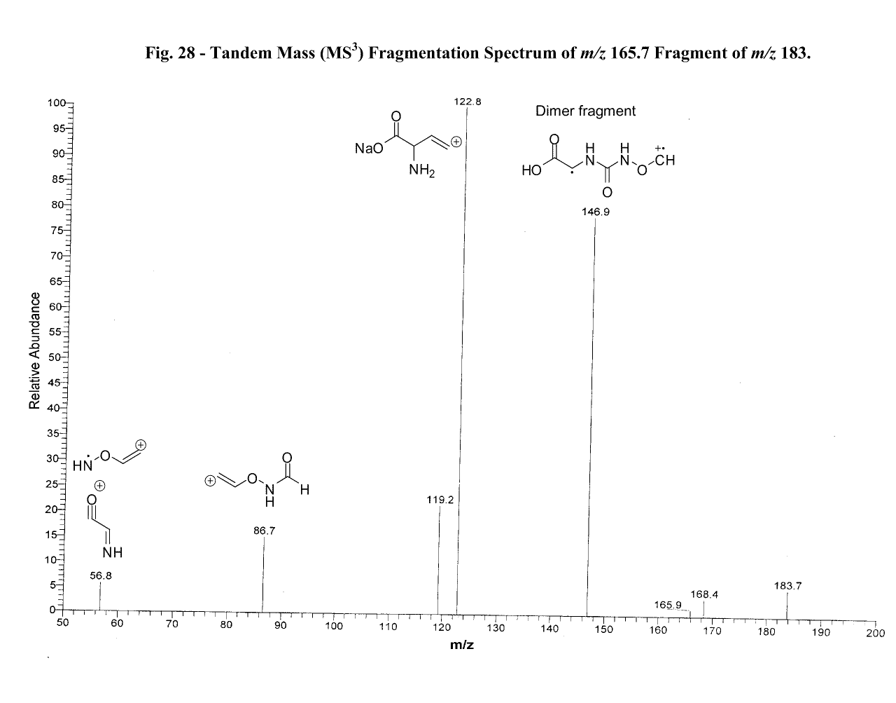

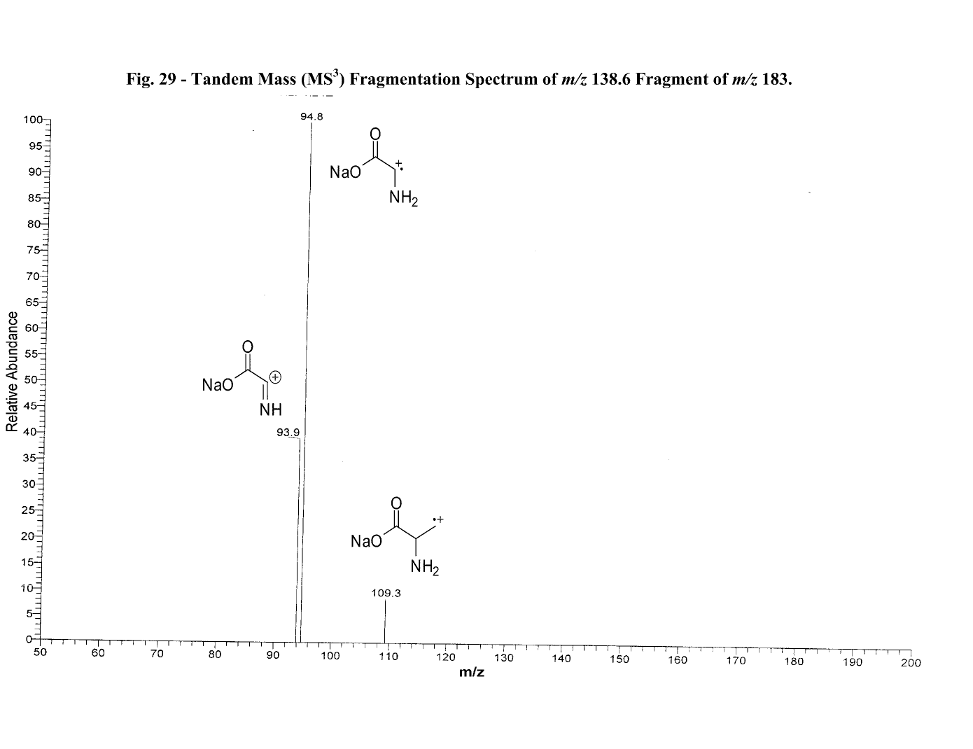

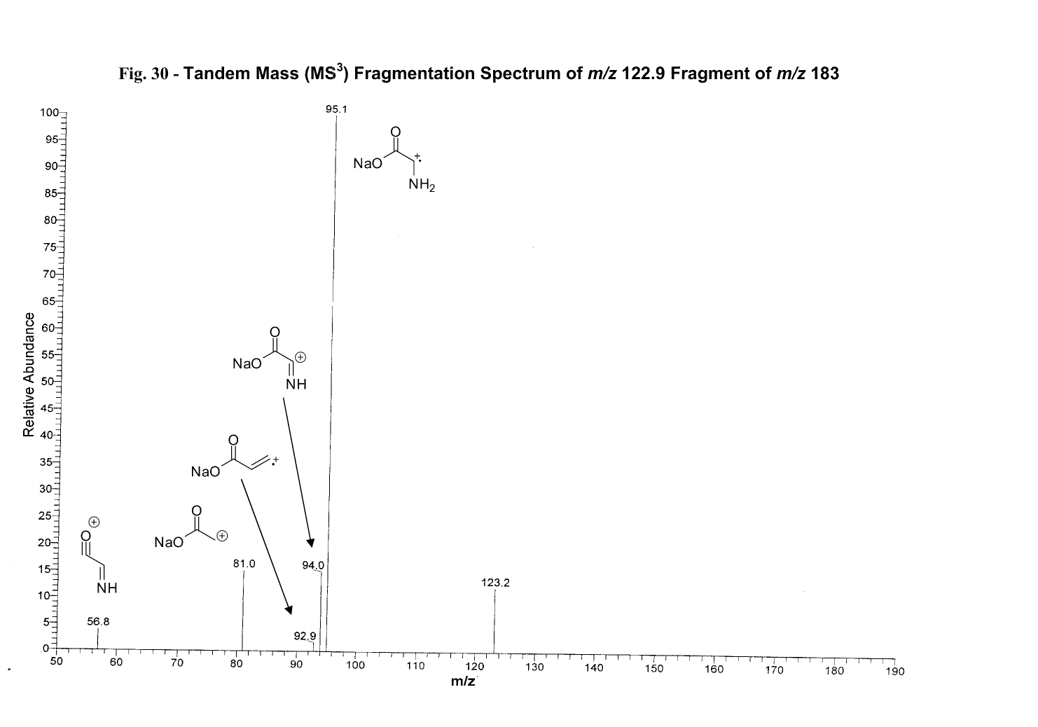

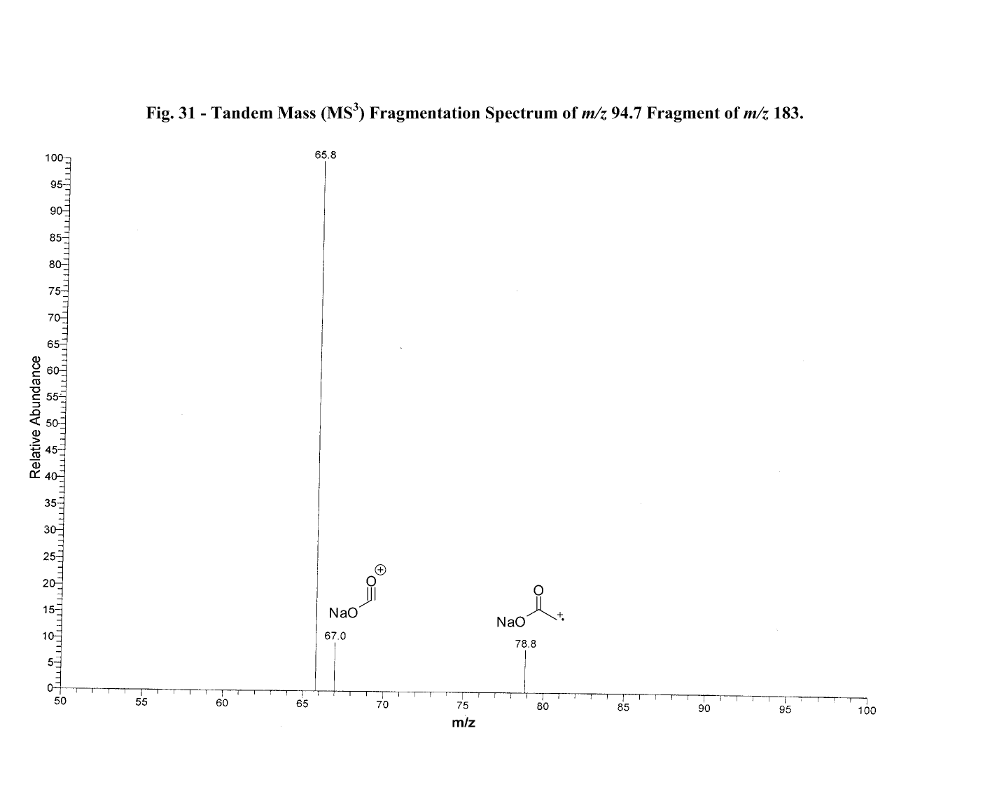

The fragmentation patterns obtained from performing MSⁿ tandem mass spectrometry on the fragment m/z 183 are shown in FIGS. 27 through 31. These fragmentations are made complex by the numerous labile functional groups in GAF which may present several possible patterns for each fragmentation. However, exemplary ion species are indicated in each FIG. for selected ions. Note that, although localized positive charges are indicated on the given fragments, these could also be represented as delocalized charges over the entire molecular ion species to encompass possible resonance structures. The use of localized charges is simply useful for rationalizing fragmentation processes. In summary, the mass spectrometric data are consistent with the empirical formula and structure deduced from the NMR data.

### Example 3

### Germination-Arrest Activity of Aminoethoxyvinylglycine (AVG) and AminoOxyacetic Acid (AOA) in the Poa Bioassay and in Soil-Based Systems

Identification of GAF as 2-amino-4-formylaminooxy-but-3-enoic acid (i.e., as 4-formylaminooxyvinylglycine) indicated that other vinylglycine compounds may have similar activity in arresting the germination of grassy weeds. Moreover, because vinylglycines are known to inhibit pyridoxal phosphate dependent enzyme reactions, it appeared likely that compounds of different structure but a similar mode of action should also be effective in arresting the germination of grassy weeds.

The compound 2-amino-4-aminoexthoxyvinylglycine (AVG, 2-amino-4-aminoethoxy-but-3-enoic acid) is available commercially, both as the pure compound and as a 15% formulation marketed under the trade name ReTain®. (ReTain® is used as a plant growth regulator to prevent blossom drop in orchard crops.) Thus, the availability of AVG made it possible to use this compound to test for the general ability of vinylglycines to arrest the germination of grassy weeds. Similarly, aminooxyacetic acid is a commercially available compound that is structurally distinct from vinylglycines but shares with vinylglycines the ability to inhibit pyridoxal phosphate dependent enzyme reactions. Therefore, both of these compounds were tested in the Standard Poa Bioassay System (described in Example 1) to determine whether they exhibited biological activity similar to that of GAF. The results of these bioassays are shown in Table 4. Both compounds exhibited GAF-like activity and arrested the germination of Poa annua seeds under these test conditions.

TABLE 4

| | GERMINATION SCORE IN THE POA BIOASSAY | | | | | | | |
|---|---|---|---|---|---|---|---|---|
| | AT THE INDICATED TEST CONCENTRATION | | | | | | | |
| | (±Standard Error of the Mean) | | | | | | | |
| COMPOUND | CONCENTRATION TESTED (mM) | | | | | | | |
| TESTED | 0 | 0.03 | 0.1 | 0.3 | 1.0 | 3.0 | 10.0 | 30.0 |
| AminoEthoxyVinylGlycine | 4.00 ± 0.00 | 2.3 ± 0.1 | 1.3 ± 0.1 | 1.0 ± 0.0 | 1.0 ± 0.0 | 1.0 ± 0.0 | 1.0 ± 0.0 | 0.9 ± 0.1 |
| (AVG) | | | | | | | | |
| AminoOxyAcetic Acid | 4.0 ± 0.0 | 2.5 ± 0.1 | 1.7 ± 0.1 | 1.2 ± 0.1 | 1.0 ± 0.0 | 1.0 ± 0.0 | 1.0 ± 0.0 | 0.9 ± 0.1 |
| (AOA) | | | | | | | | |

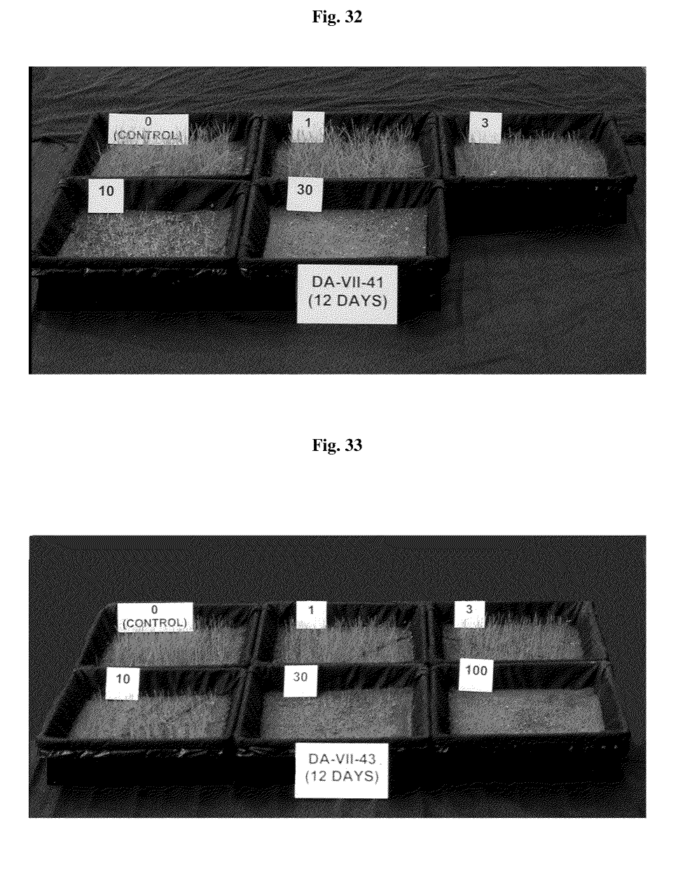

The effect of AVG on germination of seeds of Poa annua also was determined in a soil-based system using the protocol previously described. Briefly, annual bluegrass (Poa annua) seed was spread on the surface of soil filled flats and allowed to imbibe water for 48 hours. The imbibed seeds were sprayed with 50 mL of a solution consisting of ReTain® dissolved in 0.1% Tween® 20 to give the indicated mM concentrations of aminoethoxyvinylglycine (AVG). The seeds were sprayed again 24 hours later with the same volume of ReTain® solution. A photograph was taken 12 days after the first treatment (14 days after seeding of the flats). This photo is provided as a digital image in FIG. 32. As shown in this figure, AVG did arrest germination of Poa annua seeds under these test conditions.

The effect of AOA on the germination of Poa annua also was determined in a soil-based system. Briefly, annual bluegrass (Poa annua) seed was spread on the surface of soil filled flats and allowed to imbibe water for 48 hours. The imbibed seeds were sprayed with 50 mL of a solution of aminooxyAcetic Acid (AOA) dissolved in 0.1% Tween® 20 to give the indicated mM AOA concentrations. The seeds were sprayed again 24 hours later with the same volume of ReTain® solution. A photograph was taken 12 days after the first treatment (14 days after seeding of the flats). A digital image illustrating the effect of AOA is provided in FIG. 33. As shown in this figure, ΛOΛ also arrested germination of Poa annua seeds under these test conditions.

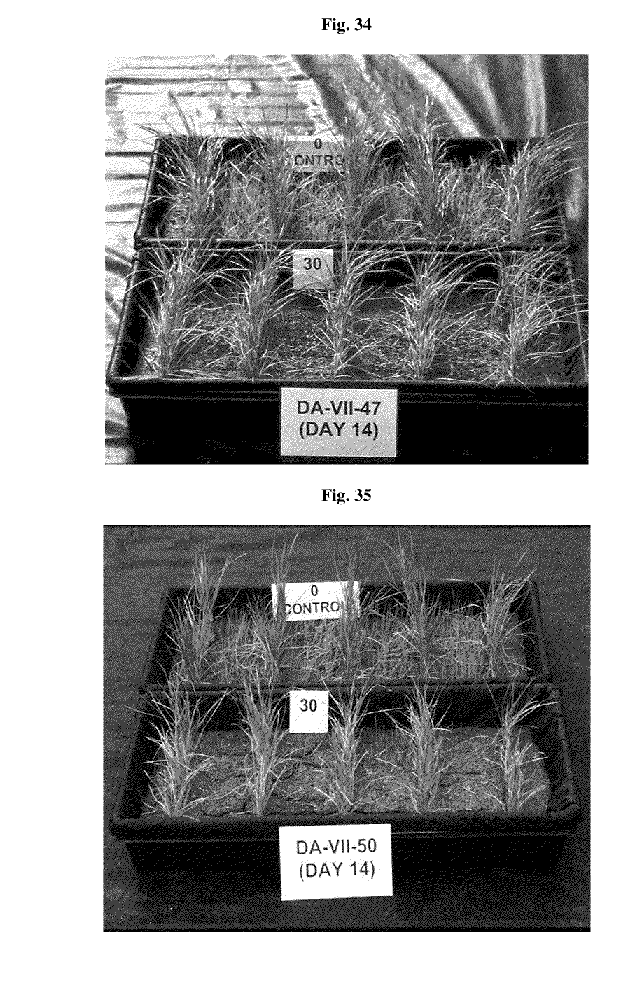

The ability of established perennial ryegrass (Lolium perenne) seedlings to tolerate AVG concentrations sufficient to arrest the germination of Poa annua seeds was also tested in soil-based systems. Perennial ryegrass (Lolium perenne) seedlings were grown in rows in soil-filled flats to 21 days of age. At that time, the spaces between the ryegrass rows were seeded with annual bluegrass (Poa annua). The seeds were allowed to imbibe water for 48 hours and then sprayed with 50 mL of a solution consisting of ReTain® dissolved in 0.1% Tween® 20 to give the indicated mM concentrations of aminoethoxyvinylglycine (AVG). The seeds were sprayed again 24 hours later with the same volume of ReTain® solution. A photograph was taken 12 days after the first treatment (14 days after seeding of the flats) and a digital image demonstrating the effect of AVG is provided in FIG. 34. As shown in this figure, AVG applied at a concentration that completely arrested germination of Poa annua seeds had little if any effect on established perennial ryegrass seedlings.

The ability of established perennial ryegrass (Lolium perenne) seedlings to tolerate AOA concentrations sufficient to arrest the germination of Poa annua seeds was also tested in soil-based systems. As in the tests of AVG, perennial ryegrass (Lolium perenne) seedlings were grown in rows in soil-filled flats to 21 days of age. At that time, the spaces between the ryegrass rows were seeded with annual bluegrass (Poa annua). The seeds were allowed to imbibe water for 48 hours and then sprayed with 50 mL of a solution of aminooxyacetic acid (AOA) dissolved in 0.1% Tween 20 to give the indicated mM concentrations of AOA. The seeds were sprayed again 24 hours later with the same volume of AOA solution. A photograph was taken 12 days after the first treatment (14 days after seeding of the flats). The resulting digital image is provided in FIG. 35. In this case, although germination of seeds of Poa annua were arrested as expected, some damage was evident in the established ryegrass seedlings. However, it is possible that these seedlings could recover with sufficient time in the field.

### Example 4

### Amino Acid Reversal of the Effects of GAF on Germination of Poa annua

AVG and AOA are both known to act as inhibitors of pyridoxalphosphate dependent enzyme reactions. Enzymes that catalyze such reactions are known to include both a variety of aminotransferases (enzymes that are important in nitrogen metabolism) and 1-aminocyclopropane-1-carboxylate (ACC) synthase, a key enzyme in the biosynthesis of the plant hormone ethylene. To further test the hypothesis that germination-arrest activity can be expected to be associated with chemical agents that target such enzymes, the ability of exogenously supplied amino acids to reverse the effects of the Germination Arrest Factor present in P. fluorescens Strain WH6 culture filtrate was tested in the Poa bioassay. Table 5 summarizes the data for those amino acids that significantly reversed the effects of GAF under these test conditions (significant reversal is defined as the recovery of germination scores of 3.0 to 4.0 in the presence of the specified concentration of WH6 culture filtrate).

TABLE 5
Interactions of GAF with Amino Acids that Significantly Reversed Germination
Arrest Activity in the Poa Bioassay.

| | WH6 | | | | | | |
|---|---|---|---|---|---|---|---|
| | CULTURE | | | | | | |
| | FILTRATE | GERMINATION SCORE | | | | | |
| | Concentration | (±Standard Error of the Mean) | | | | | |
| AMINO | (1X = No | At Indicated Amino Acid concentration (mM) | | | | | |
| ACID | Dilution) | 0 | 0.3 | 1 | 3 | 10 | 30 |
| Alanine | 0 | 3.8 ± 0.1 | 4.0 ± 0.0 | 3.9 ± 0.1 | 4.0 ± 0.0 | 3.9 ± 0.1 | 3.2 ± 0.1 |
| | 0.1X | 1.3 ± 0.1 | 1.9 ± 0.1 | 3.3 ± 0.1 | 4.0 ± 0.0 | 4.0 ± 0.0 | 4.0 ± 0.0 |
| Glutamine | 0 | 4.0 ± 0.0 | 4.0 ± 0.0 | 4.0 ± 0.0 | 3.7 ± 0.3 | 3.7 ± 0.3 | 4.0 ± 0.0 |
| | 0.1X | 1.3 ± 0.1 | 1.4 ± 0.1 | 1.8 ± 0.2 | 3.5 ± 0.3 | 4.0 ± 0.0 | 4.0 ± 0.0 |
| Leucine | 0 | 4.0 ± 0.0 | 3.9 ± 0.2 | 4.0 ± 0.0 | 3.6 ± 0.2 | 3.0 ± 0.0 | 1.7 ± 0.1 |
| | 0.1X | 1.0 ± 0.0 | 1.5 ± 0.0 | 1.5 ± 0.3 | 3.6 ± 0.1 | 3.4 ± 0.1 | 2.8 ± 0.1 |

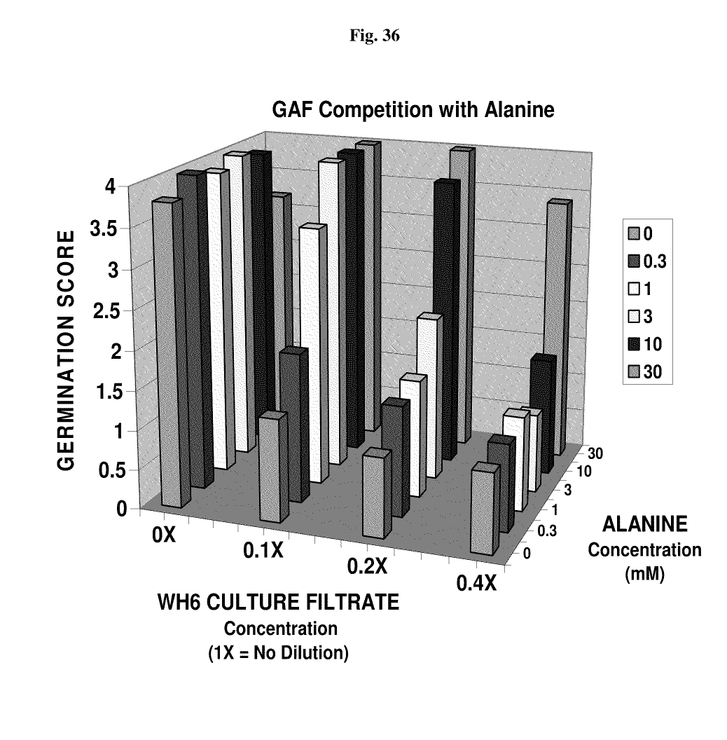

A Germination Score of 1.0 indicates high GAF activity (germination completely arrested immediately after emergence of the coleorhiza and plumule). A Germination Score of 4.0 indicates no GAF activity (germination and seedling development equivalent to that of controls). Among the amino acids tested, glutamine and alanine were the most effective. Glutamine is known to play a central role in nitrogen assimilation in plants, and alanine is known to play a key role in nitrogen metabolism under hypoxic conditions such as that occur in the fluid-filled wells used in the Poa bioassay or in the frequently water-saturated fields associated with grass seed production systems (Good & Crosby, Plant Physiol. 90: 1305-1309, 1989). A more detailed examination of the ability of alanine to interact with and reverse the action of GAF in arresting the germination of Poa seeds is illustrated in FIG. 36 and has also been presented in Armstrong et al., Biological Control, doi:10.1016/j.biocontrol.2009.06.004, 2009. The ability of these amino acids to reverse the effects of GAF in arresting the germination of Poa seeds is consistent with an effect of this compound on amino acid metabolism through an ability to interfere with pyridoxal phosphate dependent enzyme reactions.

### Example 5

### Molecular Correlation between Grassy Weed Inhibition and GAF-Specific Genotype in Pseudomonas Isolates

This example describes that inhibition of grassy weeds is associated with the presence of a GAF-specific formyl-transferase gene in various Pseudomonas strain isolates. Bacteria were cultured, and culture filtrate prepared, analyzed by TLC, and used in the Poa bioassay as described in Example 1. GAF-associated grassy weed inhibition in various Pseudomonas isolates directly correlated with the presence the ninhydrin-staining band in TLC analysis of culture filtrates, as well as the presence of the formyl-transferase gene in GAF-producing isolates. Thus, the presence of the GAF-specific formyl-transferase gene in a given bacterium is predictive of GAF activity.

### Identification of the Pseudomonas Isolates and Strains Tested for GAF-Production.

The origin and data used to identify the various Pseudomonas rhizosphere bacteria that we have isolated and tested for GAF production are summarized in Table 6. The five isolates labeled Group 1 are the five isolates previously described in U.S. application Ser. No. 10/537,017, U.S. application Ser. No. 12/077,648, and International Patent Publication WO 04/052097. The seven isolates labeled Group II have also been obtained from rhizosphere sources, as indicated, and represent additional isolates. Table 7 lists five strains of P. fluorescens that were obtained from other laboratories for purposes of comparison with our isolates. This group includes two P. fluorescens strains (Pf5 and PfO-1) with genomes that have been completely sequenced. GenBank accession numbers are provided for these two strains. A yet to be identified strain of Pseudomonas was included in this group because it was readily available.

Taxonomic identification of the twelve isolates that constitute Groups I and II is based on both fatty acid methyl-ester analysis (FAME analysis) and DNA sequence analysis of a 400+ base pair region near the beginning of their respective 16S-rRNA genes. Based on the DNA sequence information, all twelve of our isolates appear to be variants of Pseudomonas fluorescens. FAME analysis yielded similar identifications of eleven of these isolates. However, one (AD31) in Group I was identified by FAME analysis as an isolate of P. putida. Such taxonomic ambiguity is not unusual in bacterial taxonomy. Our tendency is to rely on the rDNA sequencing information and regard AD31 as a somewhat unusual variant of P. fluorescens.

TABLE 6
Origin and Taxonomic Identification of Selected Deleterious Rhizosphere Bacteria
(DRB) Isolates.

| | | RHIZOSPHERE | TAXONOMIC IDENTIFICATION | |
|---|---|---|---|---|
| | SITE OF | SOURCE | FAME | rDNA |
| ISOLATE | ISOLATION | OF ISOLATE | ANALYSIS | SEQUENCING |
| GROUP I | | | | |
| AD31 | Cut Bank, Alsea Valley | Poa Species | Pseudomonas putida | Pseudomonas |
| | Benton County, OR | | Biotype B/vancouverensis | fluorescens |
| AH4 | Disturbed Site, Alsea | Poa Species | Pseudomonas fluorescens | Pseudomonas |
| | Valley | | Biotype A | fluorescens |
| | Benton County, OR | | | |
| E34 | Hyslop Research Farm, | Poa Species | Pseudomonas fluorescens | Pseudomonas |
| | OSU | | Biotype G/taetrolens | fluorescens |
| | Benton County, OR | | | |
| WH6 | Hyslop Research Farm, | Wheat Cultivar | Pseudomonas fluorescens | Pseudomonas |
| | OSU | | Biotype G/taetrolens | fluorescens |
| | Benton County, OR | | | |
| WH19 | Hyslop Research Farm, | Wheat Cultivar | Pseudomonas fluorescens | Pseudomonas |
| | OSU | | Biotype A | fluorescens |
| | Benton County, OR | | | |
| GROUP II | | | | |
| AH10 | Disturbed Site, Alsea | Poa Species | Pseudomonas fluorescens | Pseudomonas |
| | Valley | | Biotype G/taetrolens | fluorescens |
| | Benton County, OR | | | |
| BT1 | Botany Research Farm, | Wheat Cultivar | Pseudomonas fluorescens | Pseudomonas |
| | OSU | | Biotype A | fluorescens |
| | Linn County, OR | | | |
| E24 | Hyslop Research Farm, | Poa Species | Pseudomonas fluorescens | Pseudomonas |
| | OSU | | Biotype B | fluorescens |
| | Benton County, OR | | | |
| TDH5 | Private Vegetable Farm | Poa Species | Pseudomonas fluorescens | Pseudomonas |
| | Benton County, OR | | Biotype B | fluorescens |
| TR33 | Botany Research Farm, | Triticale Species | Pseudomonas fluorescens | Pseudomonas |
| | OSU | | Biotype A | fluorescens |
| | Linn County, OR | | | |
| TR44 | Botany Research Farm, | Triticale Species | Pseudomonas fluorescens | Pseudomonas |
| | OSU | | Biotype A | fluorescens |
| | Linn County, OR | | | |
| TR46 | Botany Research Farm, | Triticale Species | Pseudomonas fluorescens | Pseudomonas |
| | OSU | | Biotype G/taetrolens | fluorescens |
| | Linn County, OR | | | |

Fatty acid methyl ester analyses (FAME analyses) and DNA sequencing of a 400+ by region of the 16S-rRNA gene were performed by Microcheck, Inc. (Northfield, Vt.), and the reported taxonomic identities are based on Microcheck data bases. Group I isolates were described in U.S. patent application Ser. No. 10/537,017. Group II isolates are characterized herein.

TABLE 7
Strains of Pseudomonas fluorescens Obtained from Other Laboratories.

| | SOURCE | |
|---|---|---|
| PSEUDOMONAS | FROM WHICH STRAIN WAS | |
| STRAIN | OBTAINED | FEATURES OF INTEREST |
| Pseudomonas | Dr. Joyce Loper | Genome completely sequenced. |
| fluorescens Pf5 | USDA/ARS Horticultural Crops | GenBank Accession # |
| | Research Laboratory, Corvallis, | NC_004129 |
| | OR | |
| Pseudomonas | Dr. Mark Silby | Genome completely sequenced. |
| fluorescens PfO-1 | Dept. Mol. Biology & | GenBank Accession # CP000094 |
| | Microbiology | |
| | Tufts University School of | |
| | Medicine, | |
| | Boston, MA | |
| Pseudomonas | Dr. Joyce Loper | Strain used in Loper laboratory |
| fluorescens A506 | USDA/ARS Horticultural Crops | for various molecular studies. |
| | Research Laboratory, Corvallis, | |
| | OR | |
| Pseudomonas Sp. A17 | Dr. Lloyd Elliott | Uncharacterized Pseudomonas |
| | USDA/ARS National Forage Seed | isolate obtained from Oregon |
| | Production Research Center, | soils at unrecorded site. |
| | Corvallis, OR | |
| Pseudomonas | Dr. Ann Kennedy | Pseudomonas strain reported to |
| fluorescens D7 | USDA/ARS | cause stunting of grassy weeds. |
| | Washington State University | One of strains cited by patent |
| | Pullman, WA | examiner as possible prior art. |

Diversity in the 400+ by rDNA Sequence used for Taxonomic Identification of Pseudomonas Isolates.

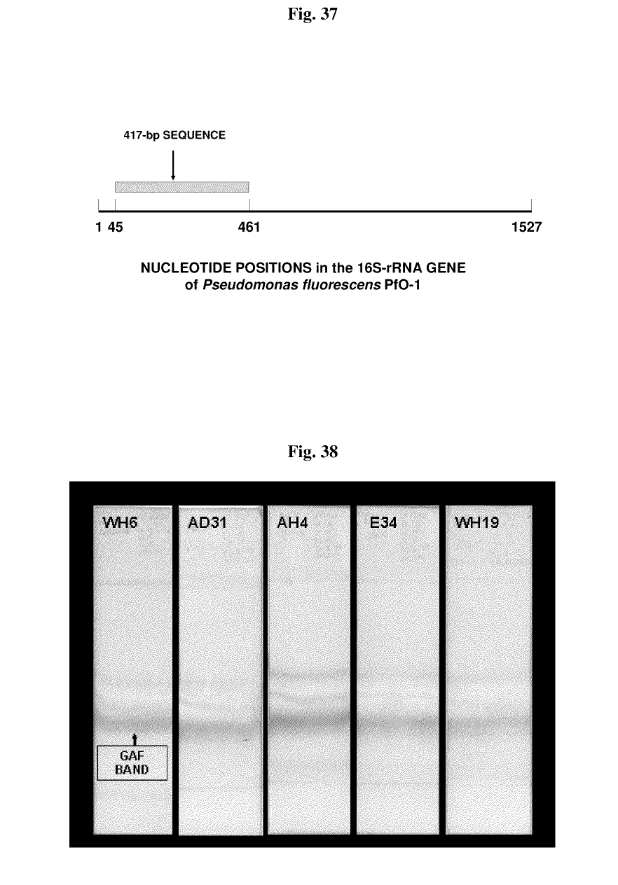

The 400+ by rDNA sequence used for taxonomic classification of our Pseudomonas isolates corresponded to a region early in the 16S-rRNA gene. The alignment of a 417-bp portion of this sequence relative to the complete 16S-rDNA sequence from P. fluorescens PO-1 (for which the complete genome sequence is available) is shown schematically in FIG. 37 (This 417-bp sequence was selected for further analysis because both forward and reverse sequence data were obtained for this segment). The actual base sequence of the corresponding 417-bp segment from the P. fluorescens Isolate WH6 is set forth as SEQ ID NO: 1. The variations within the corresponding 417-bp sequences in the other Pseudomonas isolates and strains considered here are summarized in Table 8. This sequence is identical in all of the isolates that constitute Group I (the five isolates described in the referenced patent application) and in one of the isolates (TR44) in Group II. Among all bacterial lines tested, the largest number of deviation from the WH6 sequence was found in P. fluorescens Strain Pf5, which contained two insertions and 33 base substitutions relative to the 417-bp sequence from WH6. Among the isolates tested, the most diversity was found in Isolate E24, where the corresponding 417-bp sequence exhibited 19 single-base substitutions and two single-base insertions.

One may conclude from these data that the P. fluorescens isolates and strains analyzed here represent a range of intra-specific genetic diversity. Nevertheless, the five isolates described are clearly closely related, and the data discussed below establish that all GAF-producing Pseudomonas bacteria share certain distinguishing features.

TABLE 8
Diversity in a 417-BP Sequence from the 16S-rRNA Gene of Various
Pseudomonas Isolates and Strains.

| | SEQUENCE CHANGES | | |
|---|---|---|---|
| | RELATIVE TO A 417-BP 16S-rDNA SEQUENCE | | |
| | FROM P. FLUORESCENS ISOLATE WH6 | | |
| | | SINGLE | INSERTIONS |
| ISOLATE | SINGLE BASE | BASE | (Number of |
| OR STRAIN | SUBSTITUTIONS | DELETIONS | Bases) |
| GROUP I | | | |
| AD31 | None | None | None |
| AH4 | None | None | None |
| E34 | None | None | None |
| WH19 | None | None | None |
| GROUP II | | | |
| AH10 | 1 | None | None |
| BT1 | 1 | None | None |
| E24 | 19 | None | 2 (Single Base) |
| TDH5 | 2 | None | None |
| TR33 | 1 | None | None |
| TR44 | None | None | None |
| TR46 | 4 | 2 | None |
| STRAINS | | | |
| Pf5 | 33 | None | 2 (Single Base) |
| | | | 1 (Eight Base) |
| PfO-1 | 10 | None | None |
| A506 | Unknown | Unknown | Unknown |
| A17 | Unknown | Unknown | Unknown |
| D7 | Unknown | Unknown | Unknown |

The nucleotide sequence of a 417-bp region near the start of the 16S-rRNA gene from P. fluorescens WH6 (SEQ ID NO: 1) was aligned and compared with the analogous sequences from the other Pseudomonas isolates and strains tested here. (This particular sequence was selected for comparison because both forward and reverse sequence data were available in this region of the gene.) The source of the sequence data analyzed in this manner was the sequence information provided by Microcheck, Inc. as described in Example 1.

Biological Activity of Culture Filtrates from Pseudomonas Isolates and Strains

Culture filtrates from all of the Pseudomonas isolates and strains listed in Tables 6 and 7 were prepared and tested for Germination-Arrest Factor Activity (GAF Activity) in the Standard Poa Bioassay System described in Example 1. In this bioassay protocol, seeds of Poa annua (annual bluegrass, ABG) are used as the standard test material, and germination of these seeds is scored according to the criteria listed therein. In this scoring system, a score of 1.0 represents complete germination-arrest at a developmental stage immediately after the plumule and coleorhiza have emerged from the seed coat. A score of 4.0 represents essentially normal germination under the specified assay conditions, with germination having proceeded to where the first true leaf has substantially emerged from the coleoptile and is green in color, and the primary root is visible and elongated.

The GAF activity recovered in the culture filtrates prepared from the various Pseudomonas isolates and strains is shown in Table 9. As expected, all of the Isolates in Group I exhibited high GAF activity (bioassays scores of 1.0 at even the 0.3× dilutions). Similar results were obtained with five of the seven Group II isolates, but Isolate BT1 exhibited relatively weak activity in the bioassay, and Isolate TDH5 had very little, if any, GAF activity, bringing about only a slight stunting of normal seedling growth when full-strength culture filtrate was tested. The Pseudomonas strains obtained from other laboratories also exhibited little evidence of GAF activity, although, as in the case of TDH5, full-strength culture filtrates of some strains produced a slight slowing of normal seedling development.

In summary, these results demonstrate that the production of GAF activity is not a uniform trait associated with all Pseudomonas fluorescens isolates and strains. Moreover, the Poa bioassay serves to distinguish between GAF-producing Pseudomonas bacteria and Pseudomonas strains such as P. fluorescens D7. Although D7 does cause some stunting of P. annua seedlings, culture filtrates from this organism do not cause the germination-arrest observed with WH6 culture filtrates.

TABLE 9
Biological Activity of Culture Filtrates from Various Pseudomonas
Isolates and Strains as Tested in the Standard Poa Bioassay.

| | MEAN GERMINATION SCORE IN THE | | |
|---|---|---|---|
| | POA BIOASSAY AT THE | | |
| | INDICATED CULTURE FILTRATE DILUTION | | |
| SOURCE OF | (±Standard Error of the Mean) | | |
| CULTURE | | | 1.0X |
| FILTRATE | 0.1X | 0.3X | (Full-Strength) |
| CONTROL | | | |
| (PMS | 4.0 ± 0.00 | 4.0 ± 0.00 | 4.0 ± 0.00 |
| Medium) | | | |
| GROUP I | | | |
| AD31 | 1.9 ± 0.52 | 1.0 ± 0.00 | 1.0 ± 0.00 |
| AH4 | 0.9 ± 0.03 | 1.0 ± 0.00 | 1.0 ± 0.00 |
| E34 | 1.0 ± 0.00 | 1.0 ± 0.00 | 1.0 ± 0.00 |
| WH6 | 1.0 ± 0.00 | 1.0 ± 0.00 | 1.0 ± 0.00 |
| WH19 | 1.2 ± 0.17 | 1.0 ± 0.03 | 1.0 ± 0.00 |
| GROUP II | | | |
| AH10 | 1.0 ± 0.00 | 1.0 ± 0.00 | 1.0 ± 0.00 |
| BT1 | 1.7 ± 0.12 | 1.5 ± 0.00 | 1.0 ± 0.00 |
| E24 | 1.0 ± 0.00 | 1.0 ± 0.00 | 1.0 ± 0.00 |
| TDH5 | 4.0 ± 0.00 | 4.0 ± 0.00 | 2.8 ± 0.15 |
| TR33 | 1.0 ± 0.00 | 1.0 ± 0.00 | 1.0 ± 0.00 |
| TR44 | 1.0 ± 0.00 | 1.0 ± 0.00 | 1.0 ± 0.00 |
| TR46 | 1.0 ± 0.00 | 1.0 ± 0.00 | 1.0 ± 0.00 |
| STRAIN | | | |
| Pf5 | 4.0 ± 0.00 | 4.0 ± 0.00 | 4.0 ± 0.00 |
| PfO-1 | 4.0 ± 0.00 | 4.0 ± 0.00 | 3.4 ± 0.13 |
| A506 | 4.0 ± 0.00 | 4.0 ± 0.00 | 3.0 ± 0.15 |
| A17 | 4.0 ± 0.00 | 3.9 ± 0.05 | 3.0 ± 0.14 |
| D7 | 4.0 ± 0.00 | 4.0 ± 0.00 | 3.0 ± 0.19 |

Growth of bacterial cultures, preparations of culture filtrates, and Standard Poa Bioassay procedures are described in Example 1. Germination Scores were assigned according to the system summarized therein. A score of 4.0 represents normal germination and seedling development under the proscribed test conditions, and a score of 1.0 represents germination arrest immediately after initiation of germination.

### Thin-Layer Chromatographic (TLC) Analysis of Culture Filtrates

Data in U.S. application Ser. No. 10/537,017 demonstrated that the GAF activity present in culture filtrates of P. fluorescens isolate WH6 was associated with a particular ninhydrin-reactive band that could be seen on TLC plates used to fractionate extracts of WH6 culture filtrates. As described therein, mutational analysis of isolate WH6 confirmed that this ninhydrin-reactive compound was actually the Germination-Arrest Factor. Mutations of WH6 (brought about by transposon mutagenesis) that led to the loss of GAF-production also resulted in the disappearance of this particular ninhydrin-reactive band. If the wild-type phenotype was restored to the mutants by reintroducing the wild-type genes (cloned on plasmids) into the mutants, both GAF activity and the specific ninhydrin-reactive compound reappeared in the culture filtrate. As described in Example 2, the ninhydrin-reactive compound was isolated and identified as the Germination Arrest Factor (GAF, 2-amino-4-formylaminooxy-but-3-enoic acid) and its herbicidal activity was demonstrated (Table 2). Thus, the presence of this particular ninhydrin-reactive band in TLC chromatograms can be used as a distinguishing feature of culture filtrate of GAF-producing bacteria.

Ninhydrin-stained TLC chromatograms prepared from extracts of the culture filtrates of five Pseudomonas isolates (Group I Isolates of Table 6) are shown in FIG. 38. The culture filtrates of all five of these isolates caused germination-arrest in Poa annua seeds (Table 9), and an identical, ninhydrin-reactive band at the Rf of the Germination-Arrest Factor was recovered from the culture filtrates of all five isolates.

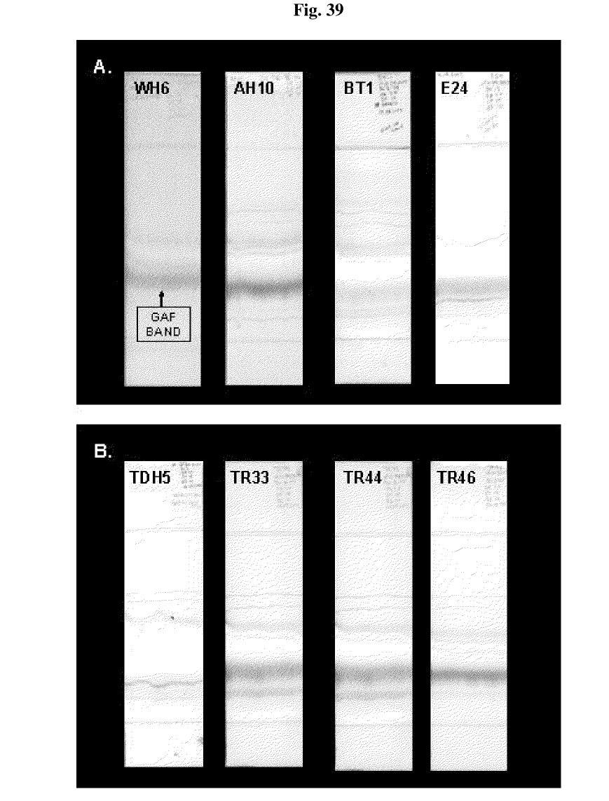

Similar ninhydrin-stained TLC chromatograms were prepared from the culture filtrates of the seven Group II isolates. As shown in FIG. 39A and B, culture filtrates from the five isolates that had strong germination-arrest activity (AH10, E24, TR33, TR44, and TR46, see Table 9) yielded ninhydrin-reactive bands at the expected Rf of GAF (based on the chromatogram for WH6). In contrast, this particular ninhydrin-reactive band is absent in the chromatogram prepared from TDH5 culture filtrate (the isolate that lacked GAF activity in the Poa bioassay shown in Table 9). Moreover, this band was much reduced in intensity in the chromatogram prepared from BT1 culture filtrate (the isolate that exhibited only weak GAF activity in the Poa bioassay shown in Table 9).

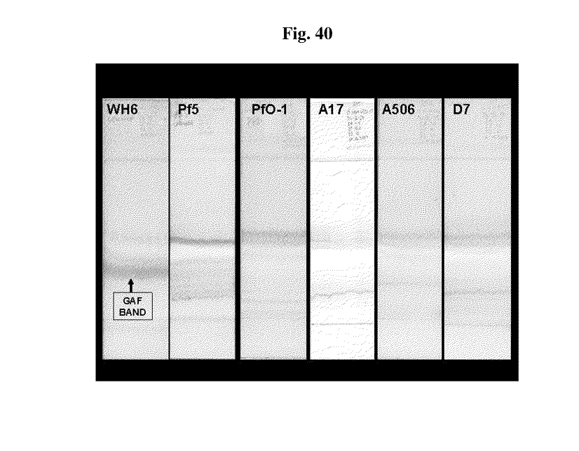

The corresponding TLC chromatograms prepared for culture filtrates of the five Pseudomonas strains acquired from other laboratories are shown in FIG. 40. None of these five strains exhibited germination-arrest activity in the Poa bioassay (Table 9), although some exerted a slight stunting or slowing of the development of Poa annua seedlings. As expected from the bioassay results, the GAF-specific ninhydrin band was absent from all five of these chromatograms, including the chromatogram from culture filtrates produced by P. fluorescens Strain D7.

In summary, the TLC data shown here further demonstrate that the same ninhydrin-reactive compound is produced by all Pseudomonas isolates and strains that possess GAF activity in the Poa bioassay. Moreover, those isolates and strains that fail to show germination-arrest activity in the Poa bioassay do not produce the specific ninhydrin-reactive compound that we show in Example 2 to be the GAF.

### Molecular Genetic Analysis of the Various Pseudomonas Isolates and Strains.

Data in U.S. application Ser. No. 10/537,017 included DNA sequences for various sites in the P. fluorescens WH6 genome that had been identified by mutational analysis to be genes involved in the production of enzymes or other proteins involved in GAF biosynthesis and/or secretion. These sequences do not directly code for the GAF molecule, which is a low molecular weight metabolite whose structure is described herein. The gene products encoded by these sequences are proteins involved in GAF-production. A prediction from that earlier work is that at least some of these sequences are specific to GAF-producing strains of bacteria, and a likely candidate for such a sequence appeared to be at least part of the 5,000-bp sequence associated with the GAF2 mutation described in the referenced patent application (the sequence corresponding to SEQ ID NO: 7 in U.S. application Ser. No. 10/537,017). Of particular interest in that 5 KB sequence was a 600-bp open-reading frame that was identified as coding for a putative formyl-transferase, and is set forth herein as SEQ ID NO: 2 (The amino acid sequence corresponding to this open reading frame was labeled SEQ ID NO: 8 in U.S. patent application Ser. No. 10/537,017). The product of this gene appeared likely to be an enzyme directly involved in some aspect of the GAF biosynthetic pathway, and as can be seen in Table 10, the sequence of this gene is rather distinctive relative to analogous genes in other organisms. The only gene in GenBank with relatively high homology to the WH6 sequence corresponded to a small formyltransferase (89 amino acids) from Marinobacter. The sequence of this gene exhibited a relatively high homology to a region representing approximately one-third of the much larger WII6 gene, which codes for a protein consisting of 200 amino acids. None of the gene sequences previously recorded for Pseudomonas species exhibited any close homology to the WH6 sequence.

TABLE 10
Amino Acid Sequences Homologous to the Putative Formyl-Transferase
from Pseudomonas fluorescens Isolate WH6.

| | % | | |
|---|---|---|---|
| | Amino Acid | | GenBank |
| | Identities/ | | Accession |
| ORGANISM | Positives | Ranking* | # |
| Marinobacter sp. ELB17 | 91/94 | 1 | ZP_01735306 |
| Rhodococcus fascians | 37/53 | 2 | CAC43337 |
| Prochlorococcus marinus | 36/58 | 3 | ZP_01005028 |
| str. MIT 9211 | | | |
| Photorhabdus temperata | 37/54 | 4 | AAR17609 |
| Listonella anguillarum | 38/58 | 5 | YP_001144382 |
| serovar O2 | | | |
| Pseudomonas syringae | 27/49 | 109 | YP_274991 |
| pv. phaseolicola 1448A | | | |
| Pseudomonas stutzeri | 28/48 | 174 | YP_001173319 |
| A1501 | | | |
| Pseudomonas syringae | 26/48 | 186 | YP_235768 |
| pv. syringae B728a | | | |
| Pseudomonas mendocina | 31/52 | 214 | YP_001188484 |
| ymp | | | |
| Pseudomonas fluorescens | 29/49 | 240 | YP_348574 |
| PfO-1 | | | |
| Pseudomonas fluorescens | 29/48 | 259 | YP_260151 |
| Pf-5 | | | |
| Pseudomonas putida | 30/51 | 531 | YP_001269362 |
| F1 | | | |
| Pseudomonas putida W619 | 27/49 | 536 | ZP_01641541 |
| Pseudomonas putida GB-1 | 33/54 | 570 | ZP_01714946 |

*According to degree of homology to the WH6 gene. It should be noted that, as explained in the text, the Marinobacter gene was much smaller than the WH6 gene.

A translated nucleotide BLASTX query (Altschul et al., 1997) was performed on the nucleotide sequence set forth as SEQ ID NO: 2. The five organisms in the database with genes exhibiting the most significant degree of amino acid homology to the WH6 sequence are listed in order of rank. The percent identical amino acids and conserved amino acid substitutions (Positives) in each alignment are indicated. For comparison, the corresponding data for various Pseudomonas strains in GenBank, including P. fluorescens strains PfO-1 and Pf-5, are also listed. The WH6 amino acid sequence referenced here corresponds to SEQ ID NO: 8 of U.S. patent application Ser. No. 10/537,017.

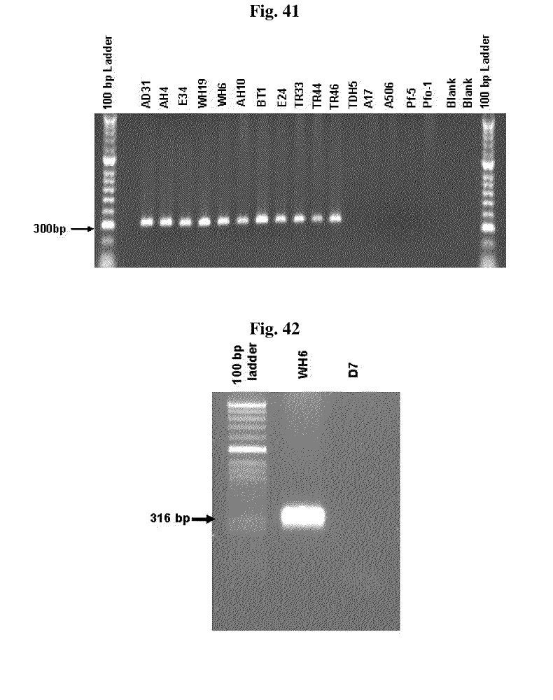

PCR primers corresponding to distinctive regions of the 600 by open reading frame constituting the WH6 formyl-transferase gene (i.e. the 600 by sequence corresponding to amino acid SEQ ID NO: 8 in U.S. application Ser. No. 10/537,017) were designed to probe other Pseudomonas isolates for a similar gene construct. The sequences of the WH6 formyl-transferase gene and the regions from which the PCR primers were designed are shown as SEQ ID NO: 2 and SEQ ID NOs 3 and 4, respectively. The results of the subsequent PCR analysis of DNA prepared from the various Pseudomonas isolates and strains used in this study, with the exception of P. fluorescens Strain D7, are shown in FIG. 41. All of the isolates shown to produce GAF (Table 9) were also found to contain a DNA sequence of the predicted size and primer specificity corresponding to the WH6 formyl-transferase sequence. On the other hand, this sequence could not be detected in any of the Pseudomonas isolates and strains that yielded culture filtrates that were inactive in the Poa bioassay for GAF activity (TDH5, A17, A506, Pf-5, and PfO-1).

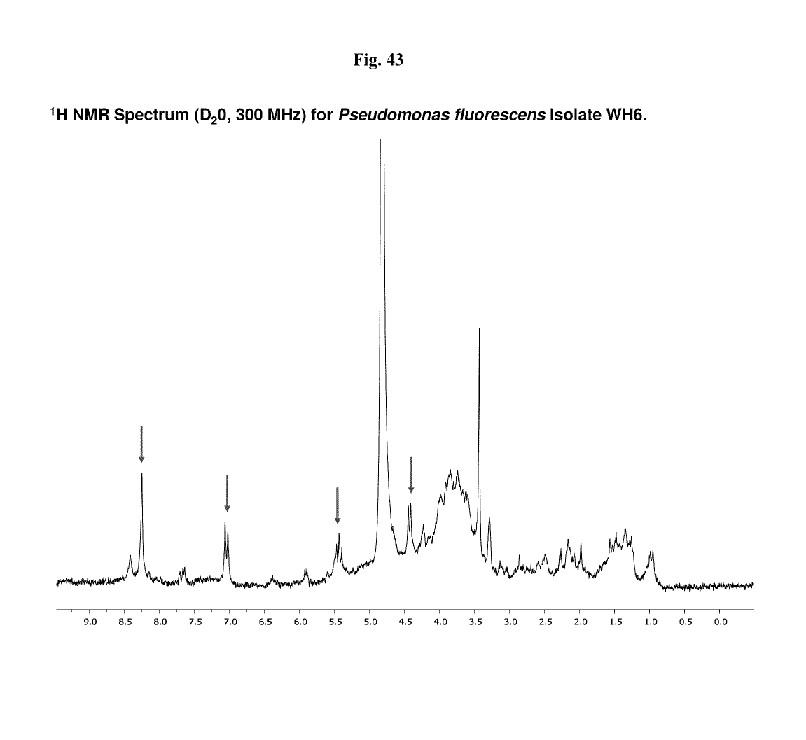

The PCR data for P. fluorescens D7 are shown in FIG. 42. As in the case of other Pseudomonas strains that do not produce GAF, a sequence corresponding to the WH6 formyl-transferase sequence could not be detected in P. fluorescens D7. Thus, one of the traits that appears to distinguish Pseudomonas strains that produce GAF from Pseudomonas strains that lack this capability is the presence of the DNA sequence shown SEQ ID NO: 2, and as evidenced by PCR detection of an appropriately sized DNA fragment after amplification with the PCR-primers set forth as SEQ ID NOs: 3 and 4.

### SUMMARY

Evidence contained herein establishes that Pseudomonas bacteria that produce GAF share certain features that distinguish them from other Pseudomonas strains and isolates, including some pseudomonads that also have deleterious effects on grassy weeds. As summarized in Table 11, these features include:
1. The ability of culture filtrates from GAF-producing Pseudomonas organisms to produce distinctive biological effects on seeds of Poa annua tested under the conditions specified here.

We have termed these effects Germination-Arrest, and they are characterized by an irreversible halt to germination at a developmental stage immediately following emergence of the plumule and coleorhiza from the seed coat (Germination Score of 1.0 as described in Example 1) and before these structures have exceeded the length of the seed. Although other strains of Pseudomonas (such as P. fluorescens D7, cited by the patent examiner) may retard or stunt the development of Poa annua seedlings or to some extent inhibit seed germination, they do not produce the precise syndrome of biological effects associated with culture filtrates from GAF-producing organisms.
2. The production of a distinctive low molecular weight compound that is responsible for the biological effects described above.

We have termed this compound a Germination Arrest Factor (GAF), and we have identified this compound as 2-amino-4-formylaminooxy-but-3-enoic acid (also called 2-amino-4-formylaminooxyvinylglycine). The presence of this compound in culture filtrates of Pseudomonas can be assessed by extraction and chromatography procedures described in Example 1. As described above, GAF is a low molecular weight, ninhydrin-reactive compound that possesses an anionic group, and is insoluble in organic solvents that are immiscible in water. Moreover, GAF has particular chromatographic properties that allow it to be detected in the culture filtrates of GAF-producing organisms as a distinctive ninhydrin-staining band located at a particular position on thin-layer chromatography plates when the extraction and chromatography protocols specified here are followed. This band is characteristic of Pseudomonas bacteria that exhibit GAF activity, and it cannot be detected in Pseudomonas isolates and strains that lack GAF activity, including P. fluorescens D7.
3. The presence of a DNA sequence that codes for an open reading frame that is distinctive for a particular putative formyl-transferase.

The DNA sequence shown as SEQ ID NO: 2appears to code for a distinctive formyl-transferase that, at least among pseudomonads, is characteristic of GAF-producing bacterial isolates. By this criterion, it is absent in all Pseudomonas isolates and strains that lack the ability to produce GAF. Sequences from this gene can be used to detect all GAF-producing Pseudomonas isolates by PCR-analysis using, for instance, the primers set forth as SEQ ID NOs: 3 and 4.

Thus, the evidence summarized here indicates that the three characteristics listed above are shared by all GAF-producing pseudomonads and can be used to distinguish GAF-producing Pseudomonas isolates and strains from other Pseudomonas bacteria.

Evidence that GAF-producing Pseudomonas isolates and strains may be distinguished from other pseuodomonads is summarized in Table 11.

TABLE 11
Summary of the Distribution of GAF-Related Traits among
Various Pseudomonas Isolates and Strains.

| | | NINHYDRIN- | |
|---|---|---|---|
| | | REACTIVE | |
| | | TLC-BAND | |
| ISOLATE | | AT Rf | GAF-SPECIFIC |
| OR | GAF | OF WH6 | FORMYLTRANSFERASE |
| STRAIN | ACTIVITY | GAF-BAND | GENE SEQUENCE |
| GROUP I | | | |
| AD31 | YES | PRESENT | PRESENT |
| AH4 | YES | PRESENT | PRESENT |
| E34 | YES | PRESENT | PRESENT |
| WH6 | YES | PRESENT | PRESENT |
| WH19 | YES | PRESENT | PRESENT |
| GROUP II | | | |
| AH10 | YES | PRESENT | PRESENT |
| BT1 | WEAK | WEAK | PRESENT |
| E24 | YES | PRESENT | PRESENT |
| TDH5 | NEGLIGIBLE | ABSENT | ABSENT |
| TR33 | YES | PRESENT | PRESENT |
| TR44 | YES | PRESENT | PRESENT |
| TR46 | YES | PRESENT | PRESENT |
| STRAIN | | | |
| Pf5 | NO | ABSENT | ABSENT |
| PfO-1 | NO | ABSENT | ABSENT |
| A506 | NO | ABSENT | ABSENT |
| A17 | NO | ABSENT | ABSENT |
| D7 | NO | ABSENT | ABSENT |

### Example 6

### Identification of GAF Activity in Additional Bacterial Strains

This example presents the characterization of additional bacterial strains having GAF activity using the Poa bioassay and analytical TLC chromatography.

A number of additional isolates of Pseudomonas fluorescens and other bacterial species have been tested for GAF activity. The culture filtrates from these isolates and strains have been tested for GAF activity and for the presence of FVG by analytical TLC chromatography as well as by ¹H-NMR (see Example 7). The specific sources of these bacterial cell lines and the criteria used for their taxonomic identification are shown in Tables 12 and 13. All of the isolates listed here were obtained from soils of the Willamette Valley and were selected on the basis of initial tests that indicated that the live bacteria had some herbicidal activity against grassy weeds.

TABLE 12
Origin and Taxonomic Identification of Additional Characterized
Pseudomonas fluorescens Isolates. Isolates with GAF activity
that have been shown to produce FVG are marked with an asterisk.

| | | RHIZOSPHERE | TAXONOMIC IDENTIFICATION | |
|---|---|---|---|---|
| | | SOURCE | FAME | rDNA |
| ISOLATE | SITE OF ISOLATION | OF ISOLATE | ANALYSIS | SEQUENCING |
| A3422A* | Disturbed Site, Alsea Valley | Unknown | Pseudomonas fluorescens | Pseudomonas |
| | Benton County, OR | | Biotype G | fluorescens |
| AH7 | Alsea Valley | Poa Species | Pseudomonas fluorescens | Pseudomonas |
| | Benton county, OR | | or P. putida Biotype B | fluorescens |
| ALW38* | Lawn, Alsea Valley | Poa Species | Pseudomonas fluorescens | Pseudomonas |
| | Benton County, OR | | Biotype G | fluorescens |
| G2Y* | Grower's Field | Lolium perenne | Pseudomonas putida | Pseudomonas |
| | Linn County, OR | | Biotype B | fluorescens |
| GTR12* | Edge of Compost Pile, Farm | Grassy Weeds | Pseudomonas fluorescens | Pseudomonas |
| | Philomath, Benton County, OR | | Biotype B | fluorescens |
| GTR24* | Edge of Compost Pile, Farm | Grassy Weeds | Pseudomonas fluorescens | Pseudomonas |
| | Philomath, Benton County, OR | | Biotype B | fluorescens |
| GTR40* | Philomath, Benton County, OR | Grassy Weeds | Pseudomonas fluorescens | Pseudomonas |
| | Edge of Compost Pile, Farm | | Biotype B | fluorescens |
| | Philomath, Benton County, OR | | | |
| HB14* | Lawn, Alsea Valley | Poa Species | Pseudomonas putida | Pseudomonas |
| | Benton County, OR | | Biotype B | fluorescens |
| HB26* | Lawn, Alsea Valley | Poa Species | Pseudomonas putida | Pseudomonas |
| | Benton County, OR | | Biotype B | fluorescens |
| HB32* | Lawn, Alsea Valley | Poa Species | Pseudomonas putida | Pseudomonas |
| | Benton County, OR | | Biotype B | fluorescens |
| ST22* | Hyslop Research Farm, OSU | Hordeum vulgare | Pseudomonas fluorescens | Pseudomonas |
| | Benton County, OR | | Biotype G | fluorescens |
| W36* | Hyslop Research Farm, OSU | Triticum | Pseudomonas fluorescens | Pseudomonas |
| | Benton County, OR | (with Poa species) | Biotype G | fluorescens |

TABLE 13
Origin and Taxonomic Identification of Isolates of Additional Characterized
Bacterial Species (other than P. fluorescens). Isolates with GAF activity
that have been shown to produce FVG are marked with an asterisk.

| | | RHIZOSPHERE | TAXONOMIC IDENTIFICATION | |
|---|---|---|---|---|
| | | SOURCE | FAME | rDNA |
| ISOLATE | SITE OF ISOLATION | OF ISOLATE | ANALYSIS | SEQUENCING |
| A3203* | Willamette Valley, OR | Unkown | Enterobacter sp. | Enterobacter kobei |
| A342* | Willamette Valley, OR | Unknown | Pseudomonas fluorescens | Pseudomonas |
| | | | Biotype G | mucidolens |
| | | | | or P. synxantha |
| AH18 | Lawn, Alsea Valley | Poa Species | Pseudomonas fluorescens | Pseudomonas |
| | Benton county, OR | | Biotype B | mucidolens |
| | | | | or P. synxantha |
| BW1 | Hyslop Research Farm, OSU | Triticum | Pseudomonas fluorescens | Pseudomonas poae |
| | Benton County, OR | (with Poa species) | Biotype B | or P. trivialis |
| GTR20-18-2 | Edge of Compost Pile, Farm | Grassy Weeds | Enterobacter intermedius | Enterobacter |
| | Philomath, Benton County, OR | | | amnigenus |
| GTR28 | Edge of Compost Pile, Farm | Grassy Weeds | Pseudomonas putida | Pseudomonas veronii |
| | Philomath, Benton County, OR | | Biotype A | |
| L2-1-1 | Organic Vegetable Farm | Poa species | Enterobacter | Enterobacter |
| | Philomath, Benton County, OR | | intermedius | asburiae |
| TDH40* | Organic Vegetable Farm | Poa species | Pseudomonas fluorescens | Pseudomonas |
| | Philomath, Benton County, OR | | Biotype B | mucidolens |
| | | | | or P. synxantha |

Culture filtrates from eleven of twelve P. fluorescens isolates and strains tested in our standard Poa bioassay were found to possess GAF activity (Table 14), and three of eight bacterial isolates identified as belonging to other species also yielded culture filtrates that exhibited GAF activity in this bioassay (Table 15).

TABLE 14
Germination-Arrest Activity of Culture Filtrates from Additional
Pseudomonas fluorescens Isolates and Strains Tested in the Standard
Poa Bioassay. Isolates with GAF activity that have been shown to
produce FVG are marked with an asterisk.

| | MEAN GERMINATION SCORE IN THE | | |
|---|---|---|---|
| | POA BIOASSAY AT THE | | |
| | INDICATED CULTURE FILTRATE DILUTION | | |
| | (±Standard Error of the Mean) | | |
| | 0.1X | 0.3X | 1.0X (Full-Strength) |
| CONTROLS | | | |
| PMS Medium | 4.0 ± 0.00 | 4.0 ± 0.00 | 4.0 ± 0.00 |
| WH6* | 1.0 ± 0.00 | 1.0 ± 0.00 | 1.0 ± 0.00 |
| A3422A* | 1.0 ± 0.00 | 1.0 ± 0.00 | 1.0 ± 0.00 |
| AH7 | 4.0 ± 0.00 | 4.0 ± 0.00 | 3.0 ± 0.17 |
| ALW38* | 1.4 ± 0.06 | 1.0 ± 0.00 | 1.0 ± 0.00 |
| G2Y* | 1.9 ± 0.21 | 1.0 ± 0.00 | 1.0 ± 0.00 |
| GTR12* | 3.3 ± 0.26 | 1.0 ± 0.00 | 1.0 ± 0.00 |
| GTR24* | 1.2 ± 0.13 | 1.0 ± 0.00 | 1.0 ± 0.00 |
| GTR40* | 1.1 ± 0.08 | 1.0 ± 0.00 | 1.0 ± 0.00 |
| HB14* | 1.0 ± 0.00 | 1.0 ± 0.00 | 1.0 ± 0.00 |
| HB26* | 1.4 ± 0.07 | 1.0 ± 0.00 | 1.0 ± 0.00 |
| HB32* | 1.2 ± 0.13 | 1.0 ± 0.00 | 1.0 ± 0.00 |
| ST22* | 1.8 ± 0.20 | 1.0 ± 0.00 | 1.0 ± 0.00 |
| W36* | 1.0 ± 0.00 | 1.0 ± 0.00 | 1.0 ± 0.00 |

TABLE 15
Germination-Arrest Activity of Culture Filtrates from Additional
Bacterial Species (Other than P. fluorescens) Tested in the
Standard Poa Bioassay. Isolates with GAF activity that have
been shown to produce FVG are marked with an asterisk.

| | | MEAN GERMINATION SCORE | | |
|---|---|---|---|---|
| SOURCE OF | | IN THE POA BIOASSAY | | |
| CULTURE | | AT THE INDICATED CULTURE | | |
| FILTRATE | | FILTRATE DILUTION | | |
| | SPECIES | (±Standard Error of the Mean) | | |
| | (from rDNA | | | 1.0X |
| ISOLATE | Sequencing) | 0.1X | 0.3X | (Full-Strength) |
| Controls | | | | |
| PMS | — | 4.0 ± 0.00 | 4.0 ± 0.00 | 4.0 ± 0.00 |
| Medium | | | | |
| WH6* | P. fluorescens | 1.0 ± 0.00 | 1.0 ± 0.00 | 1.0 ± 0.00 |
| A3203* | Enterobacter kobei | 1.2 ± 0.00 | 1.0 ± 0.00 | 1.0 ± 0.00 |
| A342* | P. mucidolens | 1.5 ± 0.00 | 1.0 ± 0.00 | 1.0 ± 0.00 |
| | or P. synxantha | | | |
| AH18 | P. mucidolens | 4.0 ± 0.00 | 4.0 ± 0.00 | 2.5 ± 0.17 |
| | or P. synxantha | | | |
| BW1 | P. poae | 4.0 ± 0.00 | 4.0 ± 0.00 | 4.0 ± 0.00 |
| | or P. trivialis | | | |
| GTR20- | Enterobacter | 4.0 ± 0.00 | 4.0 ± 0.00 | 4.0 ± 0.00 |
| 18-2 | amnigenus | | | |
| GTR28 | P. veronii | 4.0 ± 0.00 | 4.0 ± 0.00 | 4.0 ± 0.00 |
| L2-1-1 | Enterobacter | 4.0 ± 0.00 | 4.0 ± 0.00 | 4.0 ± 0.00 |
| | asburiae | | | |
| TDH40* | P. mucidolens | 1.5 ± 0.00 | 1.0 ± 0.00 | 1.0 ± 0.00 |
| | or P. synxantha | | | |

All of the culture filtrates screened for GAF activity were subsequently analyzed by thin-layer chromatography (TLC) for the characteristic ninhydrin reactive FVG band. TLC analysis of culture filtrates prepared from the isolates and strains listed in Tables 12 and 13 demonstrated that all of the culture filtrates that exhibited GAF activity contained the expected ninhydrin-reactive band characteristic of FVG. This band was absent from the isolates and strains that were inactive in the Poa bioassay (e.g. isolates AH7, AH18, BW1, GTR20-18-2, and L2-1-1).

### Example 7

### Detection of Characteristic ¹H NMR Spectrum in Bacteria with GAF Activity

This example shows that the characteristic ¹H NMR pattern of purified GAF (FVG) described in Example 2 is dectable in the culture filtrate of bacteria with GAF activity.

### Methods

NMR Spectroscopy: Samples of the culture filtrate extracts prepared for TLC analysis were taken for ¹H NMR data acquisition. For this purpose, 1.4 mL aliquots of the 20-fold concentrated 76% ethanol solutions, prepared as described above, were concentrated in vacuo at 37° C. and placed under high vacuum for up to 24 hr to remove all traces of protonated solvent. The resulting dry samples were suspended in 20 microliters of deuterated water (D₂O). These extremely concentrated solutions were filtered through a 2 micron Upchurch Scientific Mini Microfilter before 5-10 microliters of the resulting filtrate were manually injected through a second inline microfilter into a Protasis microflow capillary NMR probe (2.5 microliter active coil volume; 5 microliter flow cell) installed in a Bruker Avance DRX300 NMR spectrometer. ¹H NMR spectra were acquired in 256 scans at 298K on each sample and internally referenced to the residual HOD solvent peak calibrated to δ_{H} 4.80 ppm.

### Results

Nuclear magnetic resonance (NMR) spectroscopy of crude extracts prepared from culture filtrates of these bacterial isolates and strains confirmed the presence of FVG in all of the culture filtrates that exhibited GAF activity activity. As described in Example 2 and as illustrated in FIG. 13, the ¹H NMR spectrum of purified GAF (FVG) in D₂O exhibits characteristic signals at δ_{H} 7.70, 4.26, 5.22, and 6.96 ppm corresponding respectively to the formyl group proton (hydrogen) of FVG, and the protons associated with the alpha (CH-2, beta (CH-3), and gamma (CH-4) positions in FVG. The latter three signals are characteristic of all oxyvinylglycines, while the former is specific for FVG itself. Four corresponding FVG proton signals are also clearly visible in the NMR spectrum of a crude 90% ethanol extract of dried culture filtrate from WH6 (FIG. 43). Note that in the spectra of the 90% ethanol extract of WH6 and other FVG-containing isolates, the four proton chemical shifts are deshielded (δ_{H} 8.19-8.25, 4.34-4.42, 5.37-5.43, and 6.98-7.03 ppm) relative to that in the spectrum for the purified FVG (δ_{H} 7.70, 4.26, 5.22, and 6.96 ppm). This is due to the known electronic effects (induced dipoles) of counter ions supplied by the different salt compositions in the crude and purified materials.

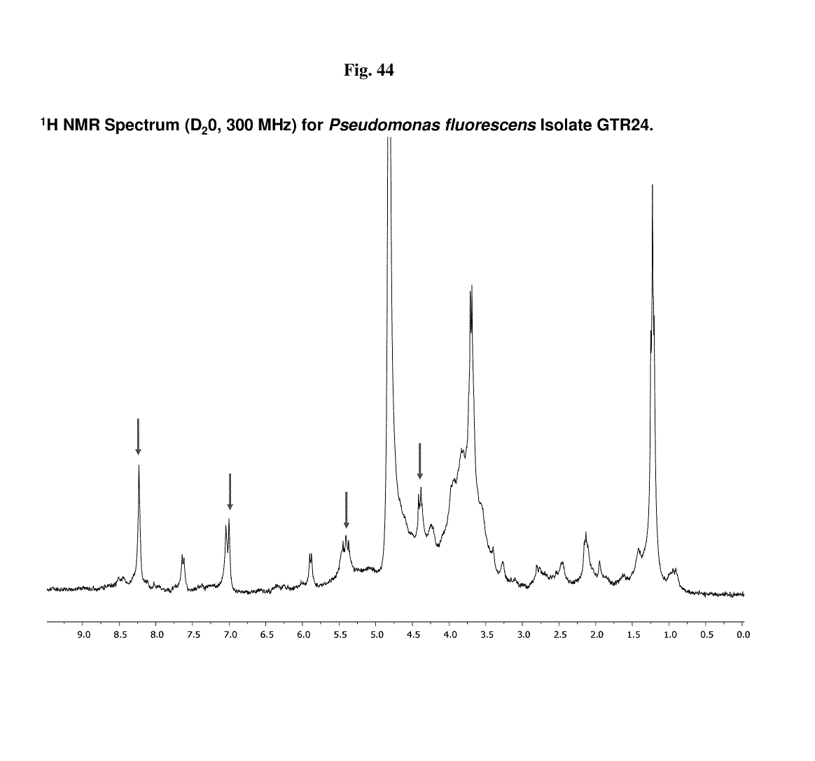

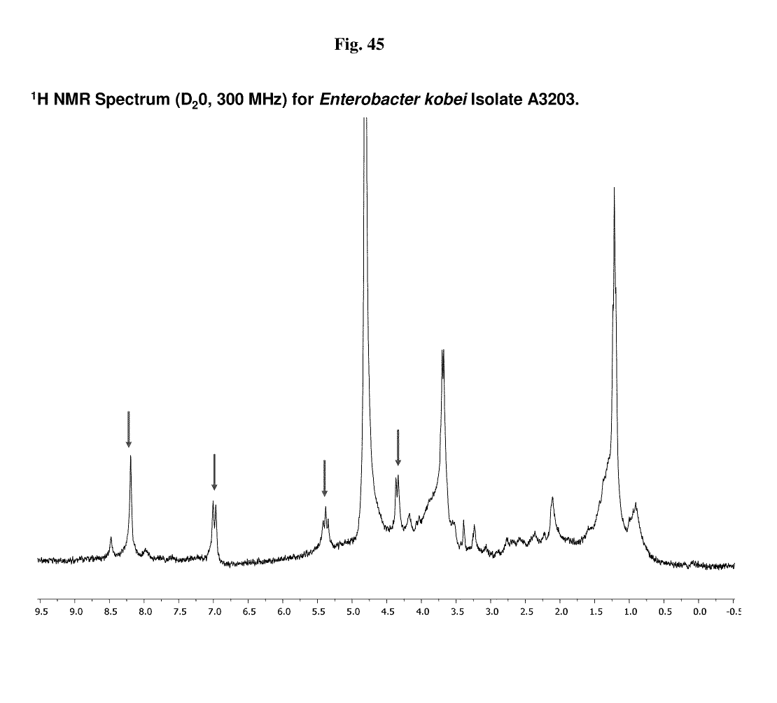

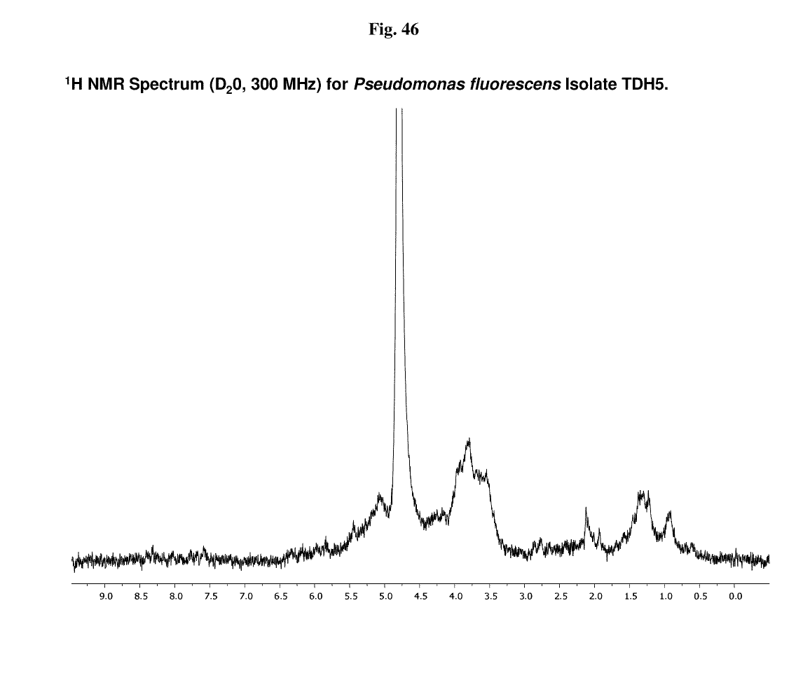

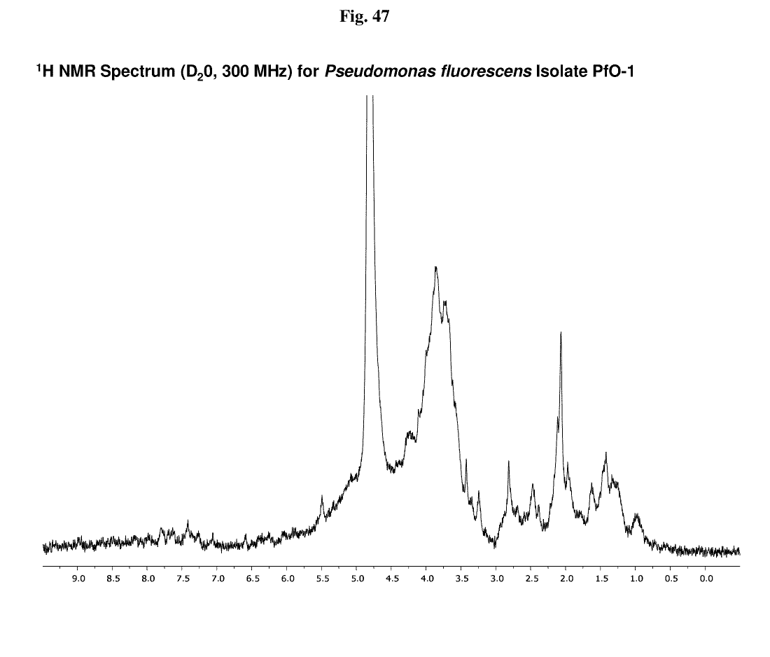

FIGS. 44 and 45 illustrate the NMR spectra obtained on crude extracts of the culture filtrates for two other FVG-producing isolates (Pseudomonas fluorescens Isolate GTR24 and Enterobacter kobei Isolate A3203). The arrows indicate the four distinctive NMR signals for FVG. In comparison, FIGS. 46 and 47 show the NMR spectra for two bacterial lines (P. fluorescens Isolate TDH5 and P. fluorescens Strain PfO-1) that lack the biological activity and TLC-banding pattern expected for FVG-producing bacteria. The four ¹H NMR signals characteristic of FVG are absent from these spectra. The analogous data were obtained for all of the bacterial isolates and strains described in Examples 5 and 6. In all cases, the ¹H NMR signals characteristic of FVG could be detected in extracts of culture filtrates from bacterial lines that exhibited GAF activity, while those signals were absent from the corresponding extracts prepared from culture filtrates of bacterial isolates and strains that lacked the indicated biological activities.

It will be apparent that the precise details of the methods or compositions described herein may be varied or modified without departing from the spirit of the described disclosure. We claim all such modifications and variations that fall within the scope and spirit of the claims below.

We claim:

1. An isolated Germination-Arrest Factor produced by a strain of bacteria or by chemical synthesis, wherein the Germination-Arrest Factor comprises a vinylglycine molecule that inhibits or arrests the germination of grassy weed seeds.

2. The Germination-Arrest Factor of claim 1, wherein the genome of the bacteria that produces the isolated Germination Arrest Factor comprises a formyl-transferase gene having a sequence at least 80% identical to SEQ ID NO: 2.

3. The Germination-Arrest Factor of claim 1, wherein the bacterium is selected from the group consisting of Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, and Enterobacter kobei A3203.

4. The Germination-Arrest Factor of claim 1, wherein the vinylglycine molecule is 2-amino-4-formylaminooxy-but-3-enoic acid (also named 4-formylaminooxyvinylglycine).

5. A structurally related compound, analog, or derivative of the Germination-Arrest Factor of claim 4, wherein the vinylglycine molecule has the structure of one of Formulae IA, IB or IC, and is effective to inhibit or arrest weed seed germination: 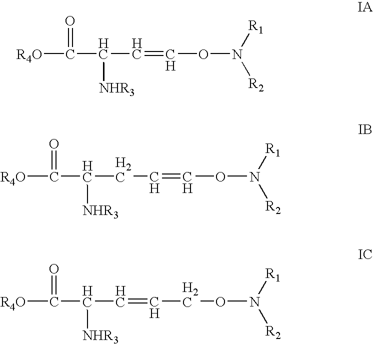
wherein R₁ and R₂ independently are selected from H, optionally substituted lower aliphatic, optionally substituted amino, alkoxy, hydroxy, —COOH, sulfonic acid hydroxy alkyl, alkyl amino;
R₃ independently is selected from H, acetyl, propanyl, and optionally substituted lower aliphatic;
R₄ independently is selected from H and optionally substituted lower aliphatic.

6. A structurally related compound, analog, or derivative of the Germination-Arrest Factor of claim 4, wherein the vinylglycine molecule has the structure of one of Formulae IIA, IIB or IIC, and is effective to inhibit or arrest weed seed germination: 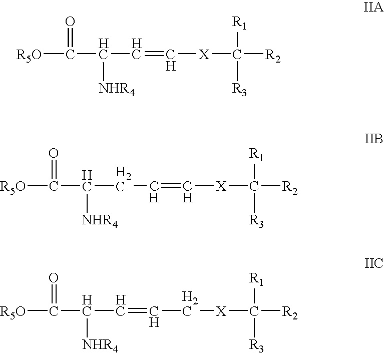
wherein X is selected from O, S, and optionally substituted amino;
R₁, R₂, and R₃ independently area selected from H, optionally substituted lower aliphatic, optionally substituted amino, alkoxy, hydroxy, —COOH, —NHCOOH, sulfonic acid, hydroxy alkyl, alkyl amino;
R₄ independently is selected from H, acetyl, propanyl and optionally substituted lower aliphatic;
R₅ independently is selected from H and optionally substituted lower aliphatic.

7. A structurally related compound, analog, or derivative of the Germination-Arrest Factor of claim 4, wherein the resulting molecule has the structure of Formula III and is effective to inhibit or arrest weed seed germination: 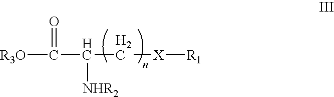
wherein X is selected from O, S and optionally substituted amino; n is one, two, three, four, or five;
R₁ independently is selected from optionally substituted lower aliphatic, optionally substituted amino, alkoxy, hydroxy, —NHCOOH, sulfonic acid, hydroxy alkyl, alkyl amino;
R₂ independently is selected from H, acetyl, propanyl, and optionally substituted lower aliphatic; and
R₃ independently is selected from H and optionally substituted lower aliphatic.

8. A structurally related compound, analog, or derivative of the Germination-Arrest Factor of claim 4, wherein the vinylglycine molecule has the structure of Formula IV and is effective to inhibit or arrest weed seed germination: 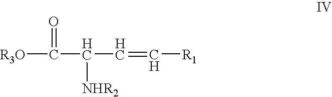
wherein
R₁═NHOH, R₂═H, CH₃, COCH₃, R₃═H, CH₃, CH₃CH₂
R₁═NHOCH₃, R₂═H, CH₃, COCH₃, R₃═H, CH₃, CH₃CH₂
R₁=OCH₂CH₂NH₂, R₂═H, CH₃, COCH₃, R₃═H, CH₃, CH₃CH₂
R₁═OCH₃, R₂═H, CH₃, COCH₃, R₃═H, CH₃, CH₃CH₂
R₁═ONH₂, R₂═H, CH₃, COCH₃, R₃═H, CH₃, CH₃CH₂
R₁═CH₂PO₃²⁻, R₂═H, CH₃, COCH₃, R₃═H, CH₃, CH₃CH₂
R₁═CH₂CH₂CO₂H, R₂═H, CH₃, COCH₃, R₃═H, CH₃, CH₃CH₂
R₁═CHCH₂, R₂═H, CH₃, COCH₃, R₃═H, CH₃, CH₃CH₂
R₁═CH₂NHCONH₂, R₂═H, CH₃, COCH₃, R₃═H, CH₃, CH₃CH₂ 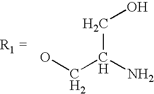
R₂═H, CH₃, COCH₃, R₃═H, CH₃, CH₃CH₂ 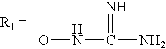
R₂═H, CH₃, COCH₃, R₃═H, CH₃, CH₃CH₂ 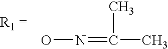
R₂═H, CH₃, COCH₃, R₃═H, CH₃, CH₃CH₂
or wherein the compositions include alkyloxy and aryloxy vinylglycines, including those wherein
R₁═OCH₂CH(CH₃)CH₃, SCH₃, R₂═H, CH₃, COCH₃, R₃═H, CH₃, CH₃CH₂ 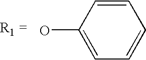
R₂═H, CH₃, COCH₃, R₃═H, CH₃, CH₃CH₂ 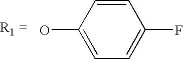
R₂═H, CH₃, COCH₃, R₃═H, CH₃, CH₃CH₂ 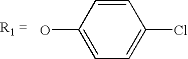
R₂═H, CH₃, COCH₃, R₃═H, CH₃, CH₃CH₂

9. A structurally related compound, analog, or derivative of the Germination-Arrest Factor of claim 4, wherein the vinylglycine molecule is effective to inhibit weed seed germination and has the structure of one of formulae 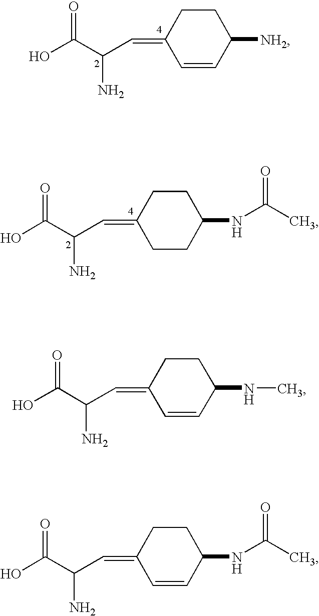
or any of the above formulae wherein the carboxyl at position 1 is optionally substituted with a sulfonic acid group.

10. A structurally related compound, analog, or derivative of the Germination-Arrest Factor of claim 4, wherein the vinylglycine molecule is effective to inhibit weed seed germination and has the structure of Formula V: 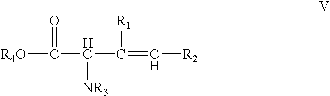
wherein the substituents R₁ and R₂ independently are selected from optionally substituted lower aliphatic and halo, R₃ is selected from H, lower alkyl, acetyl and propanyl, and R₄ is selected from H and lower alkyl.

11. The Germination-Arrest Factor of claim 1, wherein the weed is Poa annua, Poa trivialis or Bromus tectorum.

12. The Germination-Arrest Factor of claim 1, wherein the weed is crabgrass, goosegrass, dallisgrass, bahiagrass, annual bluegrass, downy brome, jointed goatgrass, roughstalk bluegrass, rattail fescue, perennial ryegrass, or tall fescue.

13. A method for inhibiting or arresting weed germination in a growth medium in which it would be desirable to inhibit or arrest weed germination, the method comprising applying the isolated Germination-Arrest Factor of claim 1 to the growth medium in an amount sufficient to inhibit or arrest grassy weed germination.

14. The method of claim 13, wherein the isolated Germination Arrest Factor is produced by a strain of bacteria, the genome of which comprises a formyl-transferase gene having a sequence at least 80% identical to SEQ ID NO: 2.

15. The method of claim 13, wherein the isolated Germination Arrest Factor is produced by a strain of bacteria selected from the group consisting of Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDH40, and Enterobacter kobei A3203.

16. The method of claim 13, wherein the isolated Germination-Arrest Factor is applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide.

17. The method of claim 13, wherein the isolated Germination-Arrest Factor is applied in a formulation that is a solution, a soluble powder, an emulsifiable concentrate, a wettable powder, a liquid flowable, a dry flowable, a water-dispersible granule, a granule, or a pellet.

18. A method of inhibiting or arresting weed germination among seeds that are insensitive to the Germination-Arrest Factor, the method comprising applying the isolated Germination-Arrest Factor of claim 1 to the GAF-insensitive seeds in an amount sufficient to inhibit or arrest germination of any grassy weed seeds that are mixed in with the GAF-insensitive seeds.

19. The method of claim 18, wherein the isolated Germination-Arrest Factor is applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide.

20. The method of claim 18, wherein the isolated Germination-Arrest Factor or is applied in a formulation that is a solution, a soluble powder, an emulsifiable concentrate, a wettable powder, a liquid flowable, a dry flowable, a water-dispersible granule, a granule, or a pellet.

21. A composition for inhibiting or arresting the germination of weeds, comprising:
the isolated Germination-Arrest Factor of claim 1; and
a timed- or temperature-release coating over at least a portion of the isolated Germination-Arrest Factor.

22. The composition of claim 21, further comprising a water-resistant coating over the timed-or temperature-release coating.

23. A method of inhibiting or arresting weed germination in an area in which inhibiting or arresting weed germination is desirable, comprising:
broadcasting an herbicidally effective amount of the isolated Germination-Arrest Factor of claim 1 at least once a year across the area, thereby inhibiting or arresting weed germination in the area.

24. The method of claim 23, wherein the isolated Germination-Arrest Factor of claim 1 is applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide.

25. The method of claim 23, wherein the isolated Germination-Arrest Factor of claim 1 is applied in a formulation that is a solution, a soluble powder, an emulsifiable concentrate, a wettable powder, a liquid flowable, a dry flowable, a water-dispersible granule, a granule, or a pellet.

26. The method of claim 25, wherein the isolated Germination-Arrest Factor of claim 1 is formulated as a granule.

27. The method of claim 26, wherein the granule is at least partially coated with a timed-or temperature-release coating.

28. The method of claim 27, wherein the timed-or temperature-release coating is coated with a water-resistant coating.

29. The method of claim 23, wherein the method is a method of inhibiting grassy weeds among dicot species or among established monocot seedlings.

30. A method of producing the isolated Germination-Arrest Factor of claim 1, comprising:
culturing Pseudomonas fluorescens isolates WH6, AD31, AH4, E34, WH19, AH10, BT1, E24, TR33, TR44, TR46, A3422A, ALW38, G2Y, GTR12, GTR24, GTR40, HB14, HB26, HB32, ST22, W36, Pseudomonas mucidolens/synxantha isolates A342, TDII40, or Enterobacter kobei A3203 in a suitable culture medium;
collecting the culture medium; and
purifying the culture medium to produce the Germination-Arrest Factor.

31. A method of producing the isolated Germination-Arrest Factor of claim 1, comprising:
culturing a bacterium in a suitable culture medium, wherein the genome of the bacterium comprises a formyl-transferase gene having a sequence at least 80% identical to SEQ ID NO: 2;
collecting the culture medium; and
purifying the culture medium to produce the isolated Germination-Arrest Factor.

32. A kit for inhibiting or arresting weed germination or growth, comprising:
the isolated Germination-Arrest Factor of claim 1; and
a container, and optionally, instructions for using the kit.

33. The isolated Germination-Arrest Factor of claim 1 for use as a seed-cleaning adjuvant in seed-cleaning processes as a supplement or alternative to physical removal of target weed seeds.

34. The method of claim 13 wherein an inhibitor of pyridoxal phosphate-dependent enzyme reactions other than a vinylglycine is substituted for the Germination-Arrest Factor.

35. The method of claim 34, wherein the inhibitor is aminooxyacetic acid or formylaminooxyacetic acid.


SEQUENCE LISTING (4 sequences)

SEQ ID NO: 1 - DNA, 417 bp, Pseudomonas fluorescens
catgcaagtcgagcggtagagagaagcttgcttctcttgagagcggcggacgggtgagtaaagcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaaacctgatccagccatgccgcgtgtgtgaagaaggtcttcggattgtaaagcactttaagttgggaggaagggttgtagattaatactctgcaa

SEQ ID NO: 2 - DNA, 603 bp, Pseudomonas fluorescens
atgcagacaatcgagccatgcaaggacaaggtcctgctgtcggtgtgcaccatggactggtgcgatcacggcgtggaattcgccaagaccgtattctccaaccttgaggtgttttgctgggacccgggcgatccctatccttaccatctggataactgggaaggcgattggatcatctcctaccgtggcgacttcatttttccgccgagcatctacaagaatgcccgcaaaggcgcgatcaatctgcacccggcaccgcccaagtaccggggcctgggcagccagcattacgcgatctactacaacgatgaaacctacggctcgacctgccatcacctggccccttcggtggacagcgggcagatcatcaatgtggcgcgtttcaacgtcgcacccgccgagacggcttcatcgttgcgcctgcacgtaggtgcctattgcctgcagcagttcatccacctgctcaccgactacatcctgctgggacggccactgccggtctcgccggaaaattggggcgagcgcctgtacaagcaatcggaactcaagccctggatggagaagatccgcgcccaggagccggatcatcgttgcttcaaatag

SEQ ID NO: 3 - DNA, 18 bp, Artificial sequence
  Synthetic oligonucleotide
gacaatcgagccatgcaa

SEQ ID NO: 4 - DNA, 20 bp, Artificial sequence
  Synthetic oligonucleotide
gccgtaggtttcatcgttgt